SEQUENCE LISTING (30 sequences)

SEQ ID NO: 1 - protein, 443 aa, Artificial Sequence
  HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, Artificial Sequence
  HC(B)
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 215 aa, Artificial Sequence
  LC(B)
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 443 aa, Artificial Sequence
  HC(A)(K392DK409D)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 6 - protein, 450 aa, Artificial Sequence
  HC(B)(D399KE356K)
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 7 - protein, 440 aa, Artificial Sequence
  DIC014 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 8 - protein, 447 aa, Artificial Sequence
  DIC014 HC(B)
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSP

SEQ ID NO: 9 - protein, 214 aa, Artificial Sequence
  DIC002 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 10 - protein, 440 aa, Artificial Sequence
  DIC002 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 11 - protein, 440 aa, Artificial Sequence
  DIC007 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 12 - protein, 214 aa, Artificial Sequence
  DIC003 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFICPPSDEQLKSGTASVVCLLNNFYPREAKVQWKVDNALQSGNSQESVTEQDSKDSTYSLESTLTLSKADYEKHKVYACEVTHQGLSSPVTKSFNRGES

SEQ ID NO: 13 - protein, 440 aa, Artificial Sequence
  DIC003 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPCAPSSKSTSGGTAALGCLVKDYFPEPVTVSWNSGALTSGVHTFPAVLQSSGLYSLRSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 14 - protein, 214 aa, Artificial Sequence
  DIC004 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFICPPEDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 15 - protein, 440 aa, Artificial Sequence
  DIC004 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVRPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 16 - protein, 214 aa, Artificial Sequence
  DIC005 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVEICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 17 - protein, 440 aa, Artificial Sequence
  DIC005 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPCAPSSKSTSGGTARLGCLVKDYFPEPVTVSWNSGALTSGVHTFPAVLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 18 - protein, 214 aa, Artificial Sequence
  DIC006 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFICPPSKEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 19 - protein, 440 aa, Artificial Sequence
  DIC006 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPDSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 20 - protein, 214 aa, Artificial Sequence
  DIC009 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSCESVTEQDSKDSTYSLDSTLTLSKADYEKHKVYACEVTHQGLSSPVTKSFNRGES

SEQ ID NO: 21 - protein, 440 aa, Artificial Sequence
  DIC009 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPACLQSSGLYSLKSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 22 - protein, 214 aa, Artificial Sequence
  DIC010 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVDIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 23 - protein, 440 aa, Artificial Sequence
  DIC010 HC(A)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAKLGCLVKDYFPEPVTVSWNSGALTSGVHTFPACLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 24 - protein, 5 aa, Artificial Sequence
  repeated sequence for linker
TGGGG

SEQ ID NO: 25 - protein, 5 aa, Artificial Sequence
  repeated sequence for linker
GGGGS

SEQ ID NO: 26 - protein, 5 aa, Artificial Sequence
  repeated sequence for linker
SGGGG

SEQ ID NO: 27 - protein, 15 aa, Artificial Sequence
  linker
GGGGSGGGGSGGGGS

SEQ ID NO: 28 - protein, 18 aa, Artificial Sequence
  repeated sequence for linker
GAPGGGGGAAAAAGGGGG

SEQ ID NO: 29 - protein, 4 aa, Artificial Sequence
  repeated sequence for linker
GGGS

SEQ ID NO: 30 - protein, 214 aa, Artificial Sequence
  DIC007 LC(A)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

(19) United States
(12) Patent Application Publication
(10) Pub. No.: US 2024/0182576 A1
(43) Pub. Date: Jun. 6, 2024

(54) HETERODIMERIC ANTIBODIES AND ANTIGEN-BINDING FRAGMENT THEREOF

(71) Applicant: CHIMAGEN BIOSCIENCES, LTD, Shanghai (CN)

(72) Inventors: Zhenhao ZHOU, Shanghai (CN); Jie ZHANG, Shanghai (CN)

(21) Appl. No.: 18/556,665

(22) PCT Filed: Apr. 22, 2022
(86) PCT No.: PCT/CN2022/088386

§ 371 Date: Oct. 21, 2023

(30) Foreign Application Priority Data
Apr. 23, 2021 (WO) PCT/CN2021/089400

(51) Int. Cl. C07K 16/28; A61K 39/00; A61P 35/00

(52) U.S. Cl. CPC C07K 16/2818; A61P 35/00; C07K 16/2863; A61K 2039/505; C07K 2317/24; C07K 2317/31; C07K 2317/522; C07K 2317/55; C07K 2317/565

(57) ABSTRACT

Provided are novel polypeptide complexes that are useful for enhancing correct light chain pairing in a bispecific or multi-specific molecule. Further provided are nucleic acids comprising nucleotide sequences encoding the polypeptide complexes, vectors comprising the nucleic acids, host cells comprising the nucleic acids or the vectors, pharmaceutical compositions comprising the polypeptide complexes, as well as use of the polypeptide complexes for treating or preventing from diseases, conditions, or symptoms.

### FIELD OF THE INVENTION

The present disclosure relates generally to the field of protein engineering, specifically to the area of engineered antibodies, and in more particular to bispecific antibodies engineered to have high selectivity in the cognate pairing of immunoglobulin light and heavy chains.

### BACKGROUND

Antibodies, also known as immunoglobulins (Igs), are large proteins present in the plasma or other body fluid in a vertebrate that are produced by plasma cells (i.e. differentiated B cells) and utilized by the immune system to bind to and neutralize foreign agents (e.g. pathogenic bacteria, viruses, and parasites, etc.) invading the host. Primarily due to their capabilities to recognize and bind to various target molecules (i.e. antigens) in a highly specific manner, antibodies have long been used as a crucial research tool for scientific studies and an important disease screening and diagnostic tool, and have recently emerged as a promising therapeutic agent to manage or treat a variety of human diseases, including certain autoimmune/inflammatory disorders such as psoriasis, rheumatoid arthritis, and multiple sclerosis, etc., and most notably cancers (e.g. breast cancer, colorectal cancer, non-Hodgkin's lymphoma, etc.).

A native antibody generally includes in its immunoglobulin unit two identical heavy (H) chains and two identical light (L) chains, and thus despite the presence of two antigen-binding sites, such naturally occurring antibody specifically targets only one single antigen. Although such single antigen-targeting (i.e. monospecific) antibody drugs have achieved successes in treating quite a few human diseases, they have failed to achieve significant efficacy in the treatment of many complex diseases, such as cancers, which are commonly driven by multiple factors.

As such, there is a great need to design multispecific antibody drugs that can simultaneously bind to two or more different antigens in one single therapeutic molecule to thereby create additive, complementary, or synergistic effects superior to the effect of individual monospecific antibodies. Such a multispecific antibody could be useful in, for example, bringing the more than the two or more different antigens into close proximity and thereby promoting their interactions. For one example, by bringing immunological cells in close proximity to a tumor-associated antigen or a pathogen antigen can promote the recognition or elimination of tumor or pathogen cells by the immune system. For another, a multispecific antibody can bind to different (and preferably non-overlapping) epitopes of one single antigen, which could be helpful in enhancing the recognition of or binding to a target antigen, in particular one which is susceptible to mutation (e.g. a viral antigen).

Towards this goal, a lot of efforts have been put into designing new formats of immunoglobulins that exhibit multispecificity, mostly notably bispecificity, to simultaneously bind to more than one epitope. Regarding bispecific antibodies, which are the predominant type of multispecific antibody, a variety of bispecific formats have been designed, which can be categorized into IgG-like bispecific antibodies and non-IgG-like bispecific antibodies (e.g., DVD-Ig, CrossMab, BiTE etc.) (Spiess et al., Molecular Immunology, 67(2), pp. 95-106 (2015)). These formats, however, commonly have limitations in specificity, stability, solubility, yield, short half-life, and immunogenicity.

Among these bispecific antibody formats, an IgG-like bispecific antibody is substantially a monoclonal antibody having one Fc region and two Fab arms which specifically target and bind to two different antigens or two different epitopes of a single antigen, respectively. In order to facilitate downstream development, it is desired that such bispecific molecules can be conveniently produced like normal IgG, i.e., produced from a single host cell (e.g. quadroma), with high expression level and purity. However, because the pairing of cognate light-heavy chains as well as the assembly of two different half antibodies typically cannot be automatically controlled, this traditional method has high chance of mispairing resulting in significant product heterogeneity, and thus can be quite inefficient and of poor quality.

Several strategies have been designed for reducing light chain mispairing. In the CrossMab platform developed by Roche, domains of CH1 and CL regions are substantially swapped (Schaefer et al., Proceedings of the National Academy of Sciences of the United States of America, 108(27), pp. 11187-11192 (2011)). In another strategy developed by MedImmune, alternative disulfide bonds are introduced in the CH1 and CL regions (Mazor et al., mAbs, 7(2), pp. 377-389 (2015); U.S. Pat. No. 9,527,927; and US Pub No.: 20180022807A1). In yet another strategy developed by Amgen, new electrostatics is introduced in the CH1-CL region (Liu et al., Journal of Biological Chemistry, 290(12), pp. 7535-7562 (2015); and US Pub No.: 20140154254A1). In yet another strategy developed by Lilly (Lewis et al., Nature Biotechnology, 32(2), pp. 191-198 (2014)) and Genentech (Dillon et al., mAbs, 9(2), pp. 213-230 (2017)), mutations are introduced in both variable and constant domains. However, these existing solutions each has its own disadvantages, and therefore there still exists great needs for better solutions to reduce light chain mispairing.

### BRIEF SUMMARY OF THE INVENTION

The present disclosure provides novel polypeptide complexes that are useful for enhancing correct light chain pairing in a bispecific or multi-specific molecule.

In one aspect, the present disclosure provides a polypeptide complex comprising a first target-binding domain comprising a first target-binding moiety operably linked to a first constant moiety, wherein the first constant moiety comprises a first heavy chain constant region 1 (CH1) associated with a first light chain constant region (CL), wherein the first CH1 region comprises a first amino acid residue at EU position n1, and the first CL region comprises a second amino acid residue at EU position n2, wherein n1:n2 position pair is selected from the group consisting of 128:118 and 173:160, and wherein the first amino acid residue and the second amino acid residue form a covalent bond. In some of these embodiments, the first CH1 region further comprises a third amino acid residue at EU position n3, and the first CL region further comprises a fourth amino acid residue at EU position n4, wherein n3:n4 position pair is selected from the group consisting of 183:176, 141:116, 126:121, and 218:122; wherein the third amino acid residue and the fourth amino acid residue form a non-covalent bond. In some embodiments, the first CH1 region further comprises a third amino acid residue at EU position n3, and the first CL region further comprises a fourth amino acid residue at EU position n4, wherein n3:n4 position pair is selected from the group consisting of 183:176, 141:116, 126:121, and 218:122; wherein the third amino acid residue and the fourth amino acid residue are oppositely charged.

In some embodiments, the first target-binding domain comprises or is an antigen-binding domain. In some embodiments, the first target-binding moiety comprises or is an antigen-binding moiety.

In some embodiments, the polypeptide complex provided herein further comprises a second target-binding domain comprising a second target-binding moiety operably linked to a second constant moiety, wherein the second constant moiety comprises a second CH1 region associated with a second CL region, wherein the first CH1 region does not substantially bind to the second CL region, and the second CH1 region does not substantially bind to the first CL region.

In some embodiments, the second target-binding domain comprises or is an antigen-binding domain. In some embodiments, the second target-binding moiety comprises or is an antigen-binding moiety.

In some embodiments, the second CH1 region comprises a first corresponding amino acid residue at EU position n1′, and the second CL region comprises a second corresponding amino acid residue at EU position n2′, wherein n1′: n2′ position pair is identical to the n1:n2 position pair, and wherein the first corresponding amino acid residue at EU position n1′ does not form a covalent bond with the second amino acid residue at EU position n2, and/or the second corresponding amino acid residue at EU position n2′ does not form a covalent bond with the first amino acid residue at EU position n1.

In some embodiments, the first corresponding amino acid residue at EU position n1′ and the second corresponding amino acid residue at EU position n2′ do not form a covalent bond.

In some embodiments, the second CH1 region further comprises a third corresponding amino acid residue at EU position n3′, and the second CL region further comprises a fourth corresponding amino acid residue at EU position n4′, wherein the n3′:n4′ position pair is identical to the n3:n4 position pair, and wherein:
(a) the fourth corresponding amino acid residue at EU position n4′ and the third amino acid residue at EU position n3 are not oppositely charged or are like-charged, and/or
(b) the third corresponding amino acid residue at EU position n3′ and the fourth amino acid residue at EU position n4 are not oppositely charged or are like-charged.

In some embodiments, the third corresponding amino acid residue at EU position n3′ and/or the fourth corresponding amino acid residue at EU position n4′ are not charged.

In some embodiments, the second CH1 region further comprises a fifth corresponding amino acid residue at EU position n5′, and the second CL region further comprises a sixth corresponding amino acid residue at EU position n6′, and wherein the fifth corresponding amino acid residue and the sixth corresponding amino acid residue form a covalent bond, wherein n5′:n6′ position pair is different from the n1:n2 position pair.

In some embodiments, the n5′:n6′ position pair is selected from the group consisting of 220:214 (for IgG1), 131:214 (for IgG2 and IgG4), 128:118 and 173:160. In some embodiments, the n5′:n6′ position pair is 220:214 (for IgG1). In some embodiments, the n5′:n6′ position pair is 131:214 (for IgG2 and IgG4). In some embodiments, the n5′:n6′ position pair is 128:118, and the n1:n2 position pair is 173:160; or the n5′:n6′ position pair is 173:160, and the n1:n2 position pair is 128:118.

In some of these embodiments, the first CH1 region has an amino acid residue other than cysteine at EU position 220 (for IgG1) or 131 (for IgG2 and IgG4) and the first CL region has an amino acid residue other than cysteine at EU position 214. In some of these embodiments, the second CH1 region has an amino acid residue other than cysteine at EU position 220 (for IgG1) or 131 (for IgG2 and IgG4) and the second CL region has an amino acid residue other than cysteine at EU position 214. In some of these embodiments, neither the first CH1 region nor the second CH1 region has a cysteine residue at EU position 220 (for IgG1) or 131 (for IgG2 and IgG4), and/or neither the first CL region nor the second CL region has a cysteine residue at EU position 214.

In some embodiments, the first CH1 region further comprises a fifth amino acid residue at EU position n5, and the first CL region further comprises a sixth amino acid residue at EU position n6, and wherein n5:n6 position pair is identical to the n5′:n6′ position pair, and wherein the fifth corresponding amino acid residue at EU position n5′ does not form a covalent bond with the sixth amino acid residue at EU position n6, and/or the sixth corresponding amino acid residue at EU position n6′ does not form a covalent bond with the fifth amino acid residue at EU position n5.

In some embodiments, the fifth amino acid residue at EU position n5 and the sixth amino acid residue at EU position n6 do not form a covalent bond.

In some embodiments, the second CH1 region further comprises a seventh corresponding amino acid residue at EU position n7′ and the second CL region further comprises an eighth corresponding amino acid residue at EU position n8′, wherein n7′:n8′ position pair is selected from the group consisting of 183:176, 141:116, 126:121, and 218:122; wherein the seventh corresponding amino acid residue and the eighth corresponding amino acid residue are oppositely charged, and wherein the n7′:n8′ position pair is different from the n3:n4 position pair.

In some embodiments, n7′:n8′ position pair is selected from the group consisting of 183:176, 141:116, and 126:121.

In some embodiments, the n7′:n8′ position pair is 183:176, and the n3:n4 position pair is selected from the group consisting of 141:116, 126:121, and 218:122. In some embodiments, the n7′:n8′ position pair is 141:116, and the n3:n4 position pair is selected from the group consisting of 183:176, 126:121, and 218:122. In some embodiments, the n7′:n8′ position pair is 126:121, and the n3:n4 position pair is selected from the group consisting of 183:176, 141:116, and 218:122. In some embodiments, the n7′:n8′ position pair is 218:122, and the n3:n4 position pair is selected from the group consisting of 183:176, 141:116, and 126:121.

In some embodiments, the first CH1 region further comprises a seventh amino acid residue at EU position n7, and the second CL region further comprises an eighth amino acid residue at EU position n8, wherein the n7:n8 position pair is identical to the n7′:n8′ position pair, and wherein the seventh corresponding amino acid residue at EU position n7′ and the eighth amino acid residue at EU position n8 are not oppositely charged or are like-charged, and/or the eighth corresponding amino acid residue at EU position n8′ and the seventh amino acid residue at EU position n7 are not oppositely charged or are like-charged.

In some embodiments, the seventh amino acid residue at EU position n7 and/or the eighth amino acid residue at EU position n8 are not charged.

Herein, the covalent bond is substantially a chemical bond formed between the first amino acid residue and the second amino acid residue to covalently link the first CH1 region and the first CL region in the polypeptide complex. Such a chemical bond can be a disulfide bond formed between two cysteine residues, yet it is also possible that such a chemical bond is of a different type.

In some embodiments, the covalent bond is a disulfide bond.

In some embodiments, the disulfide bond is formed between two cysteine residues.

In some embodiments, the first amino acid residue at EU position n1 and the second amino acid residue at EU position n2 are both cysteine residues, and/or the fifth corresponding amino acid residue at EU position n5′ and the sixth corresponding amino acid residue at EU position n6′ are both cysteine residues.

In some embodiments, the first CH1 region comprises a substitution of L128C (EU position n1) and the first CL region comprises a substation of F118C (EU position n2). In some of these embodiments, the second CH1 region comprises a substitution of V173C (EU position n5′) and the second CL region comprises a substation of Q160C (EU position n6′) for a kappa light chain or E160C (EU position n6′) for a lambda light chain.

In some embodiments, the first CH1 region comprises a substitution of V173C (EU position n1) and the first CL region comprises a substation of Q160C (EU position n2) for a kappa light chain or E160C (EU position n2) for a lambda light chain. In some of these embodiments, the second CH1 region comprises a substitution of L128C (EU position n5′) and the second CL region comprises a substation of F118C (EU position n6′).

In some embodiments, the third amino acid residue at EU position n3 is a positive-charged amino acid residue, and the fourth amino acid residue at EU position n4 is a negative-charged amino acid residue. In some embodiments, the third amino acid residue at EU position n3 is a negative-charged amino acid residue, and the fourth amino acid residue at EU position n4 is a positive-charged amino acid residue.

In some embodiments, the seventh corresponding amino acid residue at EU position n7′ is a positive-charged amino acid residue, and the eighth corresponding amino acid residue at EU position n8′ is a negative-charged amino acid residue. In some embodiments, the seventh corresponding amino acid residue at EU position n7′ is a negative-charged amino acid residue, and the eighth corresponding amino acid residue at EU position n8′ is a positive-charged amino acid residue.

In some embodiments, the positive-charged amino acid residue is selected from the group consisting of lysine (K), histidine (H) and arginine (R), and/or the negative-charged amino acid residue is selected from the group consisting of aspartic acid (D) and glutamic acid (E).

In some embodiments, at least one, two, three, or four of the first amino acid residue at EU position n1, the second amino acid residue at EU position n2, the third amino acid residue at EU position n3, and fourth amino acid residue at EU position n4 are introduced by substitution.

In some embodiments, the third amino acid residue and the fourth amino acid residue at the n3:n4 position pair are substitutions selected from the group consisting of: S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R.

In some embodiments, at least one, two, three, or four of the fifth corresponding amino acid residue at EU position n5′, the sixth corresponding amino acid residue at EU position n6′, the seventh corresponding amino acid residue at EU position n7′, and the eighth corresponding amino acid residue at EU position n8′ are introduced by substitution.

In some embodiments, the seventh corresponding amino acid residue and the eighth corresponding amino acid residue at the n7′:n8′ position pair are substitutions selected from the group consisting of: S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R, and wherein the n7′:n8′ position pair is different from the n3:n4 position pair.

In some embodiments, the first target-binding domain comprises a first combination of substitutions at (n1+n2):(n3+n4) positions, and/or the second target-binding domain comprises a second combination of substitutions at (n5′+n6′):(n7′+n8′) positions, and wherein the first combination of substitutions and/or the second combination of substitutions are selected from the group consisting of: (L128C+S183K):(F118C+S176D), (L128C+S183K):(F118C+S176E), (L128C+S183R):(F118C+S176D), (L128C+S183R):(F118C+S176E), (L128C+S183H):(F118C+S176D), (L128C+S183H):(F118C+S176E), (L128C+S183D):(F118C+S176K), (L128C+S183D):(F118C+S176R), (L128C+S183D):(F118C+S176H), (L128C+S183E):(F118C+S176K), (L128C+S183E):(F118C+S176R), (L128C+S183E):(F118C+S176H), (V173C+A141K):(Q160C (or E160C)+F116D), (V173C+A141K):(Q160C (or E160C)+F116E), (V173C+A141R):(Q160C (or E160C)+F116D), (V173C+A141R):(Q160C (or E160C)+F116E), (V173C+A141H):(Q160C (or E160C)+F116D), (V173C+A141H):(Q160C (or E160C)+F116E), (V173C+A141D):(Q160C (or E160C)+F116K), (V173C+A141D):(Q160C (or E160C)+F116R), (V173C+A141D):(Q160C (or E160C)+F116H), (V173C+A141E):(Q160C (or E160C)+F116K), (V173C+A141E):(Q160C (or E160C)+F116R), (V173C+A141E):(Q160C (or E160C)+F116H), (V173C+S183K):(Q160C (or E160C)+S176D), (V173C+S183K):(Q160C (or E160C)+S176E), (V173C+S183R):(Q160C (or E160C)+S176D), (V173C+S183R):(Q160C (or E160C)+S176E), (V173C+S183H):(Q160C (or E160C)+S176D), (V173C+S183H):(Q160C (or E160C)+S176E), (V173C+S183D):(Q160C (or E160C)+S176K), (V173C+S183D):(Q160C (or E160C)+S176R), (V173C+S183D):(Q160C (or E160C)+S176H), (V173C+S183E):(Q160C (or E160C)+S176K), (V173C+S183E):(Q160C (or E160C)+S176R), (V173C+S183E):(Q160C (or E160C)+S176H), (L128C+F126K):(F118C+S121D), (L128C+F126K):(F118C+S121E), (L128C+F126R):(F118C+S121D), (L128C+F126R):(F118C+S121E), (L128C+F126H):(F118C+S121D), (L128C+F126H):(F118C+S121E), (L128C+F126D):(F118C+S121K), (L128C+F126D):(F118C+S121R), (L128C+F126D):(F118C+S121H), (L128C+F126E):(F118C+S121K), (L128C+F126E):(F118C+S121R), (L128C+F126E):(F118C+S121H), (V173C+F126K):(Q160C (or E160C)+S121D), (V173C+F126K):(Q160C (or E160C)+S121E), (V173C+F126R):(Q160C (or E160C)+S121D), (V173C+F126R):(Q160C (or E160C)+S121E), (V173C+F126H):(Q160C (or E160C)+S121D), (V173C+F126H):(Q160C (or E160C)+S121E), (V173C+F126D):(Q160C (or E160C)+S121K), (V173C+F126D):(Q160C (or E160C)+S121R), (V173C+F126D):(Q160C (or E160C)+S121H), (V173C+F126E):(Q160C (or E160C)+S121K), (V173C+F126E):(Q160C (or E160C)+S121R), (V173C+F126E):(Q160C (or E160C)+S121H), (L128C+K218D):(F118C+D122K), (L128C+K218D):(F118C+D122H), (L128C+K218D):(F118C+D122R), (L128C+K218E):(F118C+D122K), (L128C+K218E):(F118C+D122H), (L128C+K218E):(F118C+D122R), (V173C+K218D):(Q160C (or E160C)+D122K), (V173C+K218D):(Q160C (or E160C)+D122H), (V173C+K218D):(Q160C (or E160C)+D122R), (V173C+K218E):(Q160C (or E160C)+D122K), (V173C+K218E):(Q160C (or E160C)+D122H), and (V173C+K218E):(Q160C (or E160C)+D122R), provided that, when both the first combination of substitutions and the second combination of substitutions are selected, the n5′:n6′ position pair is different from the n1:n2 position pair, and the n7′:n8′ position pair is different from the n3:n4 position pair.

In some embodiments, the first target-binding moiety comprises a first polypeptide fragment operably linked to the first CL region, and the second target-binding moiety comprises a second polypeptide fragment operably linked to the second CL region, wherein the first polypeptide fragment has a different amino acid sequence from the second polypeptide fragment. In some embodiments, either the first polypeptide fragment or the second polypeptide fragment is absent from the polypeptide complex.

In some embodiments, the polypeptide complex can be a fusion protein that comprises two or more polypeptide fragments operably linked to a CH1 region and a CL region, respectively. In some embodiments, the first target-binding moiety further comprises a third polypeptide fragment operably linked to the first CH1 region, and the second target-binding moiety comprises a fourth polypeptide fragment operably linked to the second CH1 region. In some embodiments, the third polypeptide fragment has a different amino acid sequence from the fourth polypeptide fragment. In some embodiments, either the third polypeptide fragment or the fourth polypeptide fragment is absent from the polypeptide complex.

In some embodiments, the first polypeptide fragment and the third polypeptide fragment can each contain a target-binding site and bind to its target molecule. For example, the first polypeptide fragment and the third polypeptide fragment can bind to the same target molecule, or alternatively bind to different target molecules. For another example, the first polypeptide fragment and the third polypeptide fragment can have either identical or different amino acid sequences. In some embodiments, either the first polypeptide fragment or the third polypeptide fragment is absent from the polypeptide complex.

Likewise, the second polypeptide fragment and the fourth polypeptide fragment can each contain a target-binding site and bind to its target molecule. For example, the second polypeptide fragment and the fourth polypeptide fragment can bind to the same target molecule, or alternatively binds to different target molecules. For another example, the second polypeptide fragment and the fourth polypeptide fragment can have either identical or different amino acid sequences. In some embodiments, either the second polypeptide fragment or the fourth polypeptide fragment is absent from the polypeptide complex. In some embodiments, one, two or three of the four polypeptide fragments are absent from the polypeptide complex.

In some embodiments, the first polypeptide fragment and the third polypeptide fragment can be associated to form a first target-binding site. Likewise, the second polypeptide fragment and the fourth polypeptide fragment can be associated to form a second target-binding site. In some embodiments, the first target-binding site and the second target-binding site can bind to the same target molecule, or different parts on the same target molecule, or different target molecules.

In some embodiments, the first polypeptide fragment and the third polypeptide fragment each contains a first target-binding site or associate with each other to form a first target-binding site; and/or the second polypeptide fragment and the fourth polypeptide fragment each contains a second target-binding site or associate with each other to form a second target-binding site.

In some embodiments, the first target-binding moiety can be a first antigen-binding moiety and/or the second target-binding moiety can be a second antigen-binding moiety. In some embodiments, the antigen-binding moiety is derived from one or more antibody fragments.

In some embodiments, the first antigen-binding moiety can comprise a first VL region and a first VH region, which are associated to form a first antigen-binding site. In some embodiments, the second antigen-binding moiety can comprise a second VL region and a second VH region, which are associated to form a second antigen-binding site. The first antigen-binding site and the second antigen-binding site may bind to the same antigen, or different epitopes on the same antigen, or different antigens.

In some embodiments, the first antigen-binding domain and/or the second antigen-binding domain is contained within an antibody, optionally a bispecific antibody or a multispecific antibody.

In some embodiments, the second antigen-binding domain and the first antigen-binding domain bind to different antigens or bind to different epitopes on the same antigen.

In some embodiments, wherein:
(a) one of the first antigen-binding domain and the second antigen-binding domain binds to a tumor-associated antigen, and the other binds to an immune related target; or
(b) one of the first antigen-binding domain and the second antigen-binding domain binds to a first tumor-associated antigen, and the other binds to a second tumor-associated antigen.

In some embodiments, the first and/or the second antigen-binding domain is chimeric, humanized, or fully human.

In some embodiments, the first and/or the second antigen-binding moiety is selected from the group consisting of a nanobody, an Fv fragment, a scFv, a disulfide stabilized Fv fragment, a (dsFv)₂, a bispecific dsFv, and a diabody.

In some embodiments, the first and/or the second antigen-binding domain is selected from the group consisting of a Fab domain, a Fab′, and a F(ab′)₂.

In some embodiments, the first antigen-binding domain and/or the second antigen-binding domain comprises one or more CDRs operably linked to a CH1 region and a CL region.

In some embodiments, the first antigen-binding domain is a first Fab domain, and/or the second antigen-binding domain is a second Fab domain.

Herein, the polypeptide complex can be an antibody or a fragment thereof having a Fab domain. Examples of the polypeptide complex can include, without any limitation on the scope of the disclosure, a monospecific antibody, a bispecific antibody, a trifunctional antibody, a Fab, a Fab′, a F(ab′)₂, etc. The polypeptide complex can be expanded to any other molecule that contains a Fab domain/region.

In some embodiments, the second Fab domain comprises:
(a) one or more light chain CDRs and/or light chain framework regions different from that of the first Fab domain; and optionally,
(b) one or more heavy chain CDRs and/or heavy chain framework regions different from that of the first Fab domain.

In some embodiments, the polypeptide complex further comprises an Fc region operably linked to the first target-binding domain and the second target-binding domain.

In some embodiments, the Fc region is derived from IgG, IgA, IgM, IgE or IgD. In some embodiments, the Fc region is derived from IgG. In some embodiments, the Fc region is derived from IgG1, IgG2, IgG3 or IgG4. In some embodiments, the Fc region is derived from IgG1.

In some embodiments, the Fc region is heterodimeric.

In some embodiments, the heterodimeric Fc region comprises one or more mutations that facilitate heterodimerization.

In some embodiments, the heterodimeric Fc region comprises a first Fc polypeptide comprising a first Fc mutation, and/or a second Fc polypeptide comprising a second Fc mutation, wherein:
a) the first Fc mutations comprises T366W or S354C, and the second Fc mutation comprises Y349C, T366S, L368A, or Y407V;
b) the first Fc mutation comprises D399K or E356K, and the second Fc mutation comprises K392D, or K409D;
c) the first Fc mutation comprises E356K, E357K, or D399K, and the second Fc mutation comprises K370E, K409D, or K439E;
d) the first Fc mutation comprises S364H, or F405A, and the second Fc mutation comprises Y349T, or T394F;
e) the first Fc mutation comprises S364H, or T394F, and the second Fc mutation comprises Y394T, or F405A;
f) the first Fc mutation comprises K370D, or K409D, and the second Fc mutation comprises E357K, or D399K; or
g) the first Fc mutation comprises L351D, or L368E, and the second Fc mutation comprises L351K, or T366K,
   wherein numbering is according to the EU index.

In another aspect, the present disclosure provides a nucleic acid comprising a nucleotide sequence encoding the polypeptide complex provided herein or a part thereof.

In another aspect, the present disclosure provides a vector comprising the nucleic acid provided herein.

In another aspect, the present disclosure provides a host cell comprising the nucleic acid provided herein or the vector provided herein.

In another aspect, the present disclosure provides a pharmaceutical composition comprising the polypeptide complex provided herein and a pharmaceutically acceptable carrier.

In another aspect, the present disclosure provides a conjugate comprising the polypeptide complex provided herein and a payload conjugated thereto, wherein the payload is selected from the group consisting of a radioactive label, a fluorescent label, an enzyme-substrate label, an affinity purification tag, a tracer molecule, an anticancer drug, and a cytotoxic molecule.

In another aspect, the present disclosure provides a composition comprising the polypeptide complex provided herein, or the conjugate provided herein, and a pharmaceutically acceptable carrier.

In another aspect, the present disclosure provides a method of treating or preventing from a disease, condition, or symptom. The method comprises administering to a subject in need thereof a therapeutically effective amount of the polypeptide complex provided herein, the pharmaceutical composition provided herein, the conjugate provided herein, or the composition provided herein.

In some embodiments, the disease is selected from the group consisting of a cancer, an inflammatory disease, an infectious or parasitic disease, a cardiovascular disease, neuropathy, a neuropsychiatric condition, an injury, an autoimmune disease, a metabolic disease, a neurodegenerative disease, or a coagulation disorder.

In another aspect, the present disclosure provides a method of detecting presence or level of an antigen, comprising contacting a sample suspected of containing the antigen with the polypeptide complex provided herein, and determining the formation of a complex between the antigen and the polypeptide complex.

The foregoing and other features of the invention will become more apparent from the following detailed description of several embodiments which proceeds with reference to the accompanying figures.

### BRIEF DESCRIPTION OF FIGURES

FIG. 1 shows the SDS-PAGE results of DIC002 (FIG. 1A), DIC007 (FIG. 1B) and DIC014 (FIG. 1C).

FIG. 2 shows the SEC-HPLC results of DIC002 (FIG. 2A), DIC007 (FIG. 2B) and DIC014 (FIG. 2C).

FIG. 3 shows the LC-MS results of intact DIC014 (FIG. 3A) and deglycosylated DIC014 (FIG. 3B).

FIG. 4 shows the SDS-PAGE results of DIC003 (FIG. 4A), DIC004 (FIG. 4B), DIC005 (FIG. 4C), DIC006 (FIG. 4D), DIC009 (FIG. 4E) and DIC010 (FIG. 4F).

FIG. 5 shows the SEC-HPLC results of DIC003 (FIG. 5A), DIC004 (FIG. 5B), DIC005 (FIG. 5C), DIC006 (FIG. 5D), DIC009 (FIG. 5E) and DIC010 (FIG. 5F).

FIG. 6 shows the SDS-PAGE results of DIC015 (FIG. 6A) and DIC016 (FIG. 6B).

FIG. 7 shows the SEC-HPLC results of DIC015 (FIG. 7A) and DIC016 (FIG. 7B).

FIG. 8 shows the LC-MS results of intact DIC015 (FIG. 8A) and deglycosylated DIC015 (FIG. 8B).

FIG. 9 shows the LC-MS results of intact DIC016 (FIG. 9A) and deglycosylated DIC016 (FIG. 9B).

FIG. 10 shows the LC-MS results of intact DIC009 (FIG. 10A) and deglycosylated DIC009 (FIG. 10B). FIG. 10C shows partial enlarged FIG. 10B.

FIG. 11 shows the LC-MS results of intact DIC010 (FIG. 11A) and deglycosylated DIC010 (FIG. 11B). FIG. 11C shows partial enlarged FIG. 11B.

FIG. 12 shows the binding affinities of DIC010 to human PD1 (FIG. 12A), human TGFβR2 (FIG. 12B), and human TGFβR3 (FIG. 12C), as measured by SPR assay.

FIG. 13 shows the efficacy of different dosages (0 mg/kg, 1 mg/kg, 3 mg/kg and 10 mg/kg) of DIC010 on human PD1-MC38 syngeneic mice models.

FIG. 14 shows the binding area analysis of CH-CL interface.

FIG. 15 shows the binding area analysis of CH1-VH and CL-VL interfaces.

FIG. 16 shows the amino acid residues that are selected for mutation study.

### DETAILED DESCRIPTION OF THE INVENTION

Provided herein include polypeptide complexes having an engineered target-binding domain, and bispecific antibodies engineered to have high selectivity for immunoglobulin light chain-heavy chain cognate pairing.

Before the detailed description of the inventions is provided, the following are noted and defined.

All the description provided herein is merely intended to illustrate various embodiments of the inventions provided in the present disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein.

All references cited in the present disclosure, including patent applications, issued patents, published articles or other publications, are incorporated by reference in their entirety, which are for the purpose of providing methodologies that might be used in connection with the description provided herein. With respect to any term that is presented in one or more publications that is similar to, or identical with, a term that has been expressly defined in this disclosure, the definition of the term as expressly provided in this present disclosure will control in all respects.

All technical and scientific terms used, unless expressly defined otherwise, in this present disclosure, are generally deemed to have the same meaning as commonly understood to one of ordinary skill in the art to which this disclosure belongs.

As used herein, i.e., throughout the whole disclosure, the articles “a,” “an,” and “the” are to be construed to mean “one or more” or “at least one” unless specified otherwise. By way of example, “a polypeptide complex” means one polypeptide complex or more than one polypeptide complex.

As used herein, the terms “about,” “approximately,” “around” or alike, refer to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “comprise,” “comprises,” “comprising,” “include,” “includes,” “including,” “contain,” “contains,” “containing”, “have,” “has,” “having” and the like, are synonymous and are used inclusively, in an open-ended fashion, and do not exclude additional elements, features, steps, acts, operations, and so forth.

As used herein, the term “or” is used in its inclusive sense (and not in its exclusive sense) so that when used, for example, to connect a list of elements, the term “or” means one, some, or all of the elements in the list.

As used herein, the phrase “at least one” means one or more, i.e. 1, 2, 3, 4, 5, 6, 7, 8, 9, 10, or more. A phrase referring to “at least one of” a list of items is construed to refer to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiment requires at least one of X, at least one of Y, and at least one of Z to each be present.

As used herein, the references “one embodiment,” “an embodiment,” “a particular embodiment,” “a related embodiment,” “a certain embodiment,” “an additional embodiment,” “some embodiments,” “certain embodiments,” or “a further embodiment” or combinations thereof, are to be understood to mean that a particular feature, structure or characteristic described in connection with this particular embodiment is included in at least one embodiment of the present disclosure. Thus, the presences or appearances of the foregoing phrases in various places throughout this disclosure are not necessarily all referring to the same embodiment. Furthermore, the particular features, structures, or characteristics may be combined in any suitable manner in one or more embodiments.

Conditional language used herein, such as, among others, “can,” “could,” “might,” “may,” “e.g.,” and the like, unless stated expressly otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

### I. Terminology and Definitions

In this section, definitions for some general terms are provided. Definition for other terms may be found in other sections of the disclosure that follow.

As used herein, and throughout other parts of the present disclosure as well, the term “polypeptide” is used interchangeably to “peptide,” “protein,” or alike, and is used to refer to a polymer of amino acid residues, or an assembly of multiple polymers of amino acid residues. The term applies to both a naturally occurring amino acid polymer and a non-naturally occurring amino acid polymer, and is construed to also cover an amino acid polymer in which one or more amino acid residues are artificial or synthetic chemical mimetics of their corresponding naturally occurring amino acids. The term “protein” typically refers to large polypeptides. The term “peptide” typically refers to short polypeptides. Polypeptide sequences are usually described as the left-hand end of a polypeptide sequence is the amino-terminus (N-terminus), and the right-hand end of a polypeptide sequence is the carboxyl-terminus (C-terminus).

As used herein, the term “polypeptide complex,” “protein complex,” or alike, refers to a complex comprising one or more polypeptides/protein subunits that are associated with one another that can perform certain functions, for example to specifically recognize and bind to certain target antigens.

As used herein, the term “amino acid” refers to a building block of a protein, a peptide, a polypeptide or an amino acid polymer, and the term “amino acid” further refers to a naturally occurring or synthetic amino acid, as well as any amino acid analog and amino acid mimetics that functions in a manner similar to the naturally occurring amino acid. Naturally occurring amino acids are those encoded by the genetic codes, as well as those amino acids that are later modified, e.g., hydroxyproline, gamma-carboxyglutamate, and O-phosphoserine. As used within this application, naturally occurring amino acids include the group of naturally occurring carboxy alpha-amino acids comprising alanine (three letter code: Ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). Herein, “amino acid analogs” refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters. Herein, “amino acid mimetics” refer to chemical compounds that have a structure that is different from the general chemical structure of an amino acid, but that functions in a manner similar to a naturally occurring amino acid.

As used herein, the term “nucleic acid,” “nucleic acid molecule,” “nucleotide,” “polynucleotide” or alike, is construed to refer to a nucleotide polymer of any length, and can include both DNA and RNA, and can be single-stranded or double-stranded. Herein, nucleotides in a nucleic acid can include deoxyribonucleotides, ribonucleotides, modified nucleotides (e.g., methylated nucleotides) or bases, or analogs thereof. The nucleic acid or polynucleotide are typically obtained by polymerization by means of a DNA or a RNA polymerase, or by means of a synthetic reaction. Herein, the terms also refer to synthetic and/or non-naturally occurring nucleic acid molecules (e.g., comprising nucleotide analogues or modified backbone residues or linkages). The terms encompass nucleic acids containing analogues of natural nucleotides. The terms also encompass nucleic acid-like structures with synthetic backbones. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “antibody” as used herein encompasses any immunoglobulin, monoclonal antibody, polyclonal antibody, multispecific antibody, bispecific antibody, bivalent antibody or multivalent antibody that binds to a specific antigen. In the following, a description of an antibody, as well as terms relevant thereto, is provided in more detail.

In mammals such as human, depending on the different types of heavy chains present in the immunoglobulins, there are five different classes/isotypes (i.e. IgA, IgD, IgE, IgG, and IgM, corresponding to the five Ig heavy chain types α, δ, ε, γ, and μ, respectively) of antibodies, which typically have different molecular and biological properties, functional locations, physiological functionalities, and pathological implications in diseases. Certain antibody classes may further include subclasses. For example, in human, IgA may include IgA1 and IgA2 subclasses, and IgG may include four subclasses denoted as IgG1, IgG2, IgG3, and IgG4, respectively. With an immunoglobulin monomer as its basic functional unit, a mammalian antibody may exist as a monomer (e.g. IgD, IgE, and IgG), a dimer (IgA), a tetramer (IgM), or a pentamer (IgM). In mammals, two types of light chain exist, including kappa (x) chain and lambda (k) chain.

Within the basic immunoglobulin unit, a native or naturally occurring antibody such as IgG generally includes two identical heavy (H) chains and two identical light (L) chains. Each light chain is linked to a heavy chain by one covalent disulfide bond or linkage formed between a pair of cysteine residues present respectively in the each light chain and the heavy chain, and the two heavy chains are further linked to one another through several disulfide bonds formed between cysteine residues in each heavy chain. The tetramer thus formed substantially takes a Y-like shape for the antibody, with the end of each fork arm containing an identical antigen-binding site (i.e. paratope) that interacts specifically with a corresponding epitope of the antigen.

As used herein, the term “domain” refers to a globular structure formed by one or more regions of one or more polypeptide chains comprising peptide loops (e.g., comprising 3 to 4 peptide loops) that are stabilized, for example, by β-pleated sheet and/or intrachain disulfide bond(s). Examples may include a Fab domain (see below for more details). It is noted that in the present disclosure, the two terms “domain” and “region” may be used interchangeably.

More specifically, in a native antibody, in a N-terminal-to-C-terminal direction, each heavy chain includes a variable region (VH, or HCVR), followed by three or four constant regions (“CHs”, with IgA, IgD, IgG containing three CH regions CH1, CH2 and CH3; and IgE and IgM containing four CH regions CH1, CH2, CH3 and CH4), and each light chain includes a variable region (VL, or LCVR) and a constant region (CL). In the Y-shaped antibody, the variable region of each light chain (i.e. the VL region) aligns or associates with the variable region of its pairing heavy chain (i.e. the VH region) to together form an antigen-binding site for the antibody.

The term “variable region” or “VR” as used herein means the region in heavy chain or light chain of an antibody that are responsible for antigen binding. In a native antibody, the heavy chain variable region (VH or HCVR) contains three highly variable loops called “complementarity determining regions” (CDRs), i.e., heavy (H) chain CDRs including HCDR1, HCDR2, HCDR3, and the light chain variable region (VL or LCVR) contains three light (L) chain CDRs including LCDR1, LCDR2, and LCDR3. CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol. Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as “framework regions” (FRs), which are more highly conserved than the CDRs and form a scaffold to support the hypervariable loops. In a native antibody, each VH and VL comprises four FRs, and the CDRs and FRs are arranged from amino terminus to carboxyl terminus in the order: FR1, CDR1, FR2, CDR2, FR3, CDR3, FR4. However, it should be understood that the term “variable region” as used herein does not necessarily need to include all of the three CDRs or all of the four FRs, and should be construed to encompass any variant or derivative of a native variable region from a native antibody, as long as such variant or derivative retains antigen-binding activity.

The term “variant” with respect to a polypeptide or a polynucleotide encompasses all kinds of different forms of the polypeptide or polynucleotide, including without limitation, fragments, mutants, fusions, derivatives, mimetics, or any combination thereof, of the polypeptide or polynucleotide.

The term “derivative” with respect to a polypeptide or a polynucleotide refers to a chemically modified polypeptide or polynucleotide, in which one or more well-defined number of substituent groups have been covalently attached to one or more specific amino acid residues of the polypeptide or one or more specific nucleotides of the polynucleotide. Exemplary chemical modification to polypeptide can be, e.g. alkylation, acylation, esterification, amidation, phosphorylation, glycosylation, labeling, methylation of one or more amino acids, or conjugation with one or more moieties. Exemplary chemical modification to polynucleotide can be (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages.

The term “constant region” or “constant moiety” as used herein means the region in heavy chain or light chain of an antibody that are not directly involved in antigen binding. It should be understood that the term “constant region” or “constant moiety” as used herein does not necessarily need to include the full length of a native constant region of a native antibody, and should be construed to encompass any variant or derivative of such a native constant region or constant moiety, as long as such variant or derivative retains the ability to, for example, support stability of the antigen-binding domain, or retains the intended biological function such as effector functions such as secretion, transplacental mobility, Fc receptor binding, complement binding, and the like.

The term “CL region” refers to the constant region of an immunoglobulin light chain that is adjacent to the VL region. CL region may span from about EU position 108 to about EU position 216 in an immunoglobulin light chain. In a native antibody, the constant region of each light chain (i.e. the CL region) associates with the first constant region of a pairing heavy chain (i.e. the CH1 region).

The term “CH1 region” as used herein encompasses the first (most amino terminal) constant region of an immunoglobulin heavy chain that extends from, about EU position 118 to at least about EU position 220 (e.g. can be extended to EU position 221, and so on). The CH1 region is adjacent to the VH region, and is amino terminal to the hinge region of an immunoglobulin heavy chain molecule.

The term “hinge region” in terms of an antibody includes the portion of a heavy chain molecule that joins the CH1 region to the CH2 region. The length of hinge region may vary depending on the defined boundaries of the CH1 region and of the CH2 region. The hinge region is normally flexible, thus allowing the two N-terminus antigen binding regions to move independently.

The term “CH2 region” as used herein refers to the portion of a heavy chain immunoglobulin molecule that extends, e.g., from about EU position 231 to EU position 340.

The term “CH3 region” as used herein refers to the portion of a heavy chain immunoglobulin molecule that extends approximately 110 residues from N-terminus of the CH2 domain, e.g., from about EU position 341 to EU position 445, 446 or 447. The CH3 domain typically forms the C-terminal portion of the antibody like IgG, IgA, and IgD. In some immunoglobulins like IgE and IgM, however, additional domains may extend from CH3 domain to form the C-terminal portion of the molecule (e.g. the CH4 domain in the chain of IgM and the F chain of IgE).

Throughout the disclosure, the numbers indicating the positions of amino acid residues in a constant region of an antibody, such as those in the heavy chain constant region 1 (CH1) and the light chain constant region (CL) in a constant moiety, are based on the EU numbering system or the EU index, as described in Edelman, G. M. et al., Proc. Natl. Acad. USA, 63, 78-85 (1969); and in Kabat et al., Sequences of Proteins of Immunological Interest, 5th Ed. Public Health Service, National Institutes of Health, Bethesda, Md. (1991). The EU numbering is also available from the IMGT scientific chart, accessible from the website of international ImMunoGeneTics information system.

“Fab” as used herein refers to a single antigen-binding domain derived from an antibody, where the domain has a single heavy chain fragment associated with a single light chain fragment via one or more covalent bonds which are non-peptide bonds. In some embodiments, the single heavy chain fragment in the Fab domain comprises an HCVR and a CH1 region. In some embodiments, the single light chain fragment in the Fab domain comprises an LCVR and a CL domain. In some embodiments, the CH1 region associates with the HCVR by a covalent bond such as a disulfide bond. In a native antibody, the Fab domain corresponds substantially to one arm of the antibody, typically retains the ability to recognize and bind to its corresponding antigen.

“Fc” as used herein refers to a portion derived from an antibody consisting of, in an example of IgG, the second (CH2) and third (CH3) constant regions of a first heavy chain bound to the second and third constant regions of a second heavy chain via one or more covalent bonds which are non-peptide bonds, for example, via disulfide bonding. The Fc portion of the antibody is responsible for various effector functions such as antibody-dependent cellular cytotoxicity (ADCC) and complement-dependent cytotoxicity (CDC), etc., but does not function in antigen binding.

An “antigen” or “Ag” as used herein refers to a compound, composition, peptide, polypeptide, protein or substance (e.g., polypeptide, carbohydrate, nucleic acid, lipid, or other naturally occurring or synthetic compound) that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens. Antigens can include exogenous antigen, endogenous antigen, autoantigen, neoantigen, and antigens associated with certain pathogens or diseases. An exogenous antigen enters a body by inhalation, ingestion or injection, and can be presented by the antigen-presenting cells (APCs) by endocytosis or phagocytosis and form MHC II complex. An endogenous antigen is generated within normal cells as a result of cell metabolism, intracellular viral or bacterial infection, which can form MHC I complex. An autoantigen (e.g. peptide, DNA or RNA, etc.) is recognized by the immune system of a patient suffering from autoimmune diseases, whereas under normal condition, this antigen should not be the target of the immune system. A neoantigen is entirely absent from the normal body, and is generated because of a certain disease, such as tumor or cancer. In certain embodiments, the antigen is associated with a certain disease (e.g. tumor or cancer, autoimmune diseases, infectious and parasitic diseases, cardiovascular diseases, neuropathies, neuropsychiatric conditions, injuries, inflammations, coagulation disorder). In certain embodiments, the antigen is associated with immune system (e.g. immunological cells such as B cell, T cell, NK cells, macrophages, etc.).

An “epitope” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation.

As used herein, the term “antibody fragment” refers to a portion of a full-length antibody, generally the antigen-binding fragment or variable region thereof. Examples of an antibody fragment include Fab, Fab′, F(ab′)₂, and Fv fragments, diabodies, linear antibodies, single-chain antibody molecules, and multispecific antibodies formed from antibody fragments, and most, if not all, of the definitions of these above indicated antibody fragments are further provided below.

As used herein, the term “antigen-binding fragment” or alike, is referred to as an antibody fragment formed from a portion of an antibody comprising one or more CDRs, or any other antibody fragment that binds to an antigen but does not comprise an intact native antibody structure. Examples of antigen-binding fragments may include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody, etc. An antigen-binding fragment is capable of binding to the same antigen to which the parent antibody binds. An antigen-binding fragment may comprise one or more CDRs from a particular human antibody grafted to a framework region from one or more different human antibodies. For more and detailed formats of antigen-binding moiety are described in Spiess et al., 2015 (Supra), and Brinkman et al., mAbs, 9(2), pp. 182-212 (2017), which are incorporated herein by entirety reference.

As used herein, the term “target-binding moiety” refers to a protein or polypeptide fragment or domain that is capable of binding to a target molecule, which may be a chemical or biological entity of any type. For example, the target molecule may be a small molecule compound or a macromolecule such as a peptide, a polypeptide, a protein, or a nucleic acid. The target molecule can be a disease related molecule such as tumor surface antigen, immune checkpoint molecules, cell surface receptors, infectious agents, cytokines, growth factors, and so on. The skilled person can readily select from a wide variety of targets of interest. In some embodiments, the target-binding moiety of the present disclosure comprises or is an antigen-binding moiety.

As used herein, the term “antigen-binding moiety” means the moiety that is responsible for antigen-binding in an antigen-binding domain. For example, the antigen-binding moiety can comprise one or more CDRs, but does not necessarily comprise a constant region/constant moiety. Examples of antigen-binding moieties may include, without limitation, a variable domain, a variable region, a nanobody, an Fv fragment, a scFv, a disulfide stabilized Fv fragment, a (dsFv)₂, a bispecific dsFv, or a diabody.

As used herein, the term “target-binding domain” refers to a domain that comprises a target-binding moiety and a constant moiety comprising a CH1 region and a CL region. For example, the target-binding domain may be a fusion protein comprising a target-binding moiety and a constant moiety. In some embodiments, the target-binding domain of the present disclosure comprises or is an antigen-binding domain.

As used herein, the term “antigen-binding domain” is referred to an antigen-binding fragment that comprises an antigen-binding moiety and a constant moiety. Examples of antigen-binding domains may include, without limitation, a Fab, a Fab′, or a F(ab′)₂.

“Fcab” refers to an engineered Fc fragment that also contains an antigen-binding site. An Fcab can exist as a fragment or can be inserted to a full immunoglobulin by swapping the Fc region to thereby obtain an antibody with bispecific or even trispecific activity.

A “fragment difficult (Fd)” with regard to an antibody refers to the amino-terminal half of the heavy chain fragment that can be combined with the light chain to form Fab.

“Fv” with regard to an antibody refers to the smallest fragment of the antibody to bear the complete antigen-binding site. An Fv fragment consists of the variable domain of a single light chain bound to the variable domain of a single heavy chain. A number of Fv designs have been provided, including dsFvs, in which the association between the two domains is enhanced by an introduced disulfide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerized to form diabodies and triabodies (Maynard et al., Annu Rev Biomed Eng 2 339-376 (2000)).

A “CrossMab” refers to a technology of pairing of unmodified light chain with the corresponding unmodified heavy chain and pairing of the modified light chain with the corresponding modified heavy chain, thus resulting an antibody with reduced mispairing in the light chain.

A “BiTE®” is a bispecific T-cell engager molecule, comprising a first scFv with a first antigen specificity in the LCVR-HCVR orientation linked to a second scFv with a second specificity in the HCVR-LCVR orientation.

As used herein, the term “multispecific antibody” refers to an artificial or engineered antibody that can simultaneously bind to at least two different epitopes. A bispecific antibody is substantially a type of a multispecific antibody. In addition, other multispecific antibodies may include trispecific antibodies, which have three different antigen-binding specificities, tetraspecific antibodies, which have four different antigen-binding specificities, and so on.

As used herein, the term “bispecific antibody” refers to an antibody that comprises two physically separable antigen-binding moieties/sites which differ from one another in their antigen specificity. Usually, a bispecific antibody is an artificial antibody which has fragments derived from two different monoclonal antibodies and is capable of binding to two different epitopes. The two epitopes may present on the same antigen, or they may present on two different antigens. It is in contrast to a naturally occurring antibody which has two physically separable antigen-binding moieties that are structurally identical and thus have the same antigen specificity. In the heterodimeric antibody disclosed herein, each of the two different Fab domains (i.e. the first Fab domain and the second Fab domain) includes a different antigen-binding moiety that binds specifically to a different epitope, and typically includes an immunoglobulin heavy chain variable region (VH) and an immunoglobulin light chain variable region (VL), which differ in sequence from each other. In an effort to increase the chance for proper cognate pairing to obtain the bispecific antibodies in a highly selective and efficient manner, there have been several strategies for facilitating the light chain-heavy chain cognate pairing and the heavy chain-heavy chain cognate pairing.

The term “affinity” as used herein refers to the strength of non-covalent interaction between an immunoglobulin molecule (i.e. antibody) or fragment thereof and an antigen.

### II. Polypeptide Complexes

The present disclosure provides novel polypeptide complexes that are useful for facilitating selective pairing (i.e. cognate pairing) of a light chain and a heavy chain in a construct containing at least two different light chains that associate with the respective heavy chain(s) via one or more non-peptide bonds.

In one aspect, the present disclosure provides a polypeptide complex comprising a first target-binding domain comprising a first target-binding moiety operably linked to a first constant moiety, wherein the first constant moiety comprises a first heavy chain constant region 1 (CH1) associated with a first light chain constant region (CL), wherein the first CH1 region comprises a first amino acid residue at EU position n1, and the first CL region comprises a second amino acid residue at EU position n2, wherein n1:n2 position pair is selected from the group consisting of 128:118 and 173:160, and wherein the first amino acid residue and the second amino acid residue form a covalent bond.

As used herein, the term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, a target-binding moiety may be operably linked to a constant moiety so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

In certain embodiments, the linker as used herein is selected from the group consisting of a cleavable linker, a non-cleavable linker, a peptide linker, a flexible linker, a rigid linker, a helical linker, and a non-helical linker. Any suitable linkers known in the art can be used. In certain embodiments, the linker comprises a peptide linker. For example, a useful linker in the present disclosure may be rich in glycine and serine residues. Examples include linkers having a single or repeated sequences comprising threonine/serine and glycine, such as TGGGG (SEQ ID NO: 24), GGGGS (SEQ ID NO: 25) or SGGGG (SEQ ID NO: 26) or its tandem repeats (e.g. 2, 3, 4, 5, 6, 7, 8, 9, 10 or more repeats). In certain embodiments, the linker used in the present disclosure comprises GGGGSGGGGSGGGGS (SEQ ID NO: 27). Alternatively, a linker may be a long peptide chain containing one or more sequential or tandem repeats of the amino acid sequence of GAPGGGGGAAAAAGGGGG (SEQ ID NO: 28). In certain embodiment, the linker comprises 1, 2, 3, 4, 5, 6, 7, 8, 9, 10 or more sequential or tandem repeats of SEQ ID NO: 28. In certain embodiments, the peptide linker comprises a GS linker. In certain embodiments, the GS linker comprises one or more repeats of GGGS (SEQ ID NO: 29) or SEQ ID NO: 25. In certain embodiments, the linker comprises or consists of an amino acid sequence having at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 24-29.

As used herein, the letter “n” followed by a number indicates a given amino acid residue position in the first target-binding domain provided herein. For example, the position “n1” indicates a first particular position in the first target-binding domain, and the position “n2” indicates a second particular position in the first target-binding domain. All positions relating to antibody sequences are based on the EU numbering system or the EU index, and therefore the position is also sometimes referred to herein as EU position. The position can be in the heavy chain portion of the first target-binding domain, or in the light chain portion of the first target-binding domain. For example, according to the context, the position n1 can be in the first CH1 region and the position n2 can be in the first CL region.

As used herein, the term “position pair” means a pair of EU positions in an antibody fragment, such as in a target-binding domain. A position pair is expressed herein using two numbers separated by a colon (:). For example, an n1: n2 position pair of 128:118 means that n1 is EU position 128 (e.g. in the first CH1 region), and n2 is EU position 118 (e.g. in the first CL region).

Herein, by configuring the first and the second amino acid residues at one of the EU position pairs 128:118 and 173:160 to form a covalent bond (e.g. a disulfide bond), the specificity for the cognate pairing of the heavy chain and the light chain can be improved. The amino acid residues at these EU position pairs in a native antibody do not involve in formation of natural covalent bonds such as disulfide bonds. Therefore, the polypeptide complexes provided herein can be useful in reducing mispairing of a first target-binding domain with a second target-binding domain which do not interfere with the covalent bond formation at such EU position pair.

As used herein, a “disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains under physiological conditions, thereby forming an interchain bridge or interchain bond to form an interchain (if both from different chains) or intrachain disulfide bond (if both from a same chain). With regard to an antibody, the term “interchain disulfide bond” refers to a disulfide bond formed between the sulfydryl groups of the cysteine residues in a disulfide bond-forming cysteine pair in an immunoglobulin hinge region or between a light chain and a heavy chain at their respective constant regions. In this present disclosure, unless otherwise indicated, the “disulfide bond” refers to a disulfide bond formed between the immunoglobulin heavy chain and light chain at their CH1 and CL regions respectively, which could be a native/naturally occurring disulfide bond (e.g. at EU position pair 220:214 (for IgG1) or 131:214 (for IgG2 and IgG4), or any of the disulfide bonds at EU position pairs 128:118 and/or 173:160.

According to some embodiments, the covalent bond formed between the first amino acid residue and the second amino acid residue is a disulfide bond, and further optionally, the disulfide bond is formed between two cysteine residues. In other words, in this embodiment of the polypeptide complex, the covalent bond formed between the first CH1 and the first CL regions in the first target-binding domain is a disulfide bond formed between two cysteine residues at the EU position pair 128:118 or 173:160. In one embodiment, one or both of the first and the second amino acid residues are introduced by substitution of the corresponding native or wildtype amino acid residues at the EU positions in one of the above two indicated position pairs in the first CH1 and the first CL regions with cysteine residues, respectively. For example, the first amino acid residue at EU position n1 and the second amino acid residue at EU position n2 are both cysteine residues. To be more specific, such substitutions can be either (i) L128C:F118C, or (ii) V173C:Q160C (or E160C). In some embodiments, the first CH1 region comprises a substitution of L128C (EU position n1) and the first CL region comprises a substation of F118C (EU position n2). It is noted, however, in addition to being an introduced disulfide bond, the covalent bond can also be of a different type of covalent bond. Examples as such can include an isopeptide bond formed between lysine and asparagine or aspartate, or a covalent bond formed between an unnatural amino acid (Uaa) and a cysteine residue (Nat Methods. 2013; 10(9): doi: 10.1038/nmeth.2595), and so on.

As used herein, the word “introduced,” “introduce,” or alike, is construed to refer to a state of an element or a structure that is absent in an original version of a larger entity or structure, and is brought about in a new version as an altered, modified or newly added element or structure therein.

The term “associated with,” “in association with,” or the like refers to, when with respect to two regions of a polypeptide complex (e.g. an antibody or an antigen-binding fragment thereof), the situation where the two regions are operably linked to each other, with or without a spacer or linker, in such a way that they are in a relationship permitting them to function in an intended manner. For example, a variable region of an antibody may be associated with a constant region so as to provide for a stable product with antigen-binding activity. The terms “associated with,” “in association with,” “linked to,” “coupled to,” “fused to,” “connected to,” “bound to” may be used interchangeably in the present disclosure.

According to some preferred embodiments of the polypeptide complex, the first target-binding domain is further modified (e.g. mutated) such that a natural disulfide bond in the first target-binding domain is disrupted. In one embodiment as such, the first target-binding domain comprises a substitution or deletion of cysteine residue at EU position 220 (for IgG1) or EU position 131 (for IgG2 and IgG4) of the heavy chain and/or comprises another substitution or deletion of cysteine residue at EU position 214 of the light chain.

As used in this disclosure, the polypeptide complex can be an antibody, or a fragment of an antibody that contains a target-binding domain. Examples of the polypeptide complex can include, without any limitation on the scope of the disclosure, a full-length antibody (e.g. monospecific antibody, bispecific antibody, triple-specific antibody, a bivalent antibody, a multiple valent antibody, etc.), an antigen-binding domain containing fragment thereof (e.g. Fab, Fab′, F(ab′)₂, etc.), or a protein complex comprising the antibody or its fragment. The polypeptide complex can also be any other type of molecule as long as such molecule contains a target-binding domain/region having the features as described above.

In some of these embodiments, the first CH1 region further comprises a third amino acid residue at EU position n3, and the first CL region further comprises a fourth amino acid residue at EU position n4, wherein n3:n4 position pair is selected from the group consisting of 183:176, 141:116, and 126:121; wherein the third amino acid residue and the fourth amino acid residue form a non-covalent bond.

It is unexpectedly found by the present inventors that, by combining the covalent bond between residues at the n1:n2 position pair and the non-covalent bond (such as electrostatic interaction) at the n3:n4 position pair, the polypeptide complexes provided herein are unexpectedly advantageous in reducing light chain mispairing and significantly improving the purity of the expressed products. Such a unique construct is easy to manufacture, yet providing much higher purity and yield than comparable constructs lacking such a combination.

As used herein, the term “non-covalent bond” refers to a non-covalent interaction between two peptide chains in a protein complex. Examples of a non-covalent bond include a hydrogen bond, electrostatic interaction, a salt bridge, or hydrophobic-hydrophilic interaction, a knobs-into-holes or a combination thereof. The non-covalent bonds and the covalent bonds (e.g. disulfide bonds) together forms a class of interchain bonds, which are beneficial for the folding, conformation, stability, flexibility, and functions of any protein, polypeptide, or a complex thereof.

In some embodiments, the non-covalent bond is an electrostatic interaction. In some embodiments, the third amino acid residue and the fourth amino acid residue are oppositely charged. Herein, by configuring that the third amino acid residue and the fourth amino acid residues at one or more of the four EU position pairs 183:176, 141:116, 126:121, and 218:122, are oppositely charged, it substantially creates one or more interactions between each pair of oppositely charged amino acid residues so as to form one or more non-covalent bonds therebetween, which in turn facilitates the specific cognate pairing of the heavy chain and light chain in the polypeptide complex disclosed herein.

As used herein, an “electrostatic interaction” is a non-covalent bond and commonly includes ionic interactions, hydrogen bonding and halogen bonding. Electrostatic interactions can be formed in a polypeptide or between different subunits/chains in a protein complex, for example, between Lys (K) and Asp (D), between Lys (K) and Glu (E), between Glu (E) and Arg (R), or between Glu (E), Trp (W) on the first chain and Arg (R), Val (V) or Thr (T) on the second chain.

A “salt bridge” is close-range electrostatic interactions that mainly arises from the anionic carboxylate of either Asp (D) or Glu (E) and the cationic ammonium from Lys (K) or the guanidinium of Arg (R), which are spatially proximal pairs of oppositely charged residues in a protein or polypeptide structures. Charged and polar residues in largely hydrophobic interfaces may act as hot spots for binding. Among others, residues with ionizable side chains such as His, Tyr, and Ser can also participate the formation of a salt bridge.

“Knobs-into-holes” as used herein, refers to an interaction between two polypeptides, where one polypeptide has a protuberance (i.e. “knob”) due to presence of an amino acid residue having a bulky side chain (e.g. tyrosine or tryptophan), and the other polypeptide has a cavity (i.e. “hole”) where a small side chain amino acid residue resides (e.g. alanine or threonine), and the protuberance is positionable in the cavity so as to promote interaction of the two polypeptides to form a heterodimer or a complex. Generally, the “knobs-into-holes” technique substantially introduces mutations in each of the two polypeptides in the Fc region to restrict a heavy chain-heavy chain combinations (Ridgway et al., Protein Engineering, 9(7), pp. 617-21(1996); Merchant et al., Nature Biotechnology, 16(7), pp. 677-681(1998)). Methods of generating polypeptides with knobs-into-holes are known in the art, e.g., as described in U.S. Pat. No. 5,731,168. It is noted that although the oppositely charged third and fourth amino acid residues at one or more of the three EU position pairs 183:176, 141:116, and 126:121 do not form Knobs-into-holes type of non-covalent interactions, it is possible that the first CH1 and the first CL regions contain other introduced residues (substitution, insertion, modification, etc.) that realize it.

As used herein, the term “oppositely charged” with respect to two amino acid residues means that both the two amino acid residues are charged, with one residue being positive-charged, while the other is negative-charged, at a physiological pH (e.g. at a pH value of approximately 7-7.5, preferably 7.4). For example, it could be that the first amino acid residue is positive-charged, while the second amino acid residue is negative-charged, or that the first amino acid residue is negative-charged, while the second amino acid residue is positive-charged. For clarification, two amino acid residues are not “oppositely charged” when: (a) one amino acid residue is charged, and the other amino acid residue is not charged; (b) both amino acid residues are not charged; (c) both amino acid residues are “like-charged”, i.e., one amino acid residue is positive-charged, and the other amino acid residue is also positive-charged; or one amino acid residue is negative-charged, and the other amino acid residue is also negative-charged.

As used herein, the term “positive-charged amino acid residue” or “positively charged amino acid residue” refers to an amino acid residue having a side chain that is positively charged under physiological condition (e.g. a pH of approximately 7-7.5, preferably 7.4). Although such positive-charged amino acid residues are typically natural amino acid residues such as lysine residue (K), arginine residue (R), and histidine residue (H), they can also include other amino acid analogs, mimetics, modifications that exhibit positive charges under physiological condition.

As used herein, the term “negative-charged amino acid residue” or “negatively charged amino acid residue” refers to an amino acid residue having a side chain that is negatively charged under physiological condition (e.g. a pH of approximately 7-7.5, preferably 7.4). Although such negative-charged amino acid residues are typically natural amino acid residues such as aspartic acid residue (D) and glutamic acid residue (E), they can also include other amino acid analogs, mimetics, modifications that exhibit negative charges under physiological condition.

In some embodiments, the polypeptide complex provided herein further comprises a second target-binding domain comprising a second target-binding moiety operably linked to a second constant moiety, wherein the second constant moiety comprises a second CH1 region associated with a second CL region, wherein the first CH1 region does not substantially bind to the second CL region, and the second CH1 region does not substantially bind to the first CL region.

The term “not substantially bind to” as used herein means that a given CH1 region and a mispairing CL region are significantly less prone to bind to each other and form a binding complex, in comparison with the given CH1 region and its pairing CL region. For example, the first CH1 region pairs with the first CL region, and does not substantially bind to the second CL region, and in such case, the first CH1 region and the second CL region are significantly less prone to bind to each other to form a binding complex, for example, the amount of binding complex between the first CH1 region and the second CL region would be much less (e.g. at least 30% less, at least 40% less, at least 50% less, at least 60% less, at least 70% less, at least 80% less) than the amount of binding complex between the first CH1 region and the first CL region. In some embodiments, element A does not substantially bind to element B means element A does not covalently bind to element B. For example, in some embodiments, the first CH1 region does not covalently bind to the second CL region, and the second CH1 region does not covalently bind to the first CL region.

In some embodiments, the second CH1 region comprises a first corresponding amino acid residue at EU position n1′, and the second CL region comprises a second corresponding amino acid residue at EU position n2′, wherein n1′:n2′ position pair is identical to the n1:n2 position pair, and wherein the first corresponding amino acid residue at EU position n1′ does not form a covalent bond with the second amino acid residue at EU position n2, and/or the second corresponding amino acid residue at EU position n2′ does not form a covalent bond with the first amino acid residue at EU position n1.

Without being bound to any theory, but it is believed that such a situation would avoid the cross-binding of the respective heavy chain portions and light chain portions forming the first and second target-binding domains, and thereby avoid or reduce their mispairings. Nevertheless, it shall be noted that, in the second target-binding domain, the first corresponding amino acid residue at EU position n1′ may form a covalent bond with the second corresponding amino acid residue at EU position n2′. For example, when the first amino acid residue at EU position n1 and the second amino acid residue at EU position n2 form a disulfide bond, the first corresponding amino acid residue at EU position n1′ and the second corresponding amino acid residue at EU position n2′ may form a covalent bond which is not a disulfide bond, the first and second corresponding amino acid residues that form the non-disulfide bond at EU position n1′ and n2′ do not bind to the first and second amino acid residues that form a disulfide bond at EU position n1 and n2, respectively.

As used herein, the letter “n” followed by a number with an apostrophe (′) indicates a given amino acid residue position in the second target-binding domain provided herein, which position corresponds to the given position in the first target-binding domain. For example, the position “n1′” indicates the position in the second target-binding domain that corresponds to the position n1 in the first target-binding domain, and the position “n2′” indicates the position in the second target-binding domain that corresponds to the position n2 in the first target-binding domain. In other words, when position n1 is determined, then position n1′ would also be determined, and vice versa. For example, if EU position n1 is EU position 128 in the first CH1 region in the first target-binding domain, then EU position n1′ would be EU position 128 in the second CH1 region in the second target-binding domain.

The amino acid residue in the second target-binding domain that corresponds to a counterpart in the first target-binding domain, is referred to herein as a “corresponding amino acid residue.” When a position n1 is determined, then position n1′ would be determined, and the residue at position n1 as well as the corresponding amino acid residue at position n1′ would both be determined.

In some embodiments, the first corresponding amino acid residue at EU position n1′ and the second corresponding amino acid residue at EU position n2′ do not form a covalent bond. In such embodiments, the CH1 region and CL region of the second target-binding domain do not associate with each other through a covalent bond at EU position n1′ and n2′. For example, the first and second corresponding amino acid residues at EU position n1′ and n2′ are native/wild-type amino acid residues, for example, selected from the amino acid pairs of L128:F118, V173:Q160 (or E160). For another example, the first and second corresponding amino acid residues at EU position n1′ and n2′ are mutated amino acid residues which do not form a covalent bond.

The term “mutation” or “mutated” with regard to an amino acid residue as used herein refers to substitution, replacement, insertion, addition, or modification of the amino acid residue. The term “substitution” or “substituted” with regard to an amino acid residue as used herein refers to the replacement of amino acid residue X (i.e. the amino acid residue before replacement, “X”) at position p with amino acid residue Z (i.e. the amino acid residue after replacement, “Z”) in a peptide, polypeptide or protein, and is denoted by XpZ. In one example, S183K denotes that an original native serine residue (S) at the EU position 183 of the CH1 region of the immunoglobulin heavy chain is substituted by a lysine residue (K).

In some embodiments, the second CH1 region further comprises a third corresponding amino acid residue at EU position n3′, and the second CL region further comprises a fourth corresponding amino acid residue at EU position n4′, wherein the n3′:n4′ position pair is identical to the n3:n4 position pair, and wherein the fourth corresponding amino acid residue at EU position n4′ and the third amino acid residue at EU position n3 are not oppositely charged or are like-charged.

In some embodiments, the second CH1 region further comprises a third corresponding amino acid residue at EU position n3′, and the second CL region further comprises a fourth corresponding amino acid residue at EU position n4′, wherein the n3′:n4′ position pair is identical to the n3:n4 position pair, and wherein the third corresponding amino acid residue at EU position n3′ and the fourth amino acid residue at EU position n4 are not oppositely charged or are like-charged.

Herein, the third and fourth corresponding amino acid residues at EU position n3′ and n4′ of the second target-binding domain do not interfere with the electrostatic interaction of the third and fourth amino acid residues at EU position n3 and n4 of the first target-binding domain.

In some embodiments, the third and fourth corresponding amino acid residues at EU position n3′ and n4′ are native/wild-type amino acid residues, for example, selected from the amino acid pairs of S183:S176, A141:F116, F126:S121, K218:D122. In some embodiments, one or both of the third and fourth corresponding amino acid residues at EU position n3′ and n4′ are mutated amino acid residues in the CH1 or CL region of the heavy or light chain of the second target-binding domain. For example, one or both of the third and fourth corresponding amino acid residues at EU position n3′ and n4′ are mutated amino acid residues which is(are) like-charged with the fourth and third amino acid residues at EU position n4 and n3, respectively, of the first target-binding domain.

In some embodiments, the third corresponding amino acid residue at EU position n3′ and/or the fourth corresponding amino acid residue at EU position n4′ are not charged. For example, the second target-binding domain does not have electrostatic interaction between the third corresponding amino acid residue at EU position n3′ and the fourth corresponding amino acid residue at EU position n4′.

In some embodiments, the second CH1 region further comprises a fifth corresponding amino acid residue at EU position n5′, and the second CL region further comprises a sixth corresponding amino acid residue at EU position n6′, and wherein the fifth corresponding amino acid residue and the sixth corresponding amino acid residue form a covalent bond, wherein n5′:n6′ position pair is different from the n1:n2 position pair.

Herein, the second CH1 region and the second CL region are also associated with each other through a covalent bond, but the positions of the amino acid residues forming the covalent bond are different from the positions of the amino acid residues forming the covalent bond in the first target-binding domain. For example, both the n5′:n6′ position pair and the n1:n2 position pair are independently selected from the group consisting of 220:214 (for IgG1), 131:214 (for IgG2 and IgG4), 128:118 and 173:160, provided that the n5′:n6′ position pair is different from the n1:n2 position pair. For example, when the n5′:n6′ position pair is 220:214 (for IgG1) or 131:214 (for IgG2 and IgG4), then the n1:n2 position pair is 128:118 or 173:160. For another example, the n5′:n6′ position pair is 128:118, and the n1:n2 position pair is 173:160. For yet another example, the n5′:n6′ position pair is 173:160, and the n1:n2 position pair is 128:118.

In some embodiments, the fifth and sixth corresponding amino acid residues at EU position n5′ and n6′ are native/wild-type amino acid residues, for example, selected from the amino acid pairs of C220:C214 (for IgG1), C131:C214 (for IgG2 and IgG4), L128:F118, V173:Q160 (or E160), provided that the n5′:n6′ position pair is different from the n1:n2 position pair. In some embodiments, the fifth corresponding amino acid residue at EU position n5′ and the sixth corresponding amino acid residue at EU position n6′ are both cysteine residues. For example, the second CH1 region comprises a substitution of L128C (EU position n5′) and the second CL region comprises a substation of F118C (EU position n6′). For another example, the second CH1 region comprises a substitution of V173C (EU position n5′) and the second CL region comprises a substation of Q160C (EU position n6′) for a kappa light chain or E160C (EU position n6′) for a lambda light chain.

In some embodiments, one or both of the fifth and sixth corresponding amino acid residues at EU position n5′ and n6′ are mutated amino acid residues in the CH1 or CL region of the heavy or light chain of the second target-binding domain. For example, one or both of the fifth and sixth corresponding amino acid residues at EU position n5′ and n6′ are mutated amino acid residues which form a covalent bond, provided that the n5′:n6′ position pair is different from the n1:n2 position pair.

As is known in the art, a native/wild-type disulfide bond is formed between the cysteine at EU position 220 (for IgG1), or 131 (for IgG2 and IgG4) of the heavy chain and the cysteine at EU position 214 of the light chain. In some embodiments, at least one of the first CH1 region and the second CH1 region has an amino acid residue other than cysteine at EU position 220 (for IgG1) or 131 (for IgG2 and IgG4), and/or at least one of the first CL region and the second CL region has an amino acid residue other than cysteine at EU position 214. By doing so, the native/wild-type disulfide bond in at least one of the first target-binding domain and the second target-binding domain is disrupted. For example, the first CH1 region has an amino acid residue other than cysteine at EU position 220 (for IgG1) or 131 (for IgG2 and IgG4) and the first CL region has an amino acid residue other than cysteine at EU position 214. For another example, the second CH1 region has an amino acid residue other than cysteine at EU position 220 (for IgG1) or 131 (for IgG2 and IgG4) and the second CL region has an amino acid residue other than cysteine at EU position 214.

Herein, by configuring an covalent bond (e.g. a disulfide bond) and one or more non-covalent bonds, and further preferably by simultaneously disrupting the natural disulfide bond, between the immunoglobulin light and heavy chains at their respective CL and CH1 regions, it maximizes the pairing accuracy and specificity for the light and heavy chains in the polypeptide complex, which in turn can further improve the generation of heterodimeric antibodies or antigen-binding fragment thereof having bispecific activity, which will be described in more detail in the following.

It is further noted that in the preferred embodiments as described above, by simultaneously disrupting the natural disulfide bond at EU position pair 220:214 (for IgG1) or 131:214 (for IgG2 and IgG4) in the same domain (i.e., the first target-binding domain), the potential interference from any natural cysteine residue at position 220 (for IgG1) or 131 (for IgG2 and IgG4) or 214 to any of the introduced cysteine residue can be reduced or eliminated, which can further improve the cognate pairing accuracy and specificity.

In some embodiments, neither the first CH1 region nor the second CH1 region has a cysteine residue at EU position 220 (for IgG1) or 131 (for IgG2 and IgG4), and/or neither the first CL region nor the second CL region has a cysteine residue at EU position 214. By doing so, the native/wild-type disulfide bonds in both the first target-binding domain and the second target-binding domain are disrupted.

In some embodiments, the first CH1 region further comprises a fifth amino acid residue at EU position n5, and the first CL region further comprises a sixth amino acid residue at EU position n6, and wherein n5:n6 position pair is identical to the n5′:n6′ position pair, and wherein the fifth corresponding amino acid residue at EU position n5′ does not form a covalent bond with the sixth amino acid residue at EU position n6, and/or the sixth corresponding amino acid residue at EU position n6′ does not form a covalent bond with the fifth amino acid residue at EU position n5. Without being bound to any theory, but it is believed that such a situation would avoid the cross-binding of the respective heavy chain portions and light chain portions forming the first and second target-binding domains, and thereby avoid or reduce their mispairings. Nevertheless, it shall be noted that, in the first target-binding domain, the fifth amino acid residue at EU position n5 may form a covalent bond with the sixth amino acid residue at EU position n6. For example, when the fifth corresponding amino acid residue at EU position n5′ and the sixth corresponding amino acid residue at EU position n6′ form a disulfide bond, the fifth amino acid residue at EU position n5 and the sixth amino acid residue at EU position n6 may form a covalent bond which is not a disulfide bond, the fifth and sixth amino acid residues that form the non-disulfide bond at EU position n5 and n6 do not bind to the fifth and sixth corresponding amino acid residues that form a disulfide bond at EU position n5′ and n6′, respectively.

In some embodiments, the fifth amino acid residue at EU position n5 and the sixth amino acid residue at EU position n6 do not form a covalent bond. In such embodiments, the CH1 region and CL region of the first target-binding domain do not associate with each other through a covalent bond at EU position n5 and n6. For example, the fifth and sixth amino acid residues at EU position n5 and n6 are native/wild-type amino acid residues, for example, selected from the amino acid pairs of L128:F118, V173:Q160 (or E160). For another example, the fifth and sixth amino acid residues at EU position n5 and n6 are mutated amino acid residues which do not form a covalent bond.

In some embodiments, the second CH1 region further comprises a seventh corresponding amino acid residue at EU position n7′ and the second CL region further comprises an eighth corresponding amino acid residue at EU position n8′, wherein n7′:n8′ position pair is selected from the group consisting of 183:176, 141:116, 126:121, and 218:122; wherein the seventh corresponding amino acid residue and the eighth corresponding amino acid residue are oppositely charged, and wherein the n7′:n8′ position pair is different from the n3:n4 position pair.

Herein, the second CH1 region and the second CL region are also associated with each other through an electrostatic interaction, but the positions of the amino acid residues forming the electrostatic interaction are different from the positions of the amino acid residues forming the electrostatic interaction in the first target-binding domain. For example, both the n7′:n8′ position pair and the n3:n4 position pare are independently selected from the group consisting of 183:176, 141:116, 126:121, and 218:122, provided that the n7′:n8′ position pair is different from the n3:n4 position pair. In some embodiments, both the n7′:n8′ position pair and the n3:n4 position pair are independently selected from the group consisting of 183:176, 141:116, and 126:121, provided that the n7′:n8′ position pair is different from the n3:n4 position pair. In some embodiments, the n7′:n8′ position pair is selected from the group consisting of 183:176, 141:116, and 126:121. For example, the n7′:n8′ position pair is 183:176, and the n3:n4 position pair is selected from the group consisting of 141:116, 126:121, and 218:122. For another example, the n7′:n8′ position pair is 141:116, and the n3:n4 position pair is selected from the group consisting of 183:176, 126:121, and 218:122. For yet another example, the n7′:n8′ position pair is 126:121, and the n3:n4 position pair is selected from the group consisting of 183:176, 141:116, and 218:122. For yet another example, the n7′:n8′ position pair is 218:122, and the n3:n4 position pair is selected from the group consisting of 183:176, 141:116, and 126:121.

In some embodiments, the seventh and eighth corresponding amino acid residues at EU position n7′ and n8′ are native/wild-type amino acid residues, for example, selected from the amino acid pairs of S183:S176, A141:F116, F126:S121, and K218:D122, provided that the n7′:n8′ position pair is different from the n3:n4 position pair. In some embodiments, one or both of the seventh and eighth corresponding amino acid residues at EU position n7′ and n8′ are mutated amino acid residues in the CH1 or CL region of the heavy or light chain of the second target-binding domain. For example, one or both of the seventh and eighth corresponding amino acid residues at EU position n7′ and n8′ are mutated amino acid residues which are oppositely charged, provided that the n7′:n8′ position pair is different from the n3:n4 position pair.

In some embodiments, the first CH1 region further comprises a seventh amino acid residue at EU position n7, and the second CL region further comprises an eighth amino acid residue at EU position n8, wherein the n7:n8 position pair is identical to the n7′:n8′ position pair, and wherein the seventh corresponding amino acid residue at EU position n7′ and the eighth amino acid residue at EU position n8 are not oppositely charged or are like-charged, and/or the eighth corresponding amino acid residue at EU position n8′ and the seventh amino acid residue at EU position n7 are not oppositely charged or are like-charged.

Herein, the seventh and eighth amino acid residues at EU position n7 and n8 of the first target-binding domain do not interfere with the electrostatic interaction of the seventh and eighth corresponding amino acid residues at EU position n7′ and n8′ of the second target-binding domain.

In some embodiments, the seventh and eighth amino acid residues at EU position n7 and n8 are native/wild-type amino acid residues, for example, selected from the amino acid pairs of S183:S176, A141:F1116, F126:S121, and K218:D122. In some embodiments, one or both of the seventh and eighth amino acid residues at EU position n7 and n8 are mutated amino acid residues in the CH1 or CL region of the heavy or light chain of the second target-binding domain. For example, one or both of the seventh and eighth amino acid residues at EU position n7 and n8 are mutated amino acid residues which is(are) like-charged with the eighth and seventh corresponding amino acid residues at EU position n8′ and n7′ of the second target-binding domain.

In some embodiments, the seventh amino acid residue at EU position n7 and/or the eighth amino acid residue at EU position n8 are not charged. For example, the first target-binding domain does not have electrostatic interaction between the seventh amino acid residue at EU position n7 and the eighth amino acid residue at EU position n8.

In some embodiments, at least one, two, three, or four of the first amino acid residue at EU position n1, the second amino acid residue at EU position n2, the third amino acid residue at EU position n3, and fourth amino acid residue at EU position n4 are introduced by substitution.

Herein, each of the first, second, third, and fourth introduced amino acid residues can be an amino acid residue that is absent in a native/wildtype version of, and is thus “introduced” at any of the above indicated EU positions (i.e. 128:118 and 173:160 for the first and second introduced amino acid residues, and 183:176, 141:116, 126:121, and 218:122 for the third and fourth introduced amino acid residues) in, the first CH1 or the first CL region of an immunoglobulin. Any such an introduced amino acid residue may be a substituted amino acid that replaces a wildtype residue, or may be a newly added/inserted residue that is absent in a wildtype polypeptide, or may be a modified residue or an artificial residue.

In some other embodiments, at least one, two, three, or four of the first amino acid residue at EU position n1, the second amino acid residue at EU position n2, the third amino acid residue at EU position n3, and fourth amino acid residue at EU position n4 can be amino acid residue(s) that are newly inserted/added at the EU position n1, n2, n3 and/or n4 without substitution(s).

In yet some other embodiments, one or both of the third and the fourth amino acid residues at EU position n3 and n4 can be a non-natural amino acid analog(s) or mimic(s), or a chemically modified amino acid residue(s) which is/are positively or negatively charged.

In certain embodiments, there may exist more than one pair of oppositely charged third and fourth amino acid residues at the EU position pairs 183:176, 141:116, 126:121, and 218:122. For one example, the polypeptide complex may be configured such that each of the two amino acid residue pairs at both EU position pairs 183: 176 and 141:116 is mutated (e.g. substituted, inserted, or modified) to be oppositely charged. For another example, all of the four amino acid residue pairs at EU position pairs 183:176, 141:116, 126:121, and 218:122 are mutated (e.g. substituted, inserted, or modified) to be oppositely charged.

As such, for each n3:n4 pair selected from EU position pairs 183:176, 141:116, 126:121, and 218:122, two alternative schemes can be applied. In a first scheme, the third amino acid residue at EU position n3 is a positive-charged amino acid residue, such as lysine (K), arginine (R) or histidine (H), and the fourth amino acid residue at EU position n4 is a negative-charged amino acid residue, such as aspartic acid (D) or glutamic acid (E). In a second scheme, the third amino acid residue at EU position n3 is a negative-charged amino acid residue, such as aspartic acid (D) or glutamic acid (E), and the fourth amino acid residue at EU position n4 is a positive-charged amino acid residue, such as lysine (K), arginine (R) or histidine (H).

To summarize all of the combinations in the above two schemes, the third amino acid residue and the fourth amino acid residue at the n3:n4 position pair are substitutions selected from the group consisting of: S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R.

Similarly, in some embodiments, the seventh corresponding amino acid residue at EU position n7′ is a positive-charged amino acid residue, and the eighth corresponding amino acid residue at EU position n8′ is a negative-charged amino acid residue. In some embodiments, the seventh corresponding amino acid residue at EU position n7′ is a negative-charged amino acid residue, and the eighth corresponding amino acid residue at EU position n8′ is a positive-charged amino acid residue.

In some embodiments, at least one, two, three, or four of the fifth corresponding amino acid residue at EU position n5′, the sixth corresponding amino acid residue at EU position n6′, the seventh corresponding amino acid residue at EU position n7′, and the eighth corresponding amino acid residue at EU position n8′ are introduced by substitution.

In some other embodiments, at least one, two, three, or four of the fifth corresponding amino acid residue at EU position n5′, the sixth corresponding amino acid residue at EU position n6′, the seventh corresponding amino acid residue at EU position n7′, and the eighth corresponding amino acid residue at EU position n8′ can be amino acid residue(s) that are newly inserted/added at the EU position n5′, n6′, n7′ and/or n8′ without substitution(s).

In yet some other embodiments, one or both of the seventh and the eighth corresponding amino acid residues at EU position n7′ and n8′ can be a non-natural amino acid analog(s) or mimic(s), or a chemically modified amino acid residue(s) which is/are positively or negatively charged.

In certain embodiments, there may exist more than one pair of oppositely charged seventh and eighth corresponding amino acid residues at the EU position pairs 183:176, 141:116, 126:121, and 218:122. For one example, the polypeptide complex may be configured such that each of the two amino acid residue pairs at both EU position pairs 183: 176 and 141:116 is mutated (e.g. substituted, inserted, or modified) to be oppositely charged. For another example, all of the four amino acid residue pairs at EU position pairs 183:176, 141:116, 126:121, and 218:122 are mutated (e.g. substituted, inserted, or modified) to be oppositely charged.

As such, for each n7′:n8′ pair selected from EU position pairs 183:176, 141:116, and 126:121, two alternative schemes can be applied. In a first scheme, the seventh corresponding amino acid residue at EU position n7′ is a positive-charged amino acid residue, such as lysine (K), arginine (R) or histidine (H), and the eighth corresponding amino acid residue at EU position n8′ is a negative-charged amino acid residue, such as aspartic acid (D) or glutamic acid (E). In a second scheme, the seventh corresponding amino acid residue at EU position n7′ is a negative-charged amino acid residue, such as aspartic acid (D) or glutamic acid (E), and the eighth corresponding amino acid residue at EU position n8′ is a positive-charged amino acid residue, such as lysine (K), arginine (R) or histidine (H).

To summarize all of the combinations in the above two schemes, the seventh corresponding amino acid residue and the eighth corresponding amino acid residue at the n7′:n8′ position pair are substitutions selected from the group consisting of S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R, and wherein the n7′:n8′ position pair is different from the n3:n4 position pair.

In some embodiments, the first target-binding domain comprises a first combination of substitutions at (n1+n2):(n3+n4) positions, and/or the second target-binding domain comprises a second combination of substitutions at (n5′+n6′):(n7′+n8′) positions, and wherein the first combination of substitutions and/or the second combination of substitutions are selected from the group consisting of: (L128C+S183K):(F118C+S176D), (L128C+S183K):(F118C+S176E), (L128C+S183R):(F118C+S176D), (L128C+S183R):(F118C+S176E), (L128C+S183H):(F118C+S176D), (L128C+S183H):(F118C+S176E), (L128C+S183D):(F118C+S176K), (L128C+S183D):(F118C+S176R), (L128C+S183D):(F118C+S176H), (L128C+S183E):(F118C+S176K), (L128C+S183E):(F118C+S176R), (L128C+S183E):(F118C+S176H), (V173C+A141K):(Q160C (or E160C)+F116D), (V173C+A141K):(Q160C (or E160C)+F116E), (V173C+A141R):(Q160C (or E160C)+F116D), (V173C+A141R):(Q160C (or E160C)+F116E), (V173C+A141H):(Q160C (or E160C)+F116D), (V173C+A141H):(Q160C (or E160C)+F116E), (V173C+A141D):(Q160C (or E160C)+F116K), (V173C+A141D):(Q160C (or E160C)+F116R), (V173C+A141D):(Q160C (or E160C)+F116H), (V173C+A141E):(Q160C (or E160C)+F116K), (V173C+A141E):(Q160C (or E160C)+F116R), (V173C+A141E):(Q160C (or E160C)+F116H), (V173C+S183K):(Q160C (or E160C)+S176D), (V173C+S183K):(Q160C (or E160C)+S176E), (V173C+S183R):(Q160C (or E160C)+S176D), (V173C+S183R):(Q160C (or E160C)+S176E), (V173C+S183H):(Q160C (or E160C)+S176D), (V173C+S183H):(Q160C (or E160C)+S176E), (V173C+S183D):(Q160C (or E160C)+S176K), (V173C+S183D):(Q160C (or E160C)+S176R), (V173C+S183D):(Q160C (or E160C)+S176H), (V173C+S183E):(Q160C (or E160C)+S176K), (V173C+S183E):(Q160C (or E160C)+S176R), (V173C+S183E):(Q160C (or E160C)+S176H), (L128C+F126K):(F118C+S121D), (L128C+F126K):(F118C+S121E), (L128C+F126R):(F118C+S121D), (L128C+F126R):(F118C+S121E), (L128C+F126H):(F118C+S121D), (L128C+F126H):(F118C+S121E), (L128C+F126D):(F118C+S121K), (L128C+F126D):(F118C+S121R), (L128C+F126D):(F118C+S121H), (L128C+F126E):(F118C+S121K), (L128C+F126E):(F118C+S121R), (L128C+F126E):(F118C+S121H), (V173C+F126K):(Q160C (or E160C)+S121D), (V173C+F126K):(Q160C (or E160C)+S121E), (V173C+F126R):(Q160C (or E160C)+S121D), (V173C+F126R):(Q160C (or E160C)+S121E), (V173C+F126H):(Q160C (or E160C)+S121D), (V173C+F126H):(Q160C (or E160C)+S121E), (V173C+F126D):(Q160C (or E160C)+S121K), (V173C+F126D):(Q160C (or E160C)+S121R), (V173C+F126D):(Q160C (or E160C)+S121H), (V173C+F126E):(Q160C (or E160C)+S121K), (V173C+F126E):(Q160C (or E160C)+S121R), and (V173C+F126E):(Q160C (or E160C)+S121H), (L128C+K218D):(F118C+D122K), (L128C+K218D):(F118C+D122H), (L128C+K218D):(F118C+D122R), (L128C+K218E):(F118C+D122K), (L128C+K218E):(F118C+D122H), (L128C+K218E):(F118C+D122R), (V173C+K218D):(Q160C (or E160C)+D122K), (V173C+K218D):(Q160C (or E160C)+D122H), (V173C+K218D):(Q160C (or E160C)+D122R), (V173C+K218E):(Q160C (or E160C)+D122K), (V173C+K218E):(Q160C (or E160C)+D122H), and (V173C+K218E):(Q160C (or E160C)+D122R), provided that, when both the first combination of substitutions and the second combination of substitutions are selected, the n5′:n6′ position pair is different from the n1:n2 position pair, and the n7′:n8′ position pair is different from the n3:n4 position pair.

In some embodiments, the first or second target-binding domain comprises a substitution of L128C in the first or second CH1 region, and comprises a substitution of F118C in the first or second CL region.

In some embodiments, the first or second target-binding domain comprises a substitution of V173C in the first or second CH1 region, and comprises a substitution of Q160C (or E160C) in the first or second CL region.

In some embodiments, the first or second target-binding domain comprises substitutions of L128C and S183D in the first or second CH1 region, and comprises substitutions of F118C and S176K in the first or second CL region.

In some embodiments, the first or second target-binding domain comprises substitutions of L128C and F126D in the first or second CH1 region, and comprises substitutions of F118C and S121K in the first or second CL region.

In some embodiments, the first or second target-binding domain comprises substitutions of L128C and A141D in the first or second CH1 region, and comprises substitutions of F118C and F116K in the first or second CL region.

In some embodiments, the first or second target-binding domain comprises substitutions of L128C and K218D in the first or second CH1 region, and comprises substitutions of F118C and D122K in the first or second CL region.

In some embodiments, the first or second target-binding domain comprises substitutions of V173C and S183D in the first or second CH1 region, and comprises substitutions of Q160C and S176K in the first or second CL region.

In some embodiments, the first or second target-binding domain comprises substitutions of V173C and A141D in the first or second CH1 region, and comprises substitutions of Q160C and F116K in the first or second CL region.

In some embodiments, the first or second target-binding domain comprises substitutions of V173C and S183D in the first or second CH1 region.

In some embodiments, the first or second target-binding domain comprises substitutions of V173C and S183D in the first or second CH1 region, and comprises Q160C (or E160C) and S176K in the first or second CL region.

In some embodiments, the first or second target-binding domain comprises substitutions of L128C and C220S (for IgG1) or C131S (for IgG2 and IgG4) in the first or second CH1 region, and comprises substitutions of F118C and C214S in the first or second CL region.

In some embodiments, the first or second target-binding domain comprises substitutions of V173C and C220S (for IgG1) or C131S (for IgG2 and IgG4) in the first or second CH1 region, and comprises substitutions of Q160C (or E160C) and C214S in the first or second CL region.

In some embodiments, the first or second target-binding domain comprises substitutions of L128C, C220S for IgG1 (or C131S for IgG2 and IgG4) and S183D in the first or second CH1 region, and comprises substitutions of F118C, C214S and S176K in the first or second CL region.

In some embodiments, the first or second target-binding domain comprises substitutions of L128C, C220S for IgG1 (or C131S for IgG2 and IgG4) and F126D in the first or second CH1 region, and comprises substitutions of F118C, C214S and S121K in the first or second CL region.

In some embodiments, the first or second target-binding domain comprises substitutions of L128C, C220S for IgG1 (or C131S for IgG2 and IgG4) and A141D in the first or second CH1 region, and comprises substitutions of F118C, C214S and F116K in the first or second CL region.

In some embodiments, the first or second target-binding domain comprises substitutions of L128C, C220S for IgG1 (or C131S for IgG2 and IgG4) and K218D in the first or second CH1 region, and comprises substitutions of F118C, C214S and D122K in the first or second CL region.

In some embodiments, the first or second target-binding domain comprises substitutions of V173C, C220S for IgG1 (or C131S for IgG2 and IgG4) and S183D in the first or second CH1 region, and comprises substitutions of Q160C, C214S and S176K in the first or second CL region.

In some embodiments, the first or second target-binding domain comprises substitutions of V173C, C220S for IgG1 (or C131S for IgG2 and IgG4) and A141D in the first or second CH1 region, and comprises substitutions of Q160C, C214S and F116K in the first or second CL region.

In some embodiments, the first or second target-binding domain comprises substitutions of V173C, C220S for IgG1 (or C131S for IgG2 and IgG4) and S183D in the first or second CH1 region.

In some embodiments, the first or second target-binding domain comprises substitutions of V173C, C220S for IgG1 (or C131S for IgG2 and IgG4) and S183D in the first or second CH1 region, and comprises Q160C (or E160C), C214S and S176K in the first or second CL region.

In some embodiments, the first target-binding moiety provided herein comprises a first polypeptide fragment operably linked to the first CL region, and the second target-binding moiety provided herein comprises a second polypeptide fragment operably linked to the second CL region, wherein the first polypeptide fragment has a different amino acid sequence from the second polypeptide fragment. In some embodiments, either the first polypeptide fragment or the second polypeptide fragment is absent from the polypeptide complex.

As used herein, “a different amino acid sequence” refers to one amino acid sequence is different from the other amino acid sequence in, for example, length, amino acid type, or function.

In some embodiments, the polypeptide complex can be a fusion protein that comprises two or more polypeptide fragments operably linked to a CH1 region and a CL region, respectively. In some embodiments, the first target-binding moiety further comprises a third polypeptide fragment operably linked to the first CH1 region, and the second target-binding moiety comprises a fourth polypeptide fragment operably linked to the second CH1 region. In some embodiments, the third polypeptide fragment has a different amino acid sequence from the fourth polypeptide fragment. In some embodiments, either the third polypeptide fragment or the fourth polypeptide fragment is absent from the polypeptide complex.

In some embodiments, the first polypeptide fragment and the third polypeptide fragment can each contain a target-binding site and bind to its target molecule. For example, the first polypeptide fragment and the third polypeptide fragment can bind to the same target molecule, or alternatively binds to different target molecules. For another example, the first polypeptide fragment and the third polypeptide fragment can have either identical or different amino acid sequences.

Likewise, the second polypeptide fragment and the fourth polypeptide fragment can each contain a target-binding site and bind to its target molecule. For example, the second polypeptide fragment and the fourth polypeptide fragment can bind to the same target molecule, or alternatively bind to different target molecules. For another example, the second polypeptide fragment and the fourth polypeptide fragment can have either identical or different amino acid sequences.

In some embodiments, the first polypeptide fragment and the third polypeptide fragment can be associated to form a first target-binding site. Likewise, the second polypeptide fragment and the fourth polypeptide fragment can be associated to form a second target-binding site. In some embodiments, the first target-binding site and the second target-binding site can bind to the same target molecule, or different parts on the same target molecule, or different target molecules.

In some embodiments, the first target-binding moiety can be a first antigen-binding moiety and/or the second target-binding moiety can be a second antigen-binding moiety. In some embodiments, the antigen-binding moiety is derived from one or more antibody fragments.

In some embodiments, the first antigen-binding moiety can comprise a first VL region and a first VH region, which are associated to form a first antigen-binding site. In some embodiments, the second antigen-binding moiety can comprise a second VL region and a second VH region, which are associated to form a second antigen-binding site. The first antigen-binding site and the second antigen-binding site may bind to the same antigen, or different epitopes on the same antigen, or different antigens.

In some embodiments, the first and/or the second antigen-binding domain provided herein is selected from the group consisting of a Fab domain, a Fab′, and a F(ab′)₂, the first antigen-binding domain and/or the second antigen-binding domain comprises one or more CDRs operably linked to a CH1 region and a CL region. In some embodiments, the first antigen-binding domain comprises a first Fab domain. In some embodiments, the second antigen-binding domain comprises a second Fab domain. In some embodiments, the second Fab domain comprises one or more light chain CDRs and/or light chain framework regions different from that of the first Fab domain. In some embodiments, the second Fab domain further comprises one or more heavy chain CDRs and/or heavy chain framework regions different from that of the first Fab domain.

In some embodiments, the first Fab domain and/or the second Fab domain provided herein also encompass various variants of the Fab domain. For example, the variants of the Fab domain may comprise one or more modifications or substitutions in one or more of the amino acid residues of the CH1 region and/or CL region. Such variants may have one or more desirable properties conferred by the modification(s) or substitution(s), e.g. improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g. one or more introduced cysteine residues). In some embodiments, the Fab domain provided herein does not comprise a HCVR or LCVR. In some embodiments, the Fab domain provided herein comprises a HCVR, LCVR, CH1 region and CL region.

In some embodiments, the first Fab domain and the second Fab domain disclosed herein are two different Fab domains, which means the two Fab domains have one or more differences respectively regarding the primary sequences, side chain modifications or conformations of both the light chain portion and heavy chain portion constituting the Fab domains.

### III. Heterodimeric Antibodies or Antigen-Binding Fragment Thereof

In some embodiments, the first antigen-binding domain and/or the second antigen-binding domain is contained within an antibody, optionally a bispecific antibody or a multispecific antibody.

In some embodiments, the present disclosure provides heterodimeric protein complexes comprising the first antigen-binding domain and the second antigen-binding domain provided herein. In some embodiments, the heterodimeric protein complex provided herein comprises a heterodimeric antibody.

As used herein, the term “heterodimeric antibody” refers to an asymmetrical antibody having two different subunits associated with one another, which is in contrast with a native or naturally occurring antibody that is essentially a homodimer. Such a homodimer consists of two identical light chains and two identical heavy chains, conformationally grouped into a first pair of light chain and heavy chain and an identical second pair of light chain and heavy chain which constitute the two identical subunits, each containing an identical antigen-binding domain as each of the two arms of the Y-shaped antibody. In the context of this disclosure, the heterodimeric antibody at least comprises two different antigen-binding regions/domains (i.e. first and second antigen-binding domain), each comprising a constant moiety operably linked to a variable domain. Each constant moiety includes an immunoglobulin heavy chain constant region 1 (CH1) and an immunoglobulin light chain constant region (CL). Each antigen-binding domain contains a different antigen-binding moiety that binds specifically to a different epitope, and typically includes an immunoglobulin heavy chain variable region (VH) and an immunoglobulin light chain variable region (VL). The VH region and the CH1 region are operably linked to each other, and the VL region and the CL region are operably linked to each other. In each antigen-binding domain, the CL region and the CH1 region are associated with each other, by means of at least one non-native covalent interchain bond (e.g. disulfide bond) and at least one non-native non-covalent bond (e.g. electrostatic interaction or salt bridge). Further in the context of this disclosure, the heterodimeric antibody may optionally further comprise an Fc domain that is operatively linked to the first and the second antigen-binding domains. The Fc domain includes a first Fc polypeptide and a second Fc polypeptide, each comprising, for example, CH2 and CH3 regions of an immunoglobulin heavy chain. The Fc domain may be engineered (i.e., mutated or modified) to facilitate heterodimerization. Yet it is possible that the two different antigen-binding domains are associated with each other by means other than the Fc domain, for example, through a polypeptide, a covalent chemical bond, etc.

The heterodimeric antibody as used herein is essentially an engineered monoclonal antibody. As used herein, the term “monoclonal antibody” refers to a type of antibodies that are made by identical immune cells which are all clones belonging to a unique parent cell. Monoclonal antibodies are highly specific, being directed against a single epitope. Herein, the modifier “monoclonal” shall not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991).

The heterodimeric antibody or antigen-binding fragment thereof comprises a first antigen-binding domain and a second antigen-binding domain. The first antigen-binding domain and the second antigen-binding domain are two different domains. This configuration is favorable to the formation of the heterodimeric antibody or antigen-binding fragment thereof, because the first CH1 region selectively associates with the first CL region to form the first antigen-binding domain, with no substantial bonding between the first CH1 region with the second CL region or between the first CL region with the second CH1 region. This in turns allows for the efficient formation of the heterodimeric antibody or antigen-binding fragment thereof having two different antigen-binding domains, each potentially having a different antigen-binding moiety.

The first antigen-binding domain and the second antigen-binding domain described under Section II. Polypeptide complexes above can be also applied to the heterodimeric antibodies described herein.

In certain embodiments, the second antigen-binding domain comprises one or more light chain CDRs and/or light chain framework regions that are different from that of the first antigen-binding domain. In certain embodiments, the second antigen-binding domain comprises one or more heavy chain CDRs and/or heavy chain framework regions different from that of the first antigen-binding domain. By such a configuration, the first antigen-binding domain and the second antigen-binding domain are configured to specifically target and bind to different antigens or different epitopes of a same antigen.

In some embodiments, the heterodimeric antibody is a bispecific antibody. Herein, the heterodimeric antibody may have its two antigen-binding domains specifically targeting two different antigens or two different epitopes of a single antigen, respectively.

In some other embodiments, the heterodimeric antibody is a multispecific antibody. Herein, in addition to the two antigen-binding domains specifically targeting two different antigens or two different epitopes of a single antigen respectively, the heterodimeric antibody can further include one or more other antigen-binding moieties that specifically target other antigens or other epitopes. In one non-limiting example, an antigen-binding site can be engineered to be introduced into an Fc region of an originally bispecific antibody, which is called Fcab, to thereby acquire one additional antigen-binding site (Wozniak-Knopp G, et al., (2010). Protein Eng Des. 23 (4): 289-297.), and the antibodies thus obtained is a multispecific antibody that has three antigen-binding sites. Other examples may also exist.

In some embodiments, the second antigen-binding domain and the first antigen-binding domain bind to different antigens or alternatively bind to different epitopes on the same antigen.

In some embodiments, the antigen can be a tumor-associated antigen, immune related target, or an infectious agent related target.

In some embodiments, one of the first antigen-binding domain and the second antigen-binding domain binds to a tumor-associated antigen, and the other binds to an immune related target. In some embodiments, one of the first antigen-binding domain and the second antigen-binding domain binds to a first tumor-associated antigen, and the other binds to a second tumor-associated antigen. For detailed description about the tumor-associated antigen, please refer to Section VIII. Treatment. The immune related target provided herein may be selected from the group consisting of CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), BTLA (CD272), BTLA, CD160, CTLA-4 (CD152), IDO1, ID02, TDO, KIR, LAIR-1, NOX2, PD-1, PD-L1, PD-L2, TIM-3, VISTA, SIGLEC-7 (CD328), TIGIT, PVR (CD155), TGF 0, SIGLEC9 (CD329), and any combination thereof.

In some embodiments, each of the first CL region or the second CL region is derived from a kappa light chain. In some embodiments, each of the first CL region or the second CL region is derived from a lambda light chain. In some embodiments, the first CL region or the second CL region are derived from a kappa light chain and a lambda light chain, respectively.

According to different embodiments of the antibody or its antigen-binding fragment, the first and/or the second antigen-binding domain can be chimeric, humanized, or fully human.

As used herein, the term “chimeric” refer to a situation where a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

As used herein, the term “humanized” refers to a situation wherein a sequence of a polypeptide (e.g. antibody) from a non-human species is modified to increase their similarity to antibody variants produced naturally in humans, which is a specialized form of a chimeric polypeptide. For example, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Nat. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation). The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

In certain embodiments, the antibody or antigen-binding fragment thereof further comprises an Fc region/domain that is operably linked to the first and the second antigen-binding domains, which may increase the stability of the antibody or its antigen-binding fragment, but may also mediate the various effector functions such as antibody-dependent cellular cytotoxicity (ADCC) and complement-dependent cytotoxicity (CDC), etc., through the interaction with cell surface receptors (i.e. Fc receptors) and certain proteins in the complement system.

In certain embodiments, the Fc region is operably linked to both the first antigen-binding domain and the second antigen-binding domain through a spacer/linker. Optionally and preferably, such spacer is the natural hinge region existing in the two heavy chains of an immunoglobulin that joins their respective CH1 regions with their CH2 regions, and contains one or more interchain disulfide bonds forming interchain bridges connecting the two heavy chains. Yet alternatively, the spacer may be an artificial polypeptide liner that includes around 10-40 amino acid residues that is flexible to allow the Fc region to bind with both the first and the second antigen-binding domains without interfering with their respective functionalities. Further alternatively, the spacer may only represent a chemical bridge.

Optionally the Fc region is derived from IgG, IgA, IgM, IgE or IgD, and is preferably derived from IgG1, IgG2, IgG3 or IgG4, and is more preferably derived from IgG1.

Herein, the Fc region can be a wild type Fc region or a variant Fc region.

In certain preferred embodiments, the Fc region is a variant Fc region that is heterodimeric, comprising a first Fc polypeptide and a second Fc polypeptide. The variant Fc region further comprises one or more mutations that facilitate heterodimerization.

Engineering of multiple amino acid residues in the two immunoglobulin heavy chains at an interface therebetween in the Fc region, most notably their CH3 regions, have been shown to be able to improve the percentage of heterodimers recovered from recombinant cell culture. One strategy thus adopted is sometimes called a “knobs-into-holes” strategy, where one or more protrusions (i.e. “knobs”) can be generated by substituting one or more amino acid residues with small side chains (e.g. glycine, alanine, threonine) on the interface of the first heavy chain with amino acid residues with large side chains (e.g. tyrosine or tryptophan), and compensatory “cavities” (i.e. “holes”) with similar sizes to the large side chain(s) are simultaneously created on the interface of the second heavy chain by substituting amino acid residues with large side chains with amino acid residues with small side chains (e.g. alanine or threonine). In another strategy, the CH3 region may be modified to include mutations that introduce cysteine residues capable of forming a disulfide bond. These modifications provide a mechanism for increasing the yield of the heterodimer over other unwanted end-products such as homodimers. CH3 modifications to enhance heterodimerization include, for example, Y407V/T366S/L368A on one heavy chain and T366W on the other heavy chain; S354C, T366W on one heavy chain and Y349C/Y407V/T366S/L368A on the other heavy chain. Additional modifications resulting in a protrusion on one chain and a cavity on the other are described in U.S. Pat. Nos. 7,183,076 and 9,527,927; and Merchant et al., 1998, Nat. Biotech 16:677-681. Yet another strategy for engineering amino acid residues so as to facilitate the formation of heterodimers includes altering the charge polarity across the Fe dimer interface such that co-expression of electrostatically matched Fc regions results in heterodimerization. One such charge-pair mutations include T366K+L351D and L351K+Y349E/Y349D/L368E (WO 2013/157953), and other such charge-pair mutations are described in WO 2007/147901, WO 2012/058768, U.S. Pat. No. 9,527,927, WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, and Gunasekaran et al., 2010, JBC 285:19637-46. Yet another strategy that facilitates heterodimeric Fc formation is described in WO 2007/110205 and Davis et al. (2010), Prot. Eng. Design & Selection 23:195-202, which uses strand-exchanged engineered domain (SEED) CH3 regions which are derivatives of human IgG and IgA CH3 domains.

Herein optionally, the variant Fc region comprises a first Fc mutation in the first Fc polypeptide and/or a second Fc mutation in the second Fc polypeptide.

Further optionally, the first Fc mutation and the second Fc mutation are selected from any one of the following combinations:
a) the first Fc mutation comprises T366W or S354C, and the second Fc mutation comprises Y349C, T366S, L368A, or Y407V;
b) the first Fc mutation comprises D399K or E356K, and the second Fc mutation comprises K392D, or K409D;
c) the first Fc mutation comprises E356K, E357K, or D399K, and the second Fc mutation comprises K370E, K409D, or K439E;
d) the first Fc mutation comprises S364H, or F405A, and the second Fc mutation comprises Y349T, or T394F;
e) the first Fc mutation comprises S364H, or T394F, and the second Fc mutation comprises Y394T, or F405A;
f) the first Fc mutation comprises K370D, or K409D, and the second Fc mutation comprises E357K, or D399K; or
g) the first Fc mutation comprises L351D, or L368E, and the second Fc mutation comprises L351K, or T366K;

In any of the above, the numbering is according to the EU index.

### IV. Antibody Conjugates

The polypeptide complex as provided herein can be used in a non-conjugated form or in a conjugated form.

In a conjugated form, the polypeptide complex is conjugated to one or more desired conjugates, i.e. heterologous moieties, to realize certain functionalities, e.g. to facilitate target detection or for imaging or therapy.

Herein, the present disclosure provides a conjugate, which comprises the polypeptide complex provided herein, and a payload that is conjugated thereto. The payload can be any one of the group consisting of a radioactive label, a fluorescent label, an enzyme-substrate label, an affinity purification tag, a tracer molecule, an anticancer drug, and a cytotoxic molecule.

A variety of conjugates can be linked to the polypeptide complex provided herein by covalent binding, affinity binding, intercalation, coordinate binding, complexation, association, blending, or addition, among others. (see, e.g., “Conjugate Vaccines”, Contributions to Microbiology and Immunology, J. M. Cruse and R. E. Lewis, Jr. (eds.), Carger Press, New York, (1989)).

In certain embodiments, the polypeptide complex provided herein may be engineered to contain specific sites outside the epitope binding portion that may be specifically utilized for binding to one or more conjugates. For example, such a site may include one or more reactive amino acid residues, such as for example cysteine or histidine residues, to facilitate covalent linkage to a conjugate.

In certain embodiments, the N-terminus and/or C-terminus of the polypeptide complex provided herein can also serve to provide reactive groups for conjugation. For example, the N-terminus can be conjugated to one moiety (e.g. polyethylene glycol (PEG), etc.) and the C-terminus is conjugated to another moiety (e.g. biotin, etc.).

In certain embodiments, the polypeptide complex provided herein may be linked to a conjugate directly, or indirectly for example through another conjugate or through a linker.

For example, the polypeptide complex provided herein having a reactive residue such as cysteine may be linked to a thiol-reactive agent in which the reactive group is, for example, a maleimide, an iodoacetamide, a pyridyl disulfide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

For another example, the polypeptide complex provided herein may be conjugated to biotin, then indirectly conjugated to a second conjugate that is conjugated to avidin. For still another example, the polypeptide complex may be linked to a linker which further links to the conjugate. Examples of linkers include bifunctional coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suherate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and his-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio) pentanoate (SPP) to provide for a disulfide linkage.

The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In certain embodiments, the polypeptide complex provided herein may be conjugated to a signal peptide. A signal peptide (sometimes referred to as signal sequence, leader sequence or leader peptide) can be used to facilitate secretion and isolation of the polypeptide complex provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

Methods for the conjugation of conjugates to proteins such as antibodies, immunoglobulins or fragments thereof are found, for example, in U.S. Pat. No. 5,208,020; U.S. Pat. No. 6,4411,163; WO2005037992; WO2005081711; and WO2006/034488, which are incorporated herein by reference to the entirety.

### V. Pharmaceutical Compositions

The present disclosure also provides a pharmaceutical composition. Besides the polypeptide complex as described above, the pharmaceutical composition further comprises a pharmaceutically acceptable carrier.

As used herein, the term “pharmaceutically acceptable” indicates that the designated carrier, vehicle, diluent, excipient(s), salt and/or medium is generally chemically and/or physiologically compatible with other ingredients, such as the active ingredient (i.e. the polypeptide complex or the heterodimeric antibody or antigen-binding fragment thereof) comprising the formulation, and is physiologically compatible with a subject receiving the pharmaceutical composition.

A “pharmaceutically acceptable carrier” refers to an ingredient in a pharmaceutical formulation, other than an active ingredient, which is bioactivity acceptable and nontoxic to a subject. In the context of the present disclosure, a pharmaceutical acceptable carrier for use in the pharmaceutical composition disclosed herein may include, for example, pharmaceutically acceptable liquid, gel or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Herein, suitable “components” may include, for example, antioxidants, fillers, binders, disintegrants, buffers, preservatives, lubricants, flavorings, thickeners, coloring agents, emulsifiers or stabilizers such as sugars and cyclodextrins. Suitable “antioxidants” may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a pharmaceutical composition provided herein decreases oxidation of the polypeptide complex or heterodimeric antibody or antigen-binding fragment thereof. This reduction in oxidation prevents or reduces loss of binding affinity, thereby improving protein stability and maximizing shelf-life. Therefore, in certain embodiments, a pharmaceutical composition is provided that comprise, in addition to the active ingredient (i.e. the polypeptide complex or the heterodimeric antibody or antigen-binding fragment thereof disclosed herein), one or more antioxidants such as methionine.

The pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcellulose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Pharmaceutically acceptable “diluents” may include saline and aqueous buffer solutions.

Pharmaceutically acceptable “adjuvants” may include preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving the polypeptide complex as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the polypeptide complex, the polypeptide complex. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

In certain embodiments, a composition is further provided, comprising a pharmaceutically acceptable carrier, diluent or adjuvant, and an active ingredient. The active ingredient can be the polypeptide complex, antibody or antigen-binding fragment thereof disclosed herein, or the antibody conjugate disclosed herein.

### VI. Preparation Method

The present disclosure provides method for preparing the polypeptide complex provided herein.

The method generally includes the following steps:
(1) providing a nucleic acid that encodes the polypeptide complex;
(2) constructing a vector comprising the nucleic acid;
(3) introducing the vector in a host cell for expression of the polypeptide complex; and
(4) isolating the polypeptide complex from the host cells.

In a first aspect of this section, the present disclosure provides a nucleic acid, which comprises a nucleotide sequence encoding the polypeptide complex disclosed herein.

The nucleic acid encoding the polypeptide complex disclosed herein can be obtained through one of the following approaches.

In a first approach, the nucleic acid encoding the polypeptide complex disclosed herein may be generated from another available nucleic acid that encodes a polypeptide (termed “parent antibody” hereinafter) with a sequence homologous to the polypeptides in the polypeptide complex disclosed herein. Then a DNA manipulation process can be applied to manipulate the sequence of the parent antibody-encoding nucleic acid, such as introducing mutations, insertion, deletion, etc., so as to obtain the nucleic acid encoding the polypeptide complex disclosed herein.

Herein, a “parent antibody” is defined as an antibody or a fragment thereof from which the polypeptide complex disclosed herein can be derived. The parent antibody may have a polypeptide sequence for the heavy chain CH1 region and/or the light chain CL region that is homologous to the heavy chain CH1 region and/or the light chain CL region in the polypeptide complex disclosed herein. The term “homologous” as used herein refers to a situation that a first sequence has a sequences identity of at least 80% (e.g. at least 85%, 88%, 90%, 91%, 92%, 93%, 94%, 95%, 96%, 97%, 98%, 99%) to a second sequence if aligned. Common sequence alignment software is readily available, such as ClustalW (European Bioinformatics Institute website), and the DNA manipulation method is well known in the art, which can include site directed mutagenesis, recombinant DNA techniques, PCR, etc. A parent antibody can be any type, including for example, a fully human antibody, a humanized antibody, or an animal antibody (e.g. mouse, rat, rabbit, sheep, cow, dog, etc.). The antibody can be a monoclonal antibody or a polyclonal antibody.

If the parent antibody-encoding nucleic acid is not available, several different approaches can optionally be applied to obtain the nucleic acid encoding the polypeptide complex disclosed herein. For example, if a host cell (e.g. hybridoma) expressing the parent antibody or a lysate thereof containing mRNAs is available, a reverse transcriptase PCR approach can be applied to obtain cDNAs from the host cell, followed by high-fidelity PCR amplification and sequencing confirmation, to thereby obtain the parent antibody-encoding nucleic acid. If a cDNA library from this parent antibody-host cells is available, only the high-fidelity PCR and sequencing confirmation is needed.

If, however, only the polypeptide sequence of the polypeptide complex disclosed herein is known while the host cell, or the cell lysate or cDNA library thereof is available, the nucleic acid encoding such can alternatively be generated by chemical synthesis, which may include a step of translating the polypeptide sequence into a nucleotide sequence. The knowledge for this task, e.g., nucleotide codons known to encode particular amino acids is well-known in the art.

It is worth a note that the above-mentioned different approaches can be combined to obtain the nucleic acid encoding the polypeptide complex disclosed herein.

In a second aspect of this section, the present disclosure also provides a vector, which comprises the nucleic acid encoding the polypeptide complex disclosed herein.

In this regard, the nucleic acid encoding the polypeptide complex disclosed herein is operably inserted into a vector.

As used herein, the term “vector” refers to a vehicle into which a polynucleotide encoding a protein may be operably inserted so as to bring about the expression of that protein (i.e. called expression vector) and/or to bring about the replication and amplification of the polynucleotide (i.e. called cloning vector) once introduced thereinto. Depending on the different expression systems, a vector can optionally include one or more regulatory sequences, which include a promoter, an enhancer element, a terminator element, a replication origin, or one or more other regulatory elements.

A “promoter” as used herein is a regulatory sequence which is typically upstream of a polypeptide-encoding nucleotide sequence in the vector, and serves to promote the transcription of the target nucleotide sequence through the recognition by a host cell having the vector. The promoter of a vector is typically compatible with the host cell. The promoter may be a bacterial promoter if the host cell is a bacterial expression system, or may be a eukaryotic promoter if the host cell is a eukaryotic expression system. Commonly used promoters are well known in the art.

An “enhancer element”, as used herein in the vector, refers to a special sequence that can enhance the transcription of the target nucleotide sequence when expressed in the host cell. Examples of an enhancer element may include those obtained from mammalian genes (e.g. globin, elastase, albumin, and insulin, etc.), yet may also include eukaryotic cell viruses such as SV40 enhancer, cytomegalovirus early promoter enhancer, polyoma enhancer, and adenovirus enhancer, etc. (see also Yaniv, Nature, 297: 17-18 (1982)). An enhancer element can be located at either 5′- or 3′-end of a polypeptide coding sequence, but is preferably located at a 5′-end position thereof.

A “terminator sequence”, as used herein, refers to a sequence in the vector that is required to terminate transcription and stabilize mRNA. Such a sequence can generally be obtained from a 5′-, sometimes 3′-, or an untranslated region of eukaryotic or viral DNA or cDNA. One specific example of a terminator sequence is in the bovine growth hormone polyadenylation region (for example, see WO 94/11026).

Often, many vectors also comprise a “replication origin”, which is a special nucleic acid sequence enabling these vectors to replicate in the host cells in a manner that is independent from the host chromosomal DNA. Replication origin sequences are well known for many bacteria, yeasts and viruses. Examples of a replication origin include the plasmid pBR322 origin that is suitable for most gram-negative bacteria, the 2 plasmid origin that is suitable for yeasts, and various virus origins (SV40, polyoma, adenovirus, VSV, BPV, etc.) for cloning vectors in mammalian cells. In general, a mammalian expression vector does not need a replication origin (in fact, the SV40 origin is typically often used as a promotor).

Optionally, there are also some other regulatory elements. For example, the 3′-terminal of most eukaryotic genes have an AATAAA sequence which is a signal that adds poly-A to the 3′-terminal of the mRNA, and as such this sequence is commonly found to be inserted into the expression vector of a eukaryotic organism. Others include: a signal sequence, transcription initiation sequences, selectable markers, and reporter genes, etc.

Typically, the target polypeptide-encoding polynucleotide sequence needs to be inserted at an appropriate locus of the vector, such that it is operably linked to the regulatory sequences, such that the expression the target polypeptide is feasible and under proper control.

A vector may be used to transform, transduce, or transfect a host cell so as to bring about expression of the genetic element it carries within the host cell (i.e. “expression vector”) and/or to bring about replication of the vector (“cloning vector”).

Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40).

The encoding polynucleotide sequence(s) can be inserted into a vector for further cloning (amplification of the DNA) or for expression, using recombinant techniques known in the art. In another embodiment, the polypeptide complex and the bispecific polypeptide complex provided herein may be produced by homologous recombination known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

In some embodiments, the vector system includes mammalian, bacterial, yeast systems, etc., and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS420, pLexA, pACT2.2 etc., and other laboratorial and commercially available vectors. Suitable vectors may include, plasmid, or viral vectors (e.g., replication defective retroviruses, adenoviruses and adeno-associated viruses).

In a third aspect of this section, the present disclosure also provides a host cell that comprises the above described nucleic acid, and expresses the polypeptide complex, the antibody or antigen-binding fragment thereof disclosed herein based on the guidance from the nucleic acid.

As used herein, the term “host cell” refers to a cell into which an exogenous polynucleotide (such as the vector described above) has been introduced. Suitable host cells for cloning or expressing the DNA in the vectors can be the prokaryote, yeast, or higher eukaryote cells described above.

Prokaryotes suitable for this purpose include eubacteria that are Gram-negative or Gram-positive organisms. Examples include Escherichia, Enterobacter, Erwinia, Klebsiella, Proteus, Salmonella, etc.

Eukaryotic microbes including filamentous fungi or yeast (Saccharomyces cerevisiae, Schizosaccharomyces pombe, Kluyveromyces, etc.) are suitable cloning or expression hosts for the vector provided herein.

Examples of invertebrate host cells that are suitable for expressing the vector provided herein include plant and insect cells. Numerous baculoviral strains and variants and corresponding permissive insect host cells from hosts such as caterpillars, mosquitos, fruifflies, and Bombyx mori have been identified. Plant cell cultures of cotton, corn, potato, soybean, petunia, tomato, and tobacco can also be utilized as hosts.

Examples of vertebrate host cells, esp. mammalian host cell lines that are suitable for expressing the vector provided herein include monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)), such as Expi293; baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).Vertebrate cells, and propagation of vertebrate cells in culture (tissue culture) has become a routine procedure.

In certain embodiments, an expression vector containing the nucleic acid encoding the polypeptide complex, the heterodimeric antibody or antigen-binding fragment thereof provided herein is introduced into a host cell by transient transfection. As such, the expression and production of the polypeptide complex, the heterodimeric antibody or antigen-binding fragment thereof provided herein in the host cell is transient and does not sustain for a long time. As used herein, the term “transient transfection” refers to a process whereby an exogenous nucleic acid introduced into a host cell does not integrate into the genome or chromosomal DNA of the host cell. After transient transfection, the nucleic acid thus introduced is maintained as an extrachromosomal element (e.g. episome) in the host cell, which can still provide a template for the transcription of the nucleic acid into messenger RNAs (mRNAs) and the subsequent translation of the mRNAs into a polypeptide that the nucleic acid encodes, within the host cell. Therefore, under transient transfection, the production of the polypeptide complex, the heterodimeric antibody or antigen-binding fragment thereof provided herein is only transient and not stable, and usually does not sustain for a long time.

In certain embodiments, in order to realize long-term and high-yield production of the polypeptide complex, the heterodimeric antibody or antigen-binding fragment thereof provided herein, cell lines that stably express the nucleic acid encoding the polypeptide complex, the heterodimeric antibody or antigen-binding fragment thereof provided herein are engineered. To this end, host cells can be transformed with an expression vector comprising a selectable marker. As used herein, the term “selectable marker” refers to a polynucleotide sequence in a vector that encodes a functional polypeptide, such as an enzyme, that provides certain selectivity for the cells stably expressing the expression vector.

In some embodiments, the selectable marker encodes an enzyme that provides resistance to an antibiotic or another toxin (e.g. ampicillin, neomycin, methotrexate or tetracyclin, etc.), which is to be used in a selective medium selecting for the cells expressing the expression vector. More specifically, following transfection of the expression vector, host cells may be allowed to grow for several days in a first culturing media, and then are switched to a selective media containing a corresponding antibiotic/toxin. The selectable marker in the expression vector confers resistance to the selection and allows cells to stably integrate the vector into their chromosomes and grow to form foci which in turn can be cloned and expanded into cell lines. This method is commonly used in the art to engineer cell lines that stably express a foreign protein, and may be useful in screening for cell lines stably expressing the polypeptide complex, the heterodimeric antibody or antigen-binding fragment thereof provided herein.

Optionally, the selectable marker encodes a gene that complements auxotrophic deficiency exhibited by the host cells, and thus can be used to select for cells expressing the expression vector. Further optionally, the selectable marker encodes a gene that supplies an important nutrient that cannot be obtained from (a specific) medium may be given.

Host cells transformed or transfected with the above-described vector can be cultured in conventional nutrient media for expression of the polypeptide complex, the antibody or antigen-binding fragment thereof provided herein, or for amplification of the vector itself.

In a fourth aspect of this section, the present disclosure provides a method of expressing the polypeptide complex disclosed herein.

Generally, the method comprises: culturing the host cell provided herein under an appropriate culturing condition at which the polypeptide complex disclosed herein is expressed.

In some embodiments, a transient expression system is applied, and as such the method includes:
(a) transfecting the host cell with an expression vector configured to express each polypeptide in the polypeptide complex; and
(b) culturing the host cell to express each polypeptide to allow the production of the polypeptide complex.

In some other embodiments, a stable expression system is applied, and as such the method includes:
(a) transfecting the host cell with an expression vector configured to express each polypeptide in the polypeptide complex, wherein the expression vector comprises a selective marker;
(b) obtaining a stable cell line by culturing the transfected host cell in a selective medium corresponding to the selective marker; and
(c) culturing the stable cell line to express each polypeptide to allow the production of the polypeptide complex.

In any of the embodiments above, the host cells transformed with the expression vector described above, or the stable host cell line stably expressing the polypeptide complex provided herein, may be cultured in a variety of media. Examples of commercially available media include Ham's F10 (Sigma), Minimal Essential Medium (MEM), (Sigma), RPMI-1640 (Sigma), and Dulbecco's Modified Eagle's Medium (DMEM, Sigma) are suitable for culturing the host cells. Alternatively, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may also be used as culture media for the host cells.

During culture, any of the above-mentioned media may be further added with one or more supplements as appropriate or per the needs. Non-limiting examples of these supplements include salts (e.g. sodium chloride, calcium, magnesium, and phosphate, etc.), glucose or an equivalent energy source, buffers (e.g. HEPES), nucleotides (e.g. adenosine and thymidine, etc.), selective antibiotics (e.g. GENTAMYCIN™ drug), hormones and/or growth factors (e.g. insulin, transferrin, or epidermal growth factor, etc.), among others. These supplements are to be added at appropriate concentrations that would be known to those skilled in the art. The culture conditions, such as temperature, pH, and the like, are those previously used with the host cell selected for expression, and will be apparent to the ordinarily skilled artisan.

In a fifth aspect of this section, the present disclosure provides a method of isolating the polypeptide complex disclosed herein.

In certain embodiments, the method includes: recovering the polypeptide complex disclosed herein.

As used herein, the term “recover” is considered to be equivalent to “purify,” “separate,” “isolate” and the like, which generally refers to the situation where a molecule of interest (i.e. the polypeptide complex, the heterodimeric antibody or antigen-binding fragment thereof disclosed herein) is enriched, recovered, or separated from a mixture comprising the molecule and other accompanying components in the environment.

Depending on the expression system (i.e. the expression vector and the host cells) used, the polypeptide complex, the heterodimeric antibody or antigen-binding fragment thereof may be produced (termed “desired molecules” hereinafter) within the host cells, in the periplasmic space, or directly secreted into the medium, and the recovering method may include a different first step, as follows.

In certain embodiments where the desired molecules are produced within the cell or intracellularly, the recovering method disclosed herein includes a process of centrifugation or ultrafiltration as its first step, which serves to remove the cell debris including fragments or other unwanted substances.

In some other embodiments where the desired molecules are secreted into a periplasmic space of host cells such as E. Coli, an approach by Carter et al., Bio/Technology (NY) 10: 163-167 (1992) can be applied as its first step. Briefly, a cell paste is cold thawed for about 30 minutes in the presence of sodium acetate (pH 3.5), EDTA and phenylmethylsulfonyl fluoride (PMSF). Then the cell debris can be removed by centrifugation.

In yet some other embodiments where the desired molecules are secreted into the medium, the recovering process may include in its first step: concentrating a supernatant from such an expression system using a protein concentration filter (e.g. Amicon or Pellicon ultrafilter), and this first step and other steps may require the presence of a protease inhibitor (e.g. PMSF) to inhibit antibody degradation, and of an antibiotic to inhibit the growth of exogenous contaminating organisms.

After the first step as described above, a composition prepared from the host cells may be further purified, which can be realized using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments where the desired molecules (i.e. the polypeptide complex, the heterodimeric antibody or antigen-binding fragment thereof disclosed herein) comprises immunoglobulin Fc domain, protein A and/or protein G can be used as the affinity ligand in the affinity chromatography, depending on the species and isotype of the immunoglobulin Fc region present in the molecules. Protein A can be used for affinity-purification of the desired molecules based on human γ1, γ2, or γ4 heavy chains (Lindmark et al., J. Immunol. Meth. 62:1-13 (1983)), whereas protein G can be used for all mouse isotypes and for human γ3 (Guss et al., EMBO J. 5:1567 1575 (1986)).

In other embodiments where the immunoglobulin Fc domain is not present in the desired molecules to the purified, other affinity ligands that can specifically target other epitopes on the desired molecules can also be used for the affinity chromatography. For example, if the desired molecules comprise a CH3 domain, the Bakerbond® ABX resin (J. T. Baker, Phillipsburg, N.J.) can be applied for purification.

In certain embodiments where the desired molecules comprise the immunoglobulin kappa or lambda type light chains, an affinity chromatography matrix (e.g., resins) that are specific therefor (e.g., CaptureSelect Kappa and CaptureSelect Lambda affinity matrices (BAC BV, Holland)) can be used for purification.

Herein, the matrix onto which the affinity ligand is attached typically comprises agarose, but may comprise other materials. Mechanically stable matrices such as controlled pore glass or poly(styrenedivinyl)benzene allow for faster flow rates and shorter processing times than can be achieved with agarose.

Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any of the above preliminary purification step(s), the mixture comprising the desired molecules may be further subjected to low pH hydrophobic interaction chromatography using an elution buffer at a pH between about 2.5-4.5, preferably performed at low salt concentrations (e.g., about 0-0.25M NaCl).

One of the advantages of the desired molecules (i.e. the polypeptide complex, and the heterodimeric antibody or antigen-binding fragment thereof) is that the unwanted mispairing between heavy chain and light chains can be significantly reduced, and therefore typically, production of unwanted byproducts can be minimized even using relatively simple purification processes as described above. As such, it is feasible to obtain high purity product in high yields in certain embodiments.

### VII. Screening and Diagnosis

The polypeptide complex disclosed herein can be used in vivo and/or in vitro for diagnosing or screening diseases that are associated with the antigen(s) targeted thereby.

In one aspect, a method of detecting presence or level of an antigen is provided. The method comprises:
(1) contacting a sample suspected of containing the antigen with the polypeptide complex disclosed herein, and
(2) determining the formation of a complex between the antigen and the polypeptide complex.

In some embodiments, step (1) is typically performed under conditions that allow for formation of a complex between the antigen and the polypeptide complex; and in step (2), the detection of the formation of the complex can be realized using diverse known methodologies, such as ELISA, Western-Blot, FISH assay, immunofluorescence, etc. The sample can be a biological sample, such as plasma, serum, urine, cell lysate, biopsy sample, etc. of a subject suspected to have the disease of interest. The polypeptide complex provided herein is configured to target one or more antigens in the sample that are associated with the disease.

In certain embodiments, the complex formation may be quantified. If the quantity of the target antigen molecules in the sample is higher or lower than a preset threshold, it is determined that the subject may contact the disease.

In certain embodiments, when a control sample is used along with the test sample, the complex formation determination in step (2) may need statistical analysis, where the complex formation is detected and compared in both samples, with a statistically significant difference (e.g. P<0.05) in the formation of complexes between the samples indicative of the presence of the molecule of interest in the test sample. Herein, the control sample may be from a subject absent of the disease, whereas the test sample is suspected to carry the disease.

### VII. Treatment

Depending on the antigens that are specifically targeted, the polypeptide complexes disclosed herein can be used as therapeutic agents to treat a wide range of diseases, conditions, or symptoms.

In one aspect, the present disclosure provides a method for treating or preventing from a disease, condition, or symptom. The method comprises administering to a subject in need thereof a therapeutically effective amount of the polypeptide complex as provided above.

Herein, the polypeptide complex provided herein can be in a pharmaceutical composition as provided above, or can be conjugated to a conjugate as provided above, or can be in a composition as provided above in its therapeutic formulation.

As used herein, the term “subject” or “individual” or “animal” or “patient” refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

As used herein, the term “disorder,” “disease,” “condition” or alike, refers to a condition that affects a subject who would nonetheless benefits from treatment with the polypeptide complex. The term “symptom” refers to a physical, mental, or physiological feature of a patient with a disease, which is regarded as indicating such a condition of the disease.

The term “treatment” is intended to cover both therapeutic treatment and preventative measures, and as such those in need of treatment include those already with the disorder as well as those in which the disorder is to be prevented. As used herein, “treatment” of a condition may include: preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combinations thereof.

As used herein, the term “therapeutically effective amount” of a therapeutic agent refers to an amount of the therapeutic agent that, when taken by a subject in an appropriate manner, can generate sufficient therapeutic effects to the subject. It is to be understood that just like other therapeutic drugs, the therapeutically effective amount of the polypeptide complex as provided above will be influenced by various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the polypeptide complex provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the polypeptide complex or the bispecific polypeptide complex provided herein is administered at a dosage of about 50 mg/kg or less, and in certain of these embodiments the dosage is 10 mg/kg or less, 5 mg/kg or less, 1 mg/kg or less, 0.5 mg/kg or less, or 0.1 mg/kg or less. In certain embodiments, the administration dosage may be adjusted over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The polypeptide complex provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

The polypeptide complex provided herein may be administered alone or in combination with one or more additional therapeutic means or agents.

Herein, the polypeptide complex, and the heterodimeric antibody or antigen-binding fragment thereof described above and the method disclosed herein can be applied to treat a wide variety of diseases. In humans, and other primates as well, the diseases that are contemplated to be treatable by the polypeptide complex, and the heterodimeric antibody or antigen-binding fragment thereof described above and the method disclosed herein can include the following:
(1) cancers and other hyperproliferative disorders, including both benign or malignant tumors, leukemia and lymphoid malignancies. Depending on the cell type having cancers or hyperproliferative disorders, examples include neuronal, glial, astrocytal, hypothalamic, glandular, macrophagal, epithelial, endothelial, and stromal malignancies. Depending on the organ/location afflicted with cancers or hyperproliferative disorders, examples include: cancers of the head, neck, eye, mouth, throat, esophagus, chest, skin, bone, lung, colon, rectum, colorectal, stomach, spleen, kidney, skeletal muscle, subcutaneous tissue, metastatic melanoma, endometrial, prostate, breast, ovaries, testicles, thyroid, blood, lymph nodes, kidney, liver, pancreas, brain, or central nervous system;
(2) autoimmune and/or inflammatory disorders, including alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, Sjogren's syndrome, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation, sepsis, rheumatoid arthritis, peritonitis, Crohn's disease, reperfusion injury, septicemia, endotoxic shock, cystic fibrosis, endocarditis, psoriasis, arthritis (e.g., psoriatic arthritis), anaphylactic shock, organ ischemia, reperfusion injury, spinal cord injury and allograft rejection. autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitisherpetiformis vasculitis, vitiligo, and Wegener's granulomatosis. Inflammatory disorders, can further include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections;
(3) infectious and parasitic diseases, such as those caused by viruses (e.g. HBV, HCV, HIV, RSV, hMPV, PIV, coronaviruses, or influenza viruses, etc.), fungi (e.g. Naegleria, Aspergillus, Blastomyces, Histoplasma, Candida or Tinea genera, etc.), eukaryotic microbes (e.g. Giardia, Toxoplasma, Plasmodium, Trypanosoma, and Entamoeba genera, etc.), and bacteria (Staphylococcus, Streptococcus, Pseudomonas, Clostridium, Borrelia, Vibro and Neiserria genera, etc.);
(4) other disease or disorders, including those not covered by an of the above in (1)-(3), such as cardiovascular diseases, neuropathies, neuropsychiatric conditions, injuries, or coagulation disorder, etc.

Antigens associated with the above listed diseases that can be treated by the polypeptide complex as described above, or through the treatment method disclosed herein include the following:
(1) tumor associated antigens, which refers to antigens presented on a tumor cell surface, located on or within tumor cells, presented only by tumor cells and not by normal, i.e. non-tumor cells, representing a protein harboring one or more tumor-specific mutations compared to non-tumor cells, overexpressed in tumor cells when compared to non-tumor cells; accessible for antibody binding in tumor cells due to the less compact structure of the tumor tissue compared to non-tumor tissue, and presented on the vasculature of a tumor, etc. Examples include, but are not limited to: CD19, CD20, CD38, CD30, Her2/neu/ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin a5p1, integrin avP3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin, etc.
(2) antigens associated with autoimmune diseases or inflammatory diseases, including but not limited to, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin a4, integrin a407, LFA-1 (CD11a), myostatin, OX-40, scleroscin, SOST, TGF beta 1, TNF-α, and VEGF-A, etc.

The heterodimeric antibody or antigen-binding fragment thereof, as disclosed herein, can specifically targets two or more antigens simultaneously, which can be used for the effective treatment of certain diseases.

In some preferred embodiments of the heterodimeric antibody or antigen-binding fragment thereof provided herein, one antigen-binding moiety targets a receptor on the cytotoxic T lymphocytes (e.g. CD3), the other antigen-binding moiety targets one of following tumor cell-expressing antigens: CD19, CD20, CD33, CD123, HER1, HER2, CEA, disialoganglioside GD2, PSMA, gpA33, EpCAM, P-cadherin, and B7H3 (Sedykh S E, et al., Drug Des Devel Ther. 2018; 12: 195-208.). As such, the bispecific antibody or its antigen-binding fragment thereof can be used to form a link between T cells and tumor cells, which can cause the T cells to exert cytotoxic activity on tumor cells.

Other examples of antigen pairs that can be targeted by the heterodimeric antibody or antigen-binding fragment thereof to thereby have potential therapeutic effects can include, but are not limited to, PD-L1:TGFβ, CD38:EGFR, HER2:VEGF, HER2:EGFR, PD-1:CTLA-4, PD-1:TIM3, OX40:PD-L1, FIXa:FX, CD32B:CD79B, Angiopoietin 2:VEGF, IL13:IL4, TNF:IL17A, DLL4:VEGF, IL1α:IL1β, FAP:DR5, CD30:gpA33, TNF:HSA, IL6R:HSA, IL17A/F:HSA, RANKL:HSA, A040:AP42, IL13:IL17, FGFRI:KLB, PsI:PcrV, BAFF:B7RP1, NGF:TNF, and TNF:IL17A (Sedykh S E, et al., Drug Des Devel Ther. 2018; 12: 195-208).

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EMBODIMENTS

### Example 1: CH-CL Interface Study

The disulfide bond formed by C220 of CH1 (C131 for IgG2 and IgG4) and C214 (Kappa chain or Lambda chain) of CL is the basis for the association between CH1 and CL regions. Therefore, if the disulfide bond association mode between the CH1 and CL regions of LC(B)-HC(B) is changed, the LC(A)-HC(B) and LC(B)-HC(A) mispairings may be avoided, such that the four chains, i.e. LC(A), HC(A), HC(B) and LC(B), can be expressed in one cell line, thereby simplifying the expression and purification process.

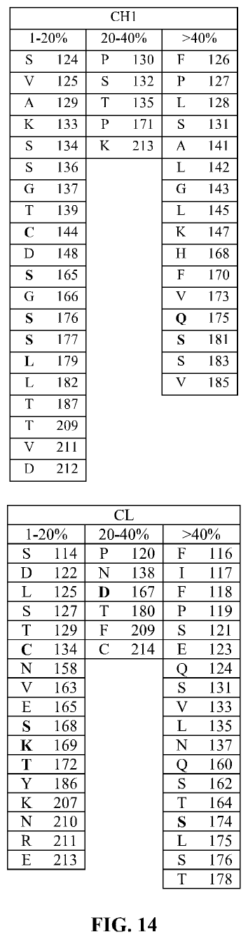

In order to change the association mode of LC(B)-HC(B), the inventors studied the CH-CL interface, listed the relevant amino acids, and analyzed their respective ratio in the interface (see FIG. 14), which would be used as the basis for the subsequent construction.

### Example 2: CH1-VH and CL-VL Interfaces Study

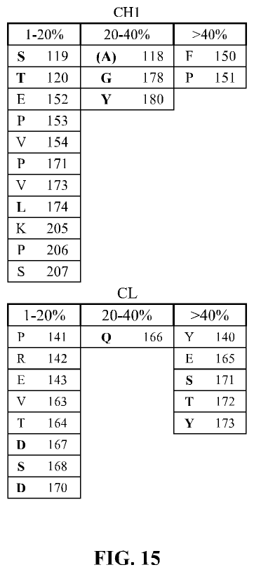

Because CH1 and CL regions shall be associated with VH and VL regions, respectively, there will be CH1-VH and CL-VL interfaces as well. In order to maintain the spatial structures of the original VH and VL regions as much as possible, and maintain stability, while avoiding the mutation site exposed to the hydrophilic surface of CH1 and CL regions (which may lead to changes in the druggability and immunogenicity of the antibody), the inventors analyzed CH1-VH and CL-VL interfaces, and the results were shown in FIG. 15. The binding ratio of related amino acids was also analyzed to exclude some amino acids involved in the interface of the constant region and the variable region.

### Example 3: Distance Measurement

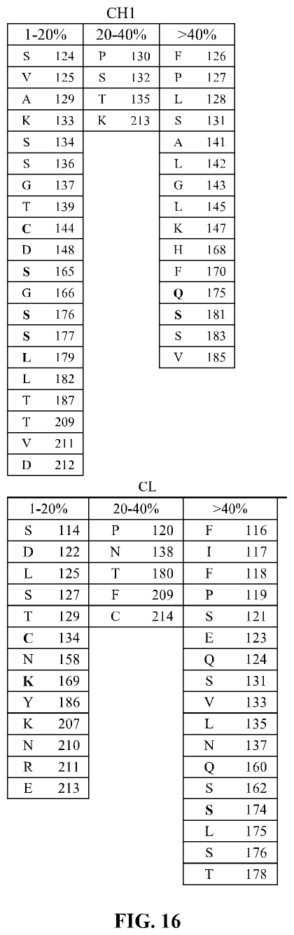

The residues that can be used for mutation study were selected by analyzing FIG. 14 and FIG. 15. The results were shown in FIG. 16. The residues that having a binding area more than 40% were analyzed, and software was used to measure the distances of these residues on CH1 and CL regions. the results were shown in Table 1 below.

TABLE 1
The distances of residues for mutations in CH1 and CL regions

| CH1 | | | CL | |
|---|---|---|---|---|
| Eu Numbering | Residue | Cβ-Cβ(Å) | Eu Numbering | Residue |
| 141 | A | 4.12 | 116 | F |
| 128 | L | 4.24 | 118 | F |
| 126 | F | 4.71 | 121 | S |
| 126 | F | 5.87 | 123 | E |
| 187 | T | 5.73 | 137 | N |
| 183 | S | 4.38 | 176 | S |
| 183 | S | 6.97 | 178 | T |
| 131 | C | 4.77 | 119 | P |
| 173 | V | 4.35 | 160 | Q |
| 128 | L | 5.65 | 133 | V |
| 168 | H | 4.71 | 174 | S |

### Example 4: Calculation

In order to avoid the formation of disulfide bond between the mutated residues and C220 (CH1) and C214 (CL), L128-F118 and V173-Q160 were selected for mutation through comprehensive analysis. Based on this, a novel CH1-CL binding mode was constructed to avoid the formation of mispairing with wild-type CH1/CL.

### Example 5: Expression of Bispecific Antibodies Based on L128C-F118C or V173C-Q160C Construct

The sequences as shown in Table 2 below were taken as templates for constructing bispecific antibodies. In Table 1 below, “Antibody A” refers to “anti-PD1 antibody”, “Antibody B” refers to “anti-TGFβ3R antibody”, “HC(A)” refers to the heavy chain of Antibody A, “LC(A)” refers to the light chain of Antibody A, “HC(B)” refers to the heavy chain of Antibody B, “LC(B)” refers to the light chain of Antibody B.

TABLE 2
Amino acid sequences of the heavy chains and light chains of
Antibody A and Antibody B

| | | SEQ ID |
|---|---|---|
| Name | Amino Acid Sequence | NO. |
| HC(A) | QVQLVESGGGVVQPGRSLRLDCKASGITFSNSGMHWVRQAPGKGLE | 1 |
| | WVAVIWYDGSKRYYADSVKGRFTISRDNSKNTLFLQMNSLRAEDTA | |
| | VYYCATNDDYWGQGTLVTVSSASTKGPSVFPLAPSSKSTSGGTAAL | |
| | GCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEL | |
| | LGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQ | |
| | QGNVFSCSVMHEALHNHYTQKSLSLSPGK | |
| LC(A) | EIVLTQSPATLSLSPGERATLSCRASQSVSSYLAWYQQKPGQAPRL | 2 |
| | LIYDASNRATGIPARFSGSGSGTDFTLTISSLEPEDFAVYYCQQSS | |
| | NWPRTFGQGTKVEIKRTVAAPSVFIFPPSDEQLKSGTASVVCLLNN | |
| | FYPREAKVQWKVDNALQSGNSQESVTEQDSKDSTYSLSSTLTLSKA | |
| | DYEKHKVYACEVTHQGLSSPVTKSFNRGEC | |
| HC(B) | QVQLVQSGAEVKKPGSSVKVSCKASGYTFSSNVISWVRQAPGQGLE | 3 |
| | WMGGVIPIVDIANYAQRFKGRVTITADESTSTTYMELSSLRSEDTA | |
| | VYYCASTLGLVLDAMDYWGQGTLVTVSSASTKGPSVFPLAPSSKST | |
| | SGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCP | |
| | PCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEY | |
| | KCKVSNKALPAPIEKTISKAKGQPREPQVYTLPPSREEMTKNQVSL | |
| | TCLVKGFYPSDIAVEWESNGQPENNYKTTPPVLDSDGSFFLYSKLT | |
| | VDKSRWQQGNVFSCSVMHEALHNHYTQKSLSLSPGK | |
| LC(B) | ETVLTQSPGTLSLSPGERATLSCRASQSLGSSYLAWYQQKPGQAPR | 4 |
| | LLIYGASSRAPGIPDRESGSGSGTDFTLTISRLEPEDFAVYYCQQY | |
| | ADSPITFGQGTRLEIKRTVAAPSVFIFPPSDEQLKSGTASVVCLLN | |
| | NFYPREAKVQWKVDNALQSGNSQESVTEQDSKDSTYSLSSTLTLSK | |
| | ADYEKHKVYACEVTHQGLSSPVTKSFNRGEC | |

To prevent HC(A) and HC(B) from forming homodimers, K392D and K409D mutations were introduced into CH3 of HC(A), D399K and E356K mutations were introduced into CH3 of HC(B). The sequences of HC(A) and HC(B) with mutations in CH3 domains are shown in Table 3 below, and the mutated amino acids were underlined.

TABLE 3
Amino acid sequences of HC(A) and HC(B) with mutations in CH3 domains.

| | | SEQ ID |
|---|---|---|
| Name | Amino Acid Sequence | NO. |
| HC(A) | QVQLVESGGGVVQPGRSLRLDCKASGITFSNSGMHWVRQAPG | 5 |
| (K392D:K409D) | KGLEWVAVIWYDGSKRYYADSVKGRFTISRDNSKNTLFLQMN | |
| | SLRAEDTAVYYCATNDDYWGQGTLVTVSSASTKGPSVFPLAP | |
| | SSKSTSGGTAALGCLVKDYFPEPVTVSWNSGALTSGVHTFPA | |
| | VLQSSGLYSLSSVVTVPSSSLGTQTYICNVNHKPSNTKVDKK | |
| | VEPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAK | |
| | GQPREPQVYTLPPSREEMTKNQVSLTCLVKGFYPSDIAVEWE | |
| | SNGQPENNYDTTPPVLDSDGSFFLYSDLTVDKSRWQQGNVES | |
| | CSVMHEALHNHYTQKSLSLSPGK | |
| HC(B) | QVQLVQSGAEVKKPGSSVKVSCKASGYTFSSNVISWVRQAPG | |
| (D399K:E356K) | QGLEWMGGVIPIVDIANYAQRFKGRVTITADESTSTTYMELS | |
| | SLRSEDTAVYYCASTLGLVLDAMDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | 6 |
| | TLMISRTPEVTCVVVDVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPSRKEMTKNQVSLTCLVKGFYPS | |
| | DIAVEWESNGQPENNYKTTPPVLKSDGSFFLYSKLTVDKSRW | |
| | QQGNVFSCSVMHEALHNHYTQKSLSLSPGK | |

In order to evaluate the effects of different CH1-CL interface residue mutations on mispairing, antibodies DIC14, DIC002 and DIC007 were constructed. As shown in Table 4 below, for DIC014, no mutation was introduced to the CH1 and CL regions; for DIC02, L128C and C220S mutations were introduced into the CH(A) region, F118C and C214S mutations were introduced into the LC(A) region, and no mutations were introduced into the CH1(B) and LC(B) regions; for DIC007, V173C and C220S mutations were introduced into the CH1(A) region, Q160C and C214S mutations were introduced into the LC(A) region, and no mutations were introduced into the CH1 (B) and LC(B) regions.

TABLE 4
Mutations in CH1 and CL regions of exemplary antibodies

| Antibody Name | CH1(A) | LC(A) | CH1(B) | LC(B) |
|---|---|---|---|---|
| DIC014 | — | — | — | — |
| DIC002 | L128C, C220S | F118C, C214S | — | — |
| DIC007 | V173C, C220S | Q160C, C214S | — | — |

The sequence information of LC(A), HC(A), LC(B) and HC(B) regions of DIC014, DIC002 and DIC007 are shown in Table 5 below. As shown in Table 5, DIC014 comprises the LC(A), HC(A), LC(B) and HC(B) regions as set forth in SEQ ID NO: 2, 7, 4 and 8, respectively; DIC002 comprises the LC(A), HC(A), LC(B) and HC(B) regions as set forth in SEQ ID NO: 9, 10, 4 and 8, respectively; DIC007 comprises the LC(A), HC(A), LC(B) and HC(B) regions as set forth in SEQ ID NO: 30, 11, 4 and 8, respectively.

TABLE 5
Constant region sequences of the heavy chains and light chains of exemplary
antibodies

| Antibody | | | SEQ |
|---|---|---|---|
| Name | | Amino Acid Sequence | ID NO. |
| DIC014 | LC(A) | EIVLTQSPATLSLSPGERATLSCRASQSVSSYLAW | 2 |
| | | YQQKPGQAPRLLIYDASNRATGIPARFSGSGSGTD | |
| | | FTLTISSLEPEDFAVYYCQQSSNWPRTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |
| | HC(A) | QVQLVESGGGVVQPGRSLRLDCKASGITFSNSGMH | 7 |
| | | WVRQAPGKGLEWVAVIWYDGSKRYYADSVKGRFTI | |
| | | SRDNSKNTLFLQMNSLRAEDTAVYYCATNDDYWGQ | |
| | | GTLVTVSSASTKGPSVFPLAPSSKSTSGGTAALGC | |
| | | LVKDYFPEPVTVSWNSGALTSGVHTFPAVLQSSGL | |
| | | YSLSSVVTVPSSSLGTQTYICNVNHKPSNTKVDKK | |
| | | VEPKSCDKTHTCPPCPAPPVAGPSVELFPPKPKDT | |
| | | LMISRTPEVTCVVVDVSQEDPEVKFNWYVDGVEVH | |
| | | NAKTKPREEQFNSTYRVVSVLTVLHQDWLNGKEYK | |
| | | CKVSNKGLPSSIEKTISKAKGQPREPQVYTLPPSR | |
| | | DELTKNQVSLTCLVKGFYPSDIAVEWESNGQPENN | |
| | | YDTTPPVLDSDGSFFLYSDLTVDKSRWQQGNVFSC | |
| | | SVMHEALHNHYTQKSLSLSP | |
| | LC(B) | ETVLTQSPGTLSLSPGERATLSCRASQSLGSSYLA | 4 |
| | | WYQQKPGQAPRLLIYGASSRAPGIPDRESGSGSGT | |
| | | DFTLTISRLEPEDFAVYYCQQYADSPITFGQGTRL | |
| | | EIKRTVAAPSVFIFPPSDEQLKSGTASVVCLLNNE | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| | HC(B) | QVQLVQSGAEVKKPGSSVKVSCKASGYTFSSNVIS | 8 |
| | | WVRQAPGQGLEWMGGVIPIVDIANYAQRFKGRVTI | |
| | | TADESTSTTYMELSSLRSEDTAVYYCASTLGLVLD | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPPVAGPSVELF | |
| | | PPKPKDTLMISRTPEVTCVVVDVSQEDPEVKFNWY | |
| | | VDGVEVHNAKTKPREEQFNSTYRVVSVLTVLHQDW | |
| | | LNGKEYKCKVSNKGLPSSIEKTISKAKGQPREPQV | |
| | | YTLPPSRKELTKNQVSLTCLVKGFYPSDIAVEWES | |
| | | NGQPENNYKTTPPVLKSDGSFFLYSKLTVDKSRWQ | |
| | | QGNVFSCSVMHEALHNHYTQKSLSLSP | |
| DIC002 | LC(A) | EIVLTQSPATLSLSPGERATLSCRASQSVSSYLAW | 9 |
| | | YQQKPGQAPRLLIYDASNRATGIPARFSGSGSGTD | |
| | | FTLTISSLEPEDFAVYYCQQSSNWPRTFGQGTKVE | |
| | | IKRTVAAPSVFICPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGES | |
| | HC(A) | QVQLVESGGGVVQPGRSLRLDCKASGITESNSGMH | 10 |
| | | WVRQAPGKGLEWVAVIWYDGSKRYYADSVKGRFTI | |
| | | SRDNSKNTLFLQMNSLRAEDTAVYYCATNDDYWGQ | |
| | | GTLVTVSSASTKGPSVFPCAPSSKSTSGGTAALGC | |
| | | LVKDYFPEPVTVSWNSGALTSGVHTFPAVLQSSGL | |
| | | YSLSSVVTVPSSSLGTQTYICNVNHKPSNTKVDKK | |
| | | VEPKSSDKTHTCPPCPAPPVAGPSVFLFPPKPKDT | |
| | | LMISRTPEVTCVVVDVSQEDPEVKFNWYVDGVEVH | |
| | | NAKTKPREEQFNSTYRVVSVLTVLHQDWLNGKEYK | |
| | | CKVSNKGLPSSIEKTISKAKGQPREPQVYTLPPSR | |
| | | DELTKNQVSLTCLVKGFYPSDIAVEWESNGQPENN | |
| | | YDTTPPVLDSDGSFFLYSDLTVDKSRWQQGNVFSC | |
| | | SVMHEALHNHYTQKSLSLSP | |
| | LC(B) | ETVLTQSPGTLSLSPGERATLSCRASQSLGSSYLA | 4 |
| | | WYQQKPGQAPRLLIYGASSRAPGIPDRESGSGSGT | |
| | | DFTLTISRLEPEDFAVYYCQQYADSPITFGQGTRL | |
| | | EIKRTVAAPSVFIFPPSDEQLKSGTASVVCLLNNE | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| | HC(B) | QVQLVQSGAEVKKPGSSVKVSCKASGYTFSSNVIS | 8 |
| | | WVRQAPGQGLEWMGGVIPIVDIANYAQRFKGRVTI | |
| | | TADESTSTTYMELSSLRSEDTAVYYCASTLGLVLD | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPPVAGPSVELF | |
| | | PPKPKDTLMISRTPEVTCVVVDVSQEDPEVKFNWY | |
| | | VDGVEVHNAKTKPREEQFNSTYRVVSVLTVLHQDW | |
| | | LNGKEYKCKVSNKGLPSSIEKTISKAKGQPREPQV | |
| | | YTLPPSRKELTKNQVSLTCLVKGFYPSDIAVEWES | |
| | | NGQPENNYKTTPPVLKSDGSFFLYSKLTVDKSRWQ | |
| | | QGNVFSCSVMHEALHNHYTQKSLSLSP | |
| DIC007 | LC(A) | EIVLTQSPATLSLSPGERATLSCRASQSVSSYLAW | 30 |
| | | YQQKPGQAPRLLIYDASNRATGIPARFSGSGSGTD | |
| | | FTLTISSLEPEDFAVYYCQQSSNWPRTFGQGTKVE | |
| | | IKRTVAAPSVFIFPPSDEQLKSGTASVVCLLNNFY | |
| | | PREAKVQWKVDNALQSGNSCESVTEQDSKDSTYSL | |
| | | SSTLTLSKADYEKHKVYACEVTHQGLSSPVTKSFN | |
| | | RGES | |
| | HC(A) | QVQLVESGGGVVQPGRSLRLDCKASGITFSNSGMH | 11 |
| | | WVRQAPGKGLEWVAVIWYDGSKRYYADSVKGRFTI | |
| | | SRDNSKNTLFLQMNSLRAEDTAVYYCATNDDYWGQ | |
| | | GTLVTVSSASTKGPSVFPLAPSSKSTSGGTAALGC | |
| | | LVKDYFPEPVTVSWNSGALTSGVHTFPACLQSSGL | |
| | | YSLSSVVTVPSSSLGTQTYICNVNHKPSNTKVDKK | |
| | | VEPKSSDKTHTCPPCPAPPVAGPSVFLFPPKPKDT | |
| | | LMISRTPEVTCVVVDVSQEDPEVKFNWYVDGVEVH | |
| | | NAKTKPREEQFNSTYRVVSVLTVLHQDWLNGKEYK | |
| | | CKVSNKGLPSSIEKTISKAKGQPREPQVYTLPPSR | |
| | | DELTKNQVSLTCLVKGFYPSDIAVEWESNGQPENN | |
| | | YDTTPPVLDSDGSFFLYSDLTVDKSRWQQGNVFSC | |
| | | SVMHEALHNHYTQKSLSLSP | |
| | LC(B) | ETVLTQSPGTLSLSPGERATLSCRASQSLGSSYLA | 4 |
| | | WYQQKPGQAPRLLIYGASSRAPGIPDRESGSGSGT | |
| | | DFTLTISRLEPEDFAVYYCQQYADSPITFGQGTRL | |
| | | EIKRTVAAPSVFIFPPSDEQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| | HC(B) | QVQLVQSGAEVKKPGSSVKVSCKASGYTFSSNVIS | 8 |
| | | WVRQAPGQGLEWMGGVIPIVDIANYAQRFKGRVTI | |
| | | TADESTSTTYMELSSLRSEDTAVYYCASTLGLVLD | |
| | | AMDYWGQGTLVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPS | |
| | | NTKVDKKVEPKSCDKTHTCPPCPAPPVAGPSVFLF | |
| | | PPKPKDTLMISRTPEVTCVVVDVSQEDPEVKFNWY | |
| | | VDGVEVHNAKTKPREEQFNSTYRVVSVLTVLHQDW | |
| | | LNGKEYKCKVSNKGLPSSIEKTISKAKGQPREPQV | |
| | | YTLPPSRKELTKNQVSLTCLVKGFYPSDIAVEWES | |
| | | NGQPENNYKTTPPVLKSDGSFFLYSKLTVDKSRWQ | |
| | | QGNVFSCSVMHEALHNHYTQKSLSLSP | |

### a) Expression

The DNA sequences encoding four chains of DIC014, DIC002 and DIC007 were cloned into vectors (at a ratio of 1:1:1:1), and transfected into ExpiCHO cells for transient expression, and then analyzed after affinity chromatography purification.

### b) SDS-PAGE and SEC-HPLC Analysis

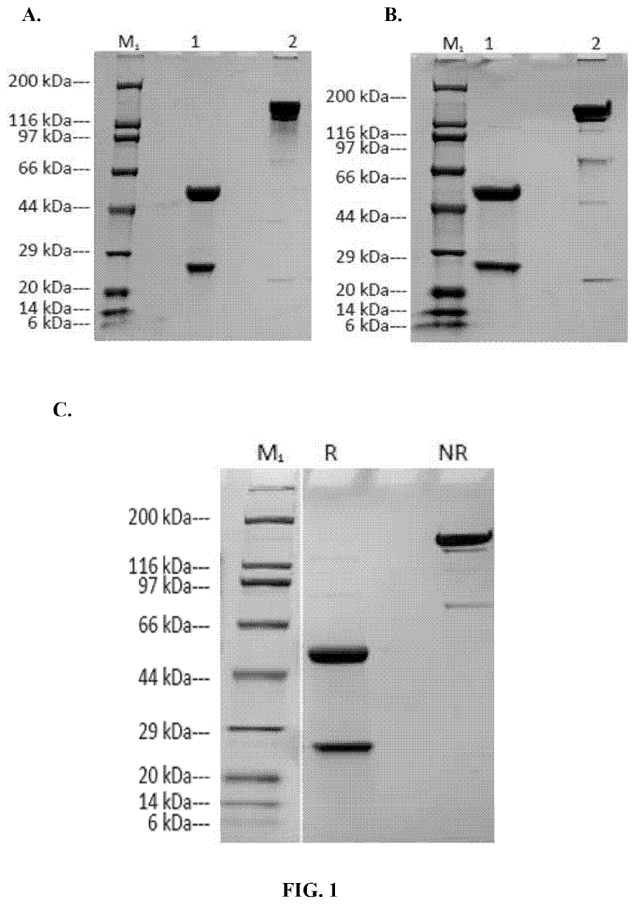

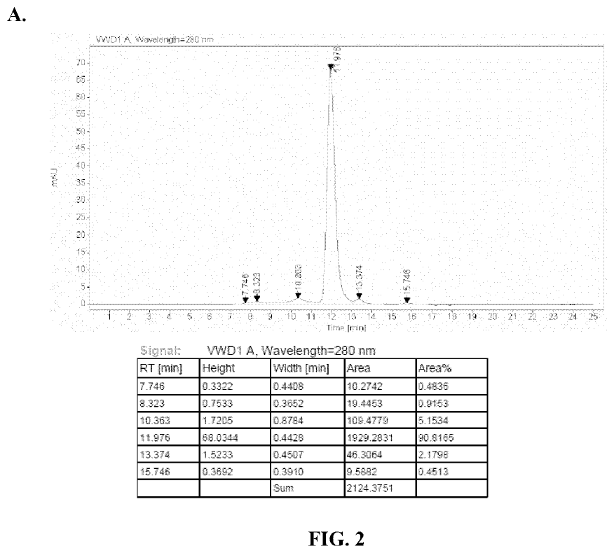

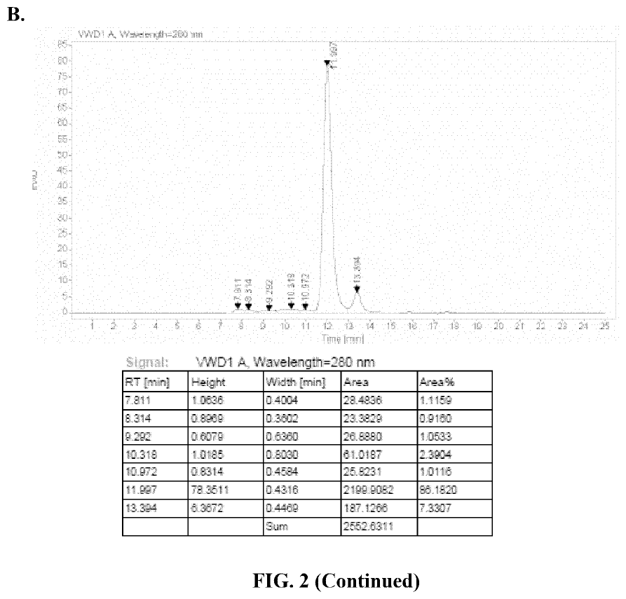

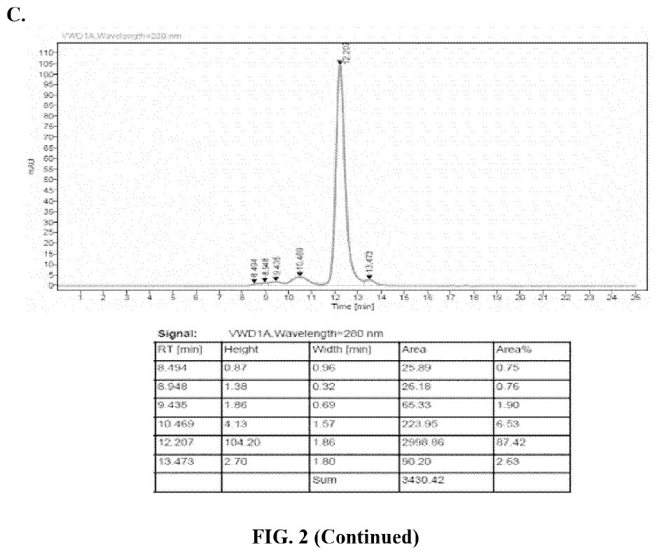

The purified antibodies above were detected by SDS-PAGE and SEC-HPLC, respectively. The SDS-PAGE results are shown in FIG. 1A (DIC002), FIG. 1B (DIC007), and FIG. 1C (DIC014). The SEC-HPLC results are shown in FIG. 2A (DIC002), FIG. 2B (DIC007), and FIG. 2C (DIC014).

Since the CL and CH on the left and right sides are associated in different ways, if HC(A)-LC(B) and/or HC(B)-LC(A) mispairings occur during the construction of the asymmetric antibodies above, such mispairings cannot form a stable disulfide bond linkage, which would result in small molecule bands such as 125 KDa and 100 KDa in SDS-PAGE, as well as shoulder peaks and/or small peaks on the right side of the main peak in SEC-HPLC.

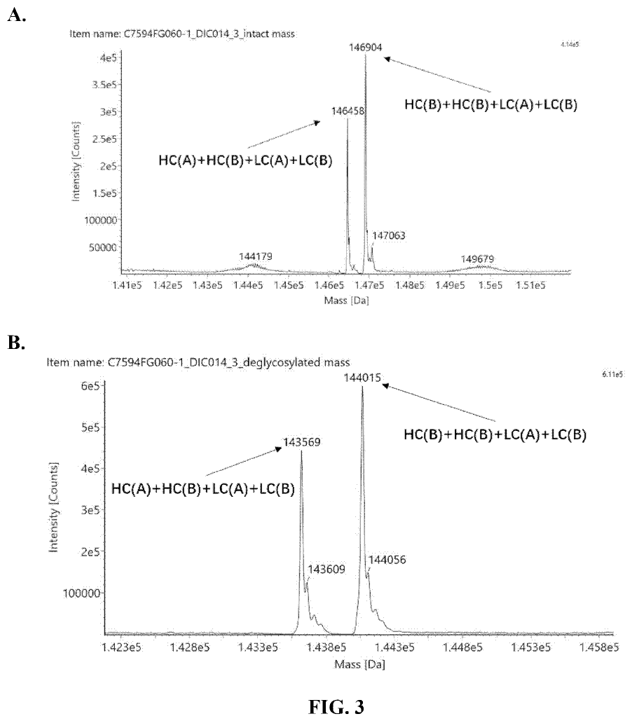

Regarding DIC014, because both HC(A)-LC(A) and HC(B)-LC(B) are connected by C220(CH1)-C214(CL) to form disulfide bond linkages, the HC(A)-LC(B) and HC(B)-LC(A) mispairings formed in the expression products are also covalently bound, and thus the performance of DIC014 in SDS-PAGE and SEC-HPLC was similar to that of ordinary antibodies. Therefore, DIC014 was analyzed by LC-MS, which identified a high mispairing ratio, and the main mispairinged type is HC(B)-HC(B)-LC(A)-LC(B). The LC-MS results of intact DIC014 and deglycosylated DIC014 were shown in FIG. 3A (intact DIC014) and FIG. 3B (deglycosylated DIC014). The results showed that HC(A)-LC(B) and/or HC(B)-LC(A) mispairings would occur during the constructions of DIC002, DIC007 and DIC014.

### Example 6: Charges were Introduced to Reduce Mispairings in Bispecific Antibodies Constructed Based on L128C-F118C or V173C-Q160C

HC(A)-LC(B) and/or HC(B)-LC(A) mispairings would still occur in the bispecific antibodies constructed based on L128C-F1118C or V173C-Q160C. In order to reduce the mispairing ratio, the inventors adjusted the charges between the interface of CH1(A)-LC(A) and CH1(B)-LC(B), in addition to the L128C-F1118C or V173C-Q160C mutations.

Charge mutations were introduced, in addition to L128C-F1118C or V173C-Q160C mutations, by modeling the protein structures of CH1-CL. As shown in Table 6 below, if a positively charged amino acid (such as R, H or K) was introduced to one residue, then a negatively charged amino acid (such as D or E) was introduced into the other residue. For example, for No. 1 in Table 6, if a positively charged amino acid (such as R, H or K) was introduced into S183 of CH1(A), then a negatively charged amino acid (such as D or E) was introduced to S176 of LC(A).

TABLE 6
Mutated residues in CH1(A) and LC(A)

| No. | CH1(A) | LC(A) |
|---|---|---|
| 1 | S183 | S176 |
| 2 | F126 | S121 |
| 3 | A141 | F116 |
| 4 | K218 | D122 |

Because some residue mutations would affect the CH1-CL disulfide bond linkage, it was found, after calculation, that V173C-Q160C mutations could not increase F126-S121 pairing mutations. Therefore, the mutation combinations as shown in Table 7 below were analyzed (the positively charged and negatively charged residues can be interchanged). As shown in Table 7, no mutation was introduced to CH1(B) and LC(B) regions of each antibody. For DIC003, L128C, C220S and S183D mutations were introduced into the CH1(A) region, F118C, C214S and S176K mutations were introduced into the LC(A) region; for DIC004, L128C, C220S and F126D mutations were introduced into the CH1(A) region, F118C, C214S and S121K mutations were introduced into the LC(A) region; for DIC005, L128C, C220S and A141D mutations were introduced into the CH1(A) region, F118C, C214S and F116K mutations were introduced into the LC(A) region; for DIC006, L128C, C220S and K218D mutations were introduced into the CH1(A) region, F118C, C214S and D122K mutations were introduced into the LC(A) region; for DIC009, V173C, C220S and S183D mutations were introduced to the CH1(A) region, Q160C, C214S and S176K mutations were introduced to the LC(A) region; for DIC010, V173C, C220S and A141D mutations were introduced to the CH1(A) region, Q160C, C214S and F116K mutations were introduced to the LC(A) region.

TABLE 7
Mutations in CH1 and CL regions of exemplary antibodies

| Antibody | | | | |
|---|---|---|---|---|
| Name | CH1(A) | LC(A) | CH1(B) | LC(B) |
| DIC003 | L128C, C220S | F118C, C214S | — | — |
| | S183D | S176K | — | — |
| DIC004 | L128C, C220S | F118C, C214S | — | — |
| | F126D | S121K | — | — |
| DIC005 | L128C, C220S | F118C, C214S | — | — |
| | A141D | F116K | — | — |
| DIC006 | L128C, C220S | F118C, C214S | — | — |
| | K218D | D122K | — | — |
| DIC009 | V173C, C220S | Q160C, C214S | — | — |
| | S183D | S176K | — | — |
| DIC010 | V173C, C220S | Q160C, C214S | — | — |
| | A141D | F116K | — | — |

The sequence information of LC(A), HC(A), LC(B) and HC(B) regions of DIC003, DIC004, DIC005, DIC006, DIC009 and DIC010 are shown in Table 8 below.

TABLE 8
Constant region sequences of the heavy chains and light chains of exemplary
antibodies

| Antibody | | | SEQ |
|---|---|---|---|
| Name | | Amino Acid Sequence | ID NO. |
| DIC003 | LC(A) | EIVLTQSPATLSLSPGERATLSCRASQSVSSYLAWY | 12 |
| | | QQKPGQAPRLLIYDASNRATGIPARFSGSGSGTDFT | |
| | | LTISSLEPEDFAVYYCQQSSNWPRTFGQGTKVEIKR | |
| | | TVAAPSVFICPPSDEQLKSGTASVVCLLNNFYPREA | |
| | | KVQWKVDNALQSGNSQESVTEQDSKDSTYSLESTLT | |
| | | LSKADYEKHKVYACEVTHQGLSSPVTKSFNRGES | |
| | HC(A) | QVQLVESGGGVVQPGRSLRLDCKASGITFSNSGMHW | 13 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVFPCAPSSKSTSGGTAALGCLVKD | |
| | | YFPEPVTVSWNSGALTSGVHTFPAVLQSSGLYSLRS | |
| | | VVTVPSSSLGTQTYICNVNHKPSNTKVDKKVEPKSS | |
| | | DKTHTCPPCPAPPVAGPSVFLFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRESGSGSGTDE | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |
| DIC004 | LC(A) | EIVLTQSPATLSLSPGERATLSCRASQSVSSYLAWY | 14 |
| | | QQKPGQAPRLLIYDASNRATGIPARFSGSGSGTDFT | |
| | | LTISSLEPEDFAVYYCQQSSNWPRTFGQGTKVEIKR | |
| | | TVAAPSVFICPPEDEQLKSGTASVVCLLNNFYPREA | |
| | | KVQWKVDNALQSGNSQESVTEQDSKDSTYSLSSTLT | |
| | | LSKADYEKHKVYACEVTHQGLSSPVTKSFNRGES | |
| | HC(A) | QVQLVESGGGVVQPGRSLRLDCKASGITFSNSGMHW | 15 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVRPCAPSSKSTSGGTAALGCLVKD | |
| | | YFPEPVTVSWNSGALTSGVHTFPAVLQSSGLYSLSS | |
| | | VVTVPSSSLGTQTYICNVNHKPSNTKVDKKVEPKSS | |
| | | DKTHTCPPCPAPPVAGPSVFLFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRFSGSGSGTDF | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |
| DIC005 | LC(A) | EIVLTQSPATLSLSPGERATLSCRASQSVSSYLAWY | 16 |
| | | QQKPGQAPRLLIYDASNRATGIPARFSGSGSGTDET | |
| | | LTISSLEPEDFAVYYCQQSSNWPRTFGQGTKVEIKR | |
| | | TVAAPSVEICPPSDEQLKSGTASVVCLLNNFYPREA | |
| | | KVQWKVDNALQSGNSQESVTEQDSKDSTYSLSSTLT | |
| | | LSKADYEKHKVYACEVTHQGLSSPVTKSFNRGES | |
| | HC(A) | QVQLVESGGGVVQPGRSLRLDCKASGITFSNSGMHW | 17 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVFPCAPSSKSTSGGTARLGCLVKD | |
| | | YFPEPVTVSWNSGALTSGVHTFPAVLQSSGLYSLSS | |
| | | VVTVPSSSLGTQTYICNVNHKPSNTKVDKKVEPKSS | |
| | | DKTHTCPPCPAPPVAGPSVELFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRFSGSGSGTDF | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |
| DIC006 | LC(A) | EIVLTQSPATLSLSPGERATLSCRASQSVSSYLAWY | 18 |
| | | QQKPGQAPRLLIYDASNRATGIPARFSGSGSGTDET | |
| | | LTISSLEPEDFAVYYCQQSSNWPRTFGQGTKVEIKR | |
| | | TVAAPSVFICPPSKEQLKSGTASVVCLLNNFYPREA | |
| | | KVQWKVDNALQSGNSQESVTEQDSKDSTYSLSSTLT | |
| | | LSKADYEKHKVYACEVTHQGLSSPVTKSFNRGES | |
| | HC(A) | QVQLVESGGGVVQPGRSLRLDCKASGITESNSGMHW | 19 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVFPCAPSSKSTSGGTAALGCLVKD | |
| | | YFPEPVTVSWNSGALTSGVHTFPAVLQSSGLYSLSS | |
| | | VVTVPSSSLGTQTYICNVNHKPSNTKVDKKVEPDSS | |
| | | DKTHTCPPCPAPPVAGPSVFLFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRESGSGSGTDE | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |
| DIC009 | LC(A) | EIVLTQSPATLSLSPGERATLSCRASQSVSSYLAWY | 20 |
| | | QQKPGQAPRLLIYDASNRATGIPARFSGSGSGTDET | |
| | | LTISSLEPEDFAVYYCQQSSNWPRTFGQGTKVEIKR | |
| | | TVAAPSVFIFPPSDEQLKSGTASVVCLLNNFYPREA | |
| | | KVQWKVDNALQSGNSCESVTEQDSKDSTYSLDSTLT | |
| | | LSKADYEKHKVYACEVTHQGLSSPVTKSFNRGES | |
| | HC(A) | QVQLVESGGGVVQPGRSLRLDCKASGITESNSGMHW | 21 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVFPLAPSSKSTSGGTAALGCLVKD | |
| | | YFPEPVTVSWNSGALTSGVHTFPACLQSSGLYSLKS | |
| | | VVTVPSSSLGTQTYICNVNHKPSNTKVDKKVEPKSS | |
| | | DKTHTCPPCPAPPVAGPSVFLFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRESGSGSGTDF | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |
| DIC010 | LC(A) | EIVLTQSPATLSLSPGERATLSCRASQSVSSYLAWY | 22 |
| | | QQKPGQAPRLLIYDASNRATGIPARFSGSGSGTDFT | |
| | | LTISSLEPEDFAVYYCQQSSNWPRTFGQGTKVEIKR | |
| | | TVAAPSVDIFPPSDEQLKSGTASVVCLLNNFYPREA | |
| | | KVQWKVDNALQSGNSCESVTEQDSKDSTYSLSSTLT | |
| | | LSKADYEKHKVYACEVTHQGLSSPVTKSFNRGES | |
| | HC(A) | QVQLVESGGGVVQPGRSLRLDCKASGITFSNSGMHW | 23 |
| | | VRQAPGKGLEWVAVIWYDGSKRYYADSVKGRFTISR | |
| | | DNSKNTLFLQMNSLRAEDTAVYYCATNDDYWGQGTL | |
| | | VTVSSASTKGPSVFPLAPSSKSTSGGTAKLGCLVKD | |
| | | YFPEPVTVSWNSGALTSGVHTFPACLQSSGLYSLSS | |
| | | VVTVPSSSLGTQTYICNVNHKPSNTKVDKKVEPKSS | |
| | | DKTHTCPPCPAPPVAGPSVFLFPPKPKDTLMISRTP | |
| | | EVTCVVVDVSQEDPEVKFNWYVDGVEVHNAKTKPRE | |
| | | EQFNSTYRVVSVLTVLHQDWLNGKEYKCKVSNKGLP | |
| | | SSIEKTISKAKGQPREPQVYTLPPSRDELTKNQVSL | |
| | | TCLVKGFYPSDIAVEWESNGQPENNYDTTPPVLDSD | |
| | | GSFFLYSDLTVDKSRWQQGNVFSCSVMHEALHNHYT | |
| | | QKSLSLSP | |
| | LC(B) | ETVLTQSPGTLSLSPGERATLSCRASQSLGSSYLAW | 4 |
| | | YQQKPGQAPRLLIYGASSRAPGIPDRESGSGSGTDE | |
| | | TLTISRLEPEDFAVYYCQQYADSPITFGQGTRLEIK | |
| | | RTVAAPSVFIFPPSDEQLKSGTASVVCLLNNFYPRE | |
| | | AKVQWKVDNALQSGNSQESVTEQDSKDSTYSLSSTL | |
| | | TLSKADYEKHKVYACEVTHQGLSSPVTKSFNRGEC | |
| | HC(B) | QVQLVQSGAEVKKPGSSVKVSCKASGYTFSSNVISW | 8 |
| | | VRQAPGQGLEWMGGVIPIVDIANYAQRFKGRVTITA | |
| | | DESTSTTYMELSSLRSEDTAVYYCASTLGLVLDAMD | |
| | | YWGQGTLVTVSSASTKGPSVFPLAPSSKSTSGGTAA | |
| | | LGCLVKDYFPEPVTVSWNSGALTSGVHTFPAVLQSS | |
| | | GLYSLSSVVTVPSSSLGTQTYICNVNHKPSNTKVDK | |
| | | KVEPKSCDKTHTCPPCPAPPVAGPSVFLFPPKPKDT | |
| | | LMISRTPEVTCVVVDVSQEDPEVKFNWYVDGVEVHN | |
| | | AKTKPREEQFNSTYRVVSVLTVLHQDWLNGKEYKCK | |
| | | VSNKGLPSSIEKTISKAKGQPREPQVYTLPPSRKEL | |
| | | TKNQVSLTCLVKGFYPSDIAVEWESNGQPENNYKTT | |
| | | PPVLKSDGSFFLYSKLTVDKSRWQQGNVFSCSVMHE | |
| | | ALHNHYTQKSLSLSP | |

### a) Expression

The DNA sequences encoding four chains of DIC003, DIC004, DIC005, DIC006, DIC009 and DIC010 were cloned into vectors (at a ratio of 1:1:1:1), and transfected into ExpiCHO cells for transient expression, and then analyzed after AC purification.

### b) SDS-PAGE and SEC-HPLC Analysis

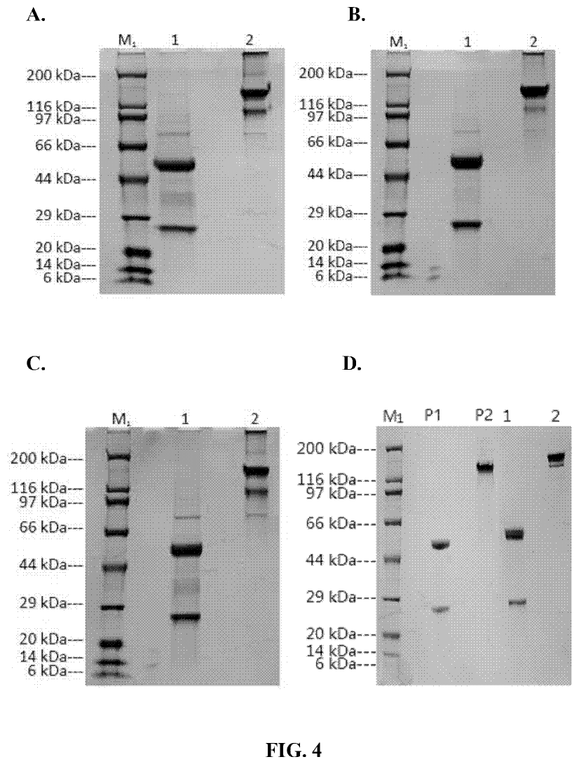

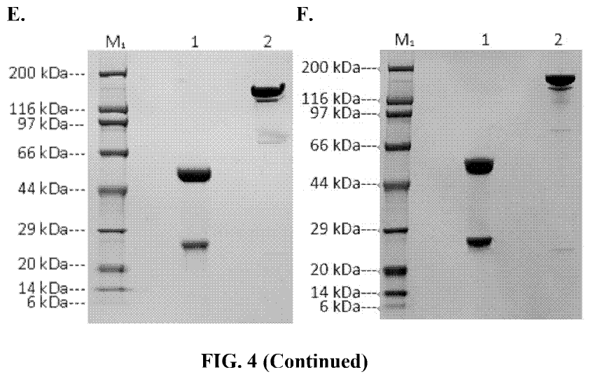

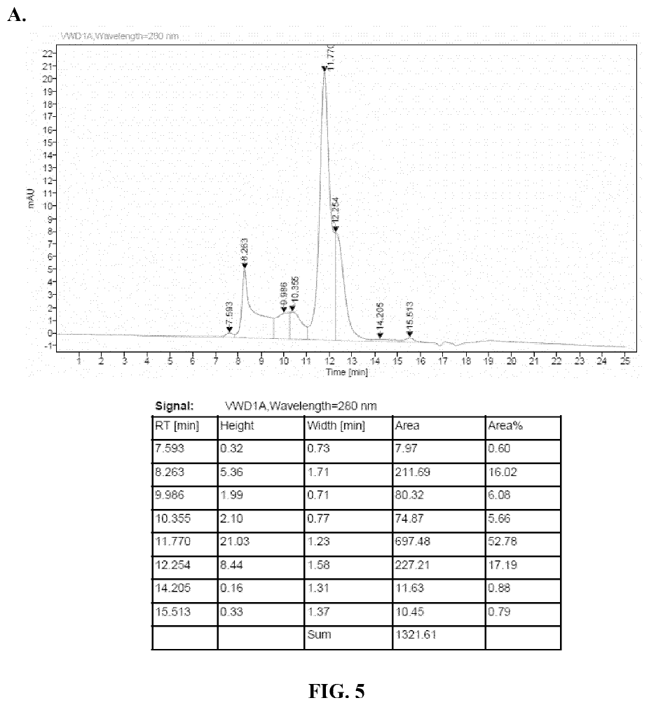

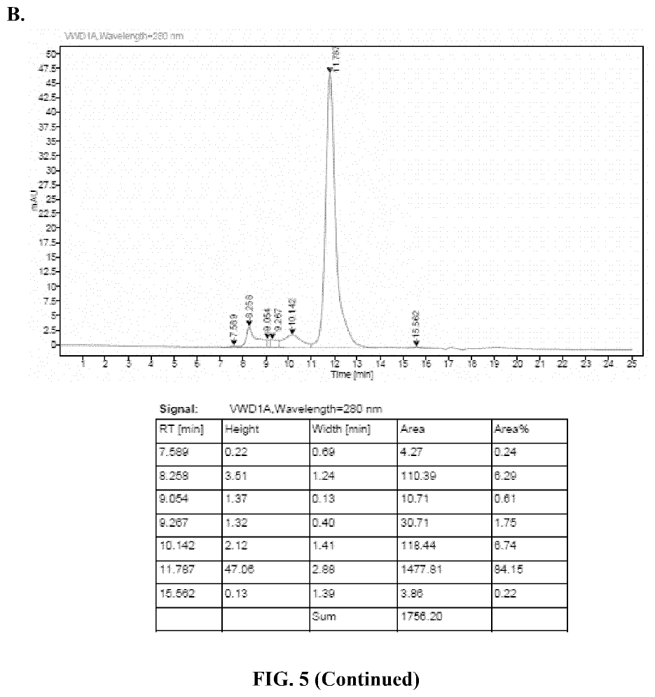

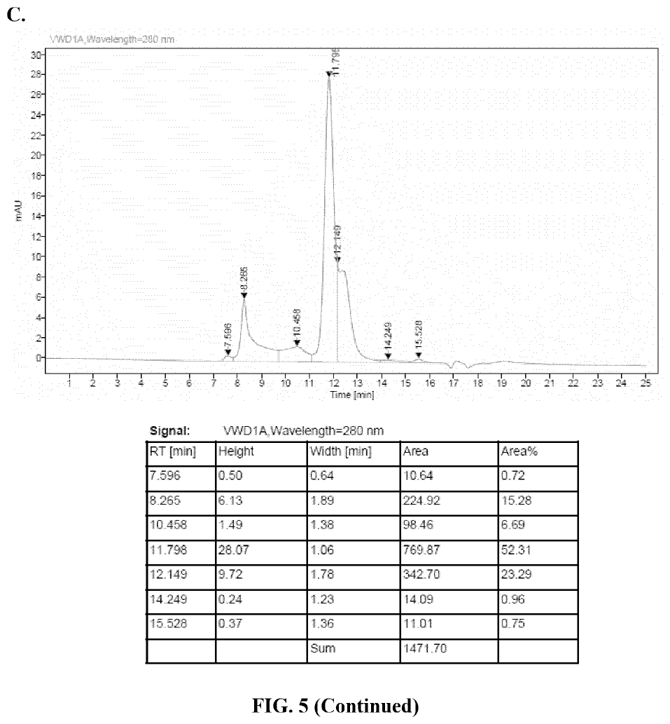

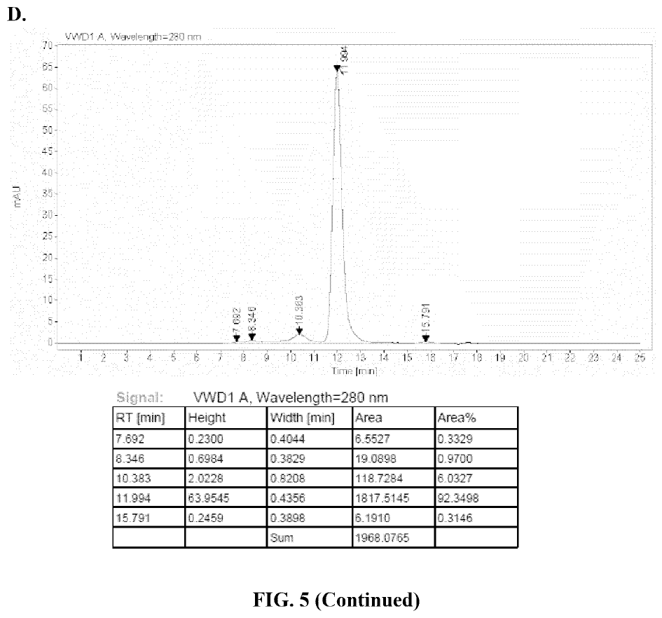

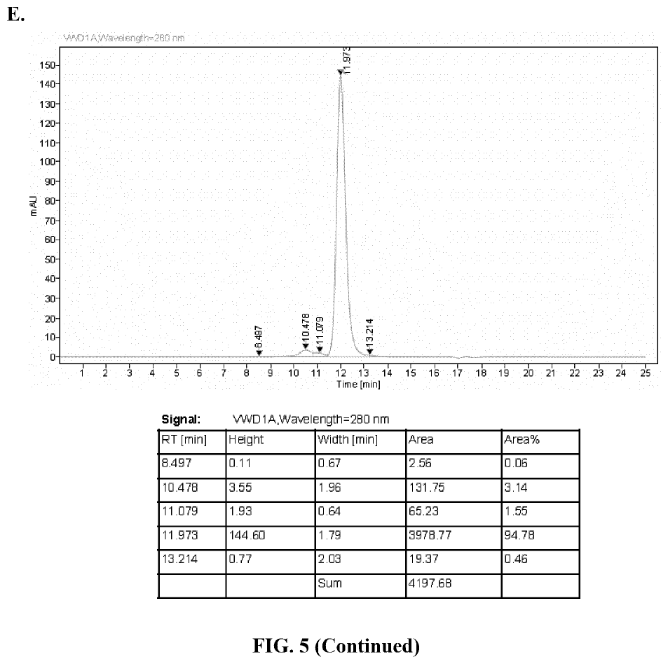

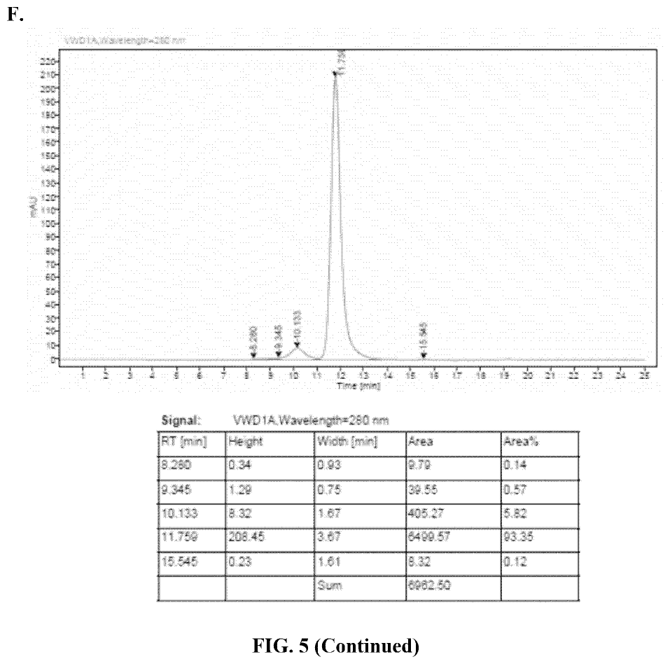

The purified antibodies above were detected by SDS-PAGE and SEC-HPLC, respectively. The SDS-PAGE results are shown in FIG. 4A (DIC003), FIG. 4B (DIC004), FIG. 4C (DIC005), FIG. 4D (DIC006), FIG. 4E (DIC009) and FIG. 4F (DIC010). The SEC-HPLC results are shown in FIG. 5A (DIC003), FIG. 5B (DIC004), FIG. 5C (DIC005), FIG. 5D (DIC006), FIG. 5E (DIC009) and FIG. 5F (DIC010).

The SDS-PAGE and SEC-HPLC analysis results of the antibodies above showed that, DIC006, DIC009 and DIC010 exhibited significant higher purities than DIC007 and DIC002. However, the methods above have obvious limitations for quantifying the contents and types of mispairings. For example, homodimers and CH1-CL mispairing cannot be distinguished from the target product by SDS-PAGE and SEC-HPLC due to their close molecular weights with the target product. Therefore, LC-MS method was used for discrimination and identification.

The left and right sides of DIC014 are connected by C220-C214, and thus the performance of SDS-PAGE and SEC-HPLC was similar to that of ordinary antibodies, and it is impossible to observe whether there is any mispairing. In order to identify the actual molecular weight of each mispairing product so as to study the composition of various mispairing products in the DIC009 product, based on the four chains of DIC009 (LC(A)-HC(A)-HC(B)-LC(B)), DIC015 expressing three chains of HC(A)-HC(B)-LC(B) and DIC016 expressing three chains of LC(A)-HC(A)-HC(B) were constructed as controls. As shown in Table 9 below, DIC015 did not have a LC(A) region, and DIC016 did not have a LC(B) region. For DIC015, V173C, C220S and S183D were introduced into the CH1(A) region; for DIC016, V173C, C220S and S183D were introduced into the CH1(A) region, Q160C, C214S and S176K were introduced into the LC(A) region.

TABLE 9
Mutations in CH1 and CL regions of DIC015 and DIC016

| Antibody Name | CH1(A) | LC(A) | CH1(B) | LC(B) |
|---|---|---|---|---|
| DIC015 | V173C, C220S | \ | — | — |
| | S183D | \ | — | — |
| DIC016 | V173C, C220S | Q160C, C214S | — | \ |
| | S183D | S176K | — | \ |

The SDS-PAGE results of DIC015 and DIC016 were shown in FIG. 6A (DIC015) and FIG. 6B (DIC016), respectively. As shown in FIGS. 6A and 6B, both DIC015 and DIC016 had the bands of 150 KDa, 125 KDa and 100 KDa. Because the products of 150 KDa are the mispairing products of HC(A)-LC(B) and HC(B)-LC(A) of DIC015 and DIC016, respectively, their amounts were lower as shown in SDS-PAGE.

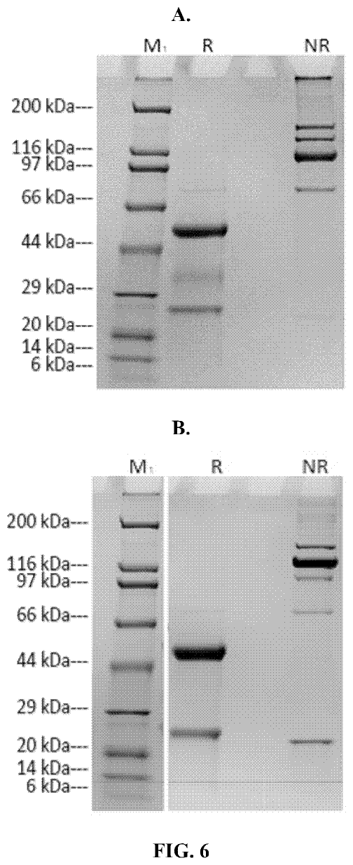

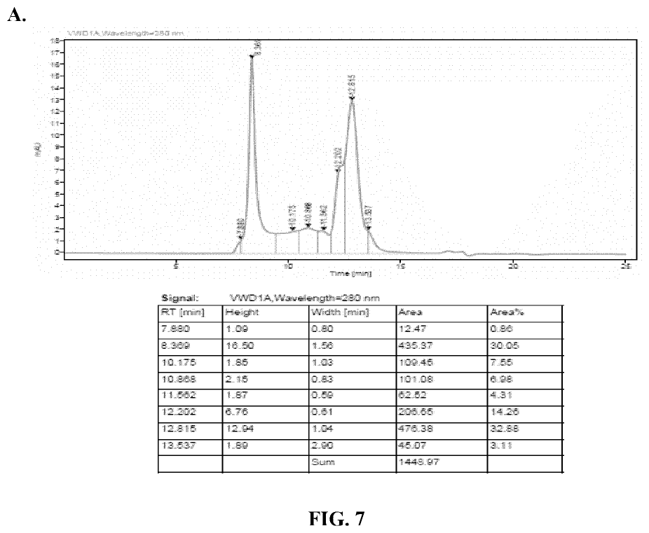

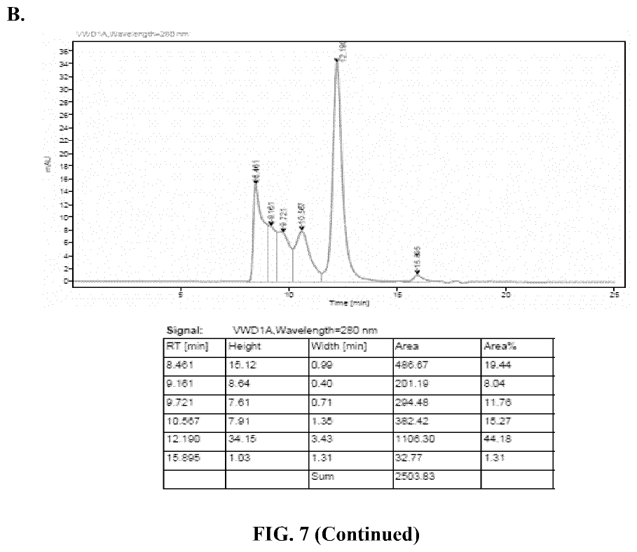

The SEC-HPLC results of DIC015 and DIC016 were shown in FIG. 7A (DIC015) and FIG. 7B (DIC016), respectively. As shown in FIGS. 7A and 7B, obvious dimers were observed for DIC015 and DIC016. DIC015 showed obvious shoulder peaks and main peaks around the target molecular weight, indicating that some of the HC(A)-LC(B) mispairing, although did not form a covalent bond, they exhibited a protein affinity binding form and showed a molecular weight of about 150 KDa (12.2 min); while part of HC(A) that does not bind to LC(B) formed a product of 125 KDa (12.8 min), with a small amount of 100 KDa product (13.5 min) of HC(A)-HC(B). DIC016 showed a single peak, indicating that a large amount of HC(B)-LC(A) mispairing formed a 150 KDa (12.2 min) mispairing product by protein affinity binding. The results of SDS-PAGE and SEC-HPLC methods showed that the methods above cannot effectively separate non-covalent mispairing binding products from the target final products. On this basis, the LC-MS analysis of DIC015 and DIC016 can be useful for determining the molecular weights of various mispairings and identifying the contents of mispairing products in DIC009.

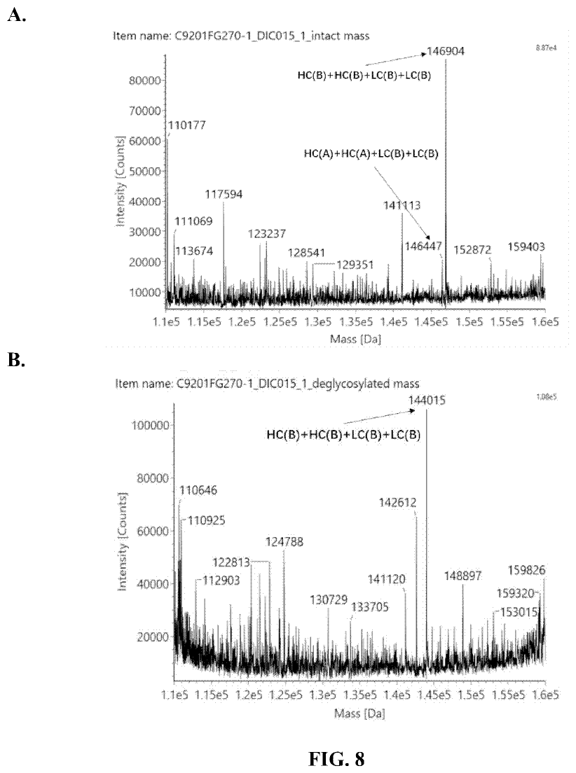

According to the compositions of DIC009, DIC015 and DIC016, the molecular weights of various possible mispairings were calculated to identify the composition of the final product. The molecular weight of each possible mispairing was shown in Table 10 below. The LC-MS results of intact DIC015 and deglycosylated DIC015 were shown in FIG. 8A (intact DIC015) and FIG. 8B (deglycosylated DIC015), respectively. The LC-MS results of intact DIC016 and deglycosylated DIC016 were shown in FIG. 9A (intact DIC015) and FIG. 9B (deglycosylated DIC016), respectively.

TABLE 10
The molecular weight of each possible product

| | Deglycosylated_expected |
|---|---|
| Complex Name | mass (Da) |
| HC(A) + HC(B) + LC(A) | 120181 |
| HC(A) + HC(B) + LC(B) | 120225 |
| HC(A) + HC(A) + LC(A) | 119795 |
| HC(A) + HC(A) + LC(B) | 119838 |
| HC(B) + HC(B) + LC(A) | 120568 |
| HC(B) + HC(B) + LC(B) | 120611 |
| HC(B) + HC(B) + LC(B) + LC(B) | 144010 |
| HC(B) + HC(B) + LC(A) + LC(B) | 143967 |
| HC(B) + HC(B) + LC(A) + LC(A) | 143924 |
| HC(A) + HC(B) + LC(A) + LC(A) | 143537 |
| HC(A) + HC(B) + LC(A) + LC(B) | 143580 |
| HC(A) + HC(B) + LC(B) + LC(B) | 143624 |
| HC(A) + HC(A) + LC(B) + LC(B) | 143237 |
| HC(A) + HC(A) + LC(A) + LC(B) | 143194 |
| HC(A) + HC(A) + LC(A) + LC(A) | 143151 |

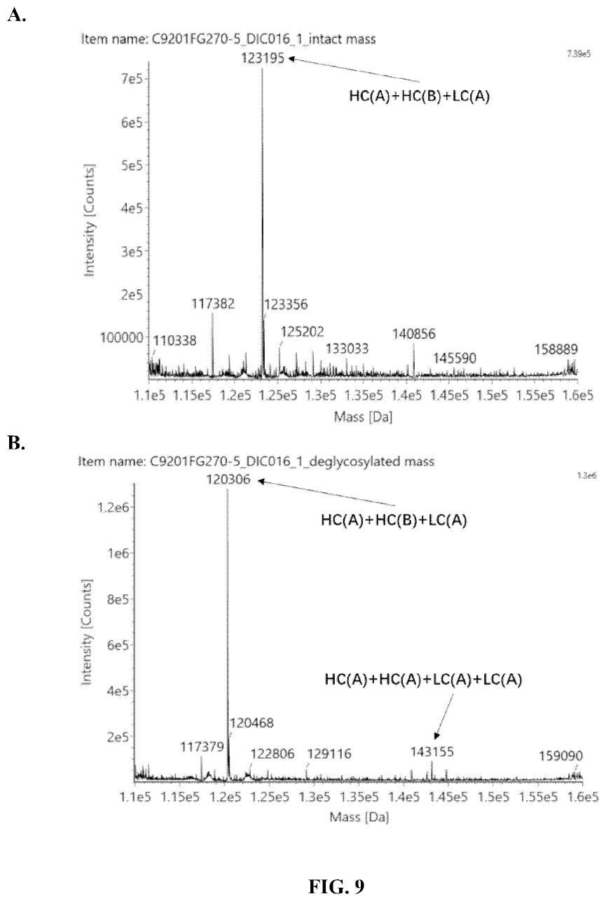

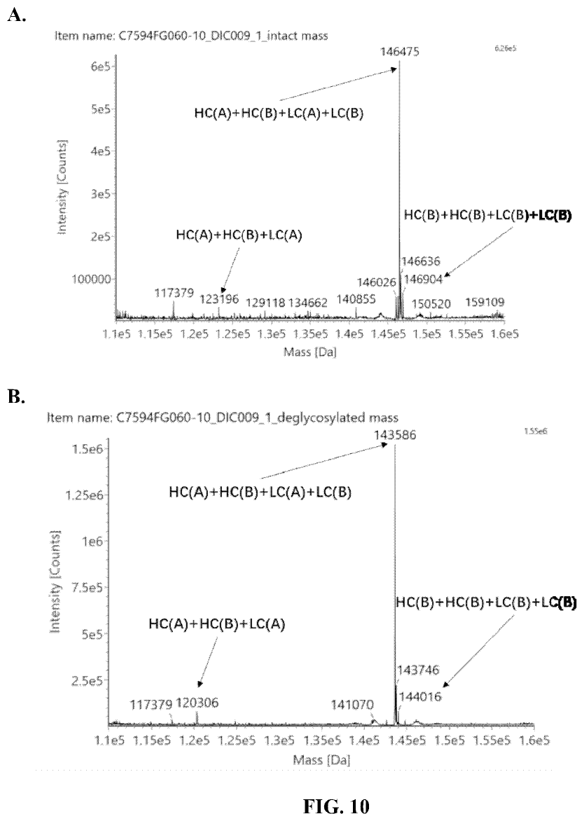

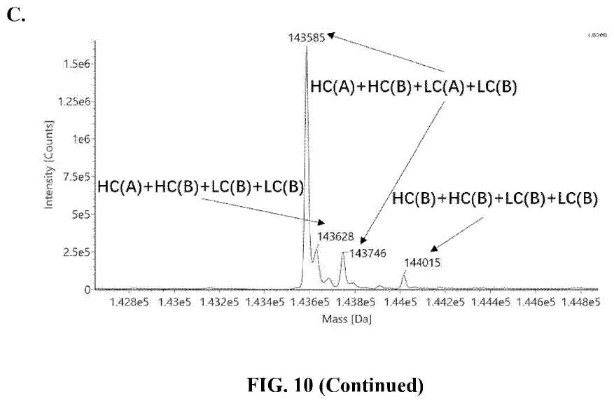

According to the data above, LC-MS analysis was conducted for DIC009 which expresses four chains so as to identify the mispairing ratio of the DIC009 product. The LC-MS results were shown in FIG. 10A (intact DIC009), FIG. 10B (deglycosylated DIC009), and FIG. 10C (partial enlarged FIG. 10B). As shown in FIG. 10, in addition to HC(A)-HC(B)-LC(A)-LC(B), the final product comprised some other by-products, including HC(A)-HC(B)-LC(B)-LC(B) and HC(B)-HC(B)-LC(B)-LC(B).

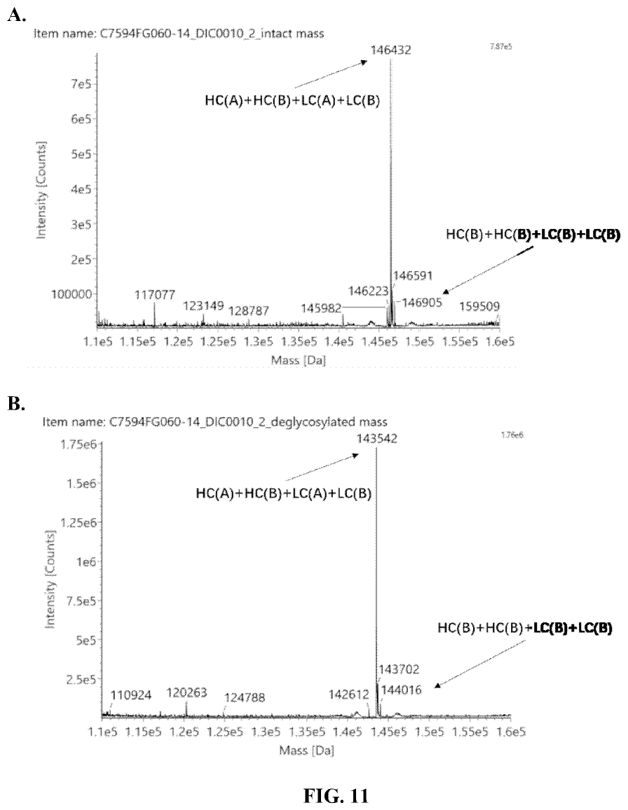

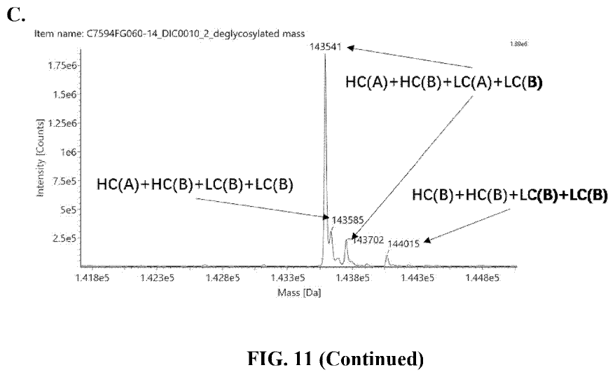

DIC010 was also detected by LC-MS, and the composition of the final product was analyzed by using similar methods above. The LC-MS results were shown in FIG. 11A (intact DIC010) and FIG. 11B (deglycosylated DIC010), and FIG. 11C (partial enlarged FIG. 11B). As shown in FIG. 11, the final product of DIC010 also comprised small amounts of mispairing products of HC(A)-HC(B)-LC(B)-LC(B) and HC(B)—HC(B)-LC(B)-LC(B).

Compared to DIC014 (which is without mutation), it can be found that the purities of DIC009 and DIC010 were significantly increased, and the amounts of mispairing products were decreased. Therefore, the construction methods of DIC009 and DIC010 can significantly decrease the mispairings of HC(A)-LC(B) and HC(B)-LC(A).

### Example 7: Biological Activity of DIC010

DIC010 constructed by the methods above was expressed and purified, and the biological activity of the constructed product was evaluated.

### a) Affinity

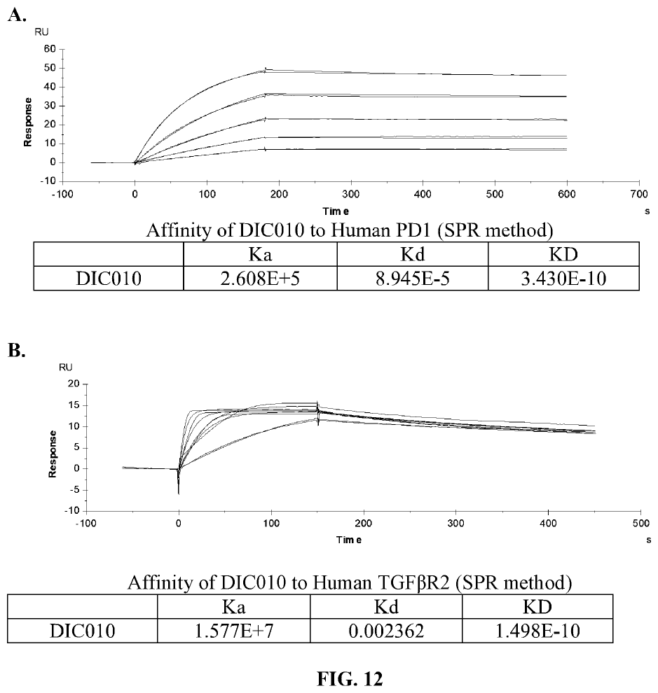

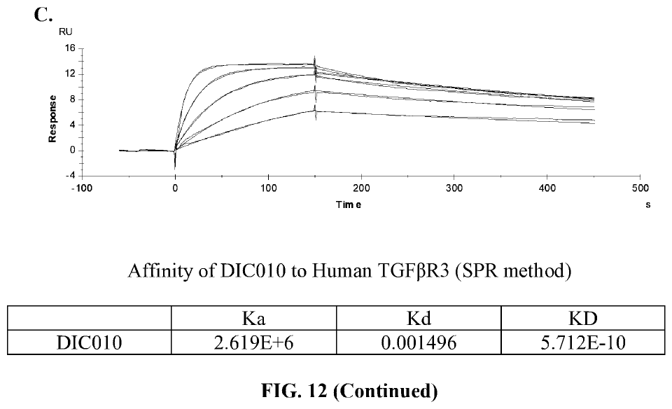

SPR method was used to detect binding affinities of DIC010 to human PD1, TGFβR2 and TGFβR3, and to observe whether the constructed bispecific product still retains the affinities of the original monoclonal antibodies to their respective targets. The SPR results were shown in FIG. 12A (human PD1), FIG. 12B (human TGFβR2), FIG. 12C (human TGFβR3). As shown in FIG. 12, DIC010 still showed good affinity to human PD1, human TGFβR2, and human TGFβR3.

### b) In Vivo Activity

1×10⁶ mouse colon cancer MC38/H-11 cells were injected into the left oxter of PD-1 single humanized mice. After the tumor grew to an average volume of 50-100 mm³, the animals were randomly grouped according to the tumor volumes. The mice were divided into 4 groups (6 mice/group): negative control group, DIC010 (1 mg/kg) group, DIC010 (3 mg/kg) group, and DIC010 (10 mg/kg) group. The animals in each group were intraperitoneally injected with the corresponding concentration of the test product at a dosage of 10 ml/kg, twice a week, for a total of 4 administrations, and the administration cycle was 15 days.

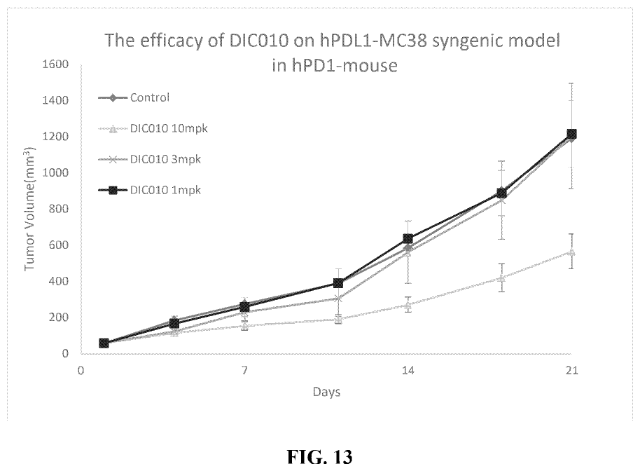

The mice were weighed and measured for tumor volumes twice a week. At Day 15, the mice were weighed, and the tumor volumes were measured to calculate relative tumor volume (RTV), relative tumor growth rate (T/C), and tumor growth inhibition rate (TGI). The results were shown in FIG. 13. As shown in FIG. 13, DIC010 10 mg/kg significantly inhibited tumor growth in mice MC38 model, and had good in vivo biological activity.

What claimed is:

1. A polypeptide complex comprising a first target-binding domain comprising a first target-binding moiety operably linked to a first constant moiety, wherein the first constant moiety comprises a first heavy chain constant region 1 (CH1) associated with a first light chain constant region (CL), wherein:
a) the first CH1 region comprises a first amino acid residue at EU position n1, and the first CL region comprises a second amino acid residue at EU position n2, wherein n1:n2 position pair is selected from the group consisting of 128:118 and 173:160, and wherein the first amino acid residue and the second amino acid residue form a covalent bond; and
b) the first CH1 region further comprises a third amino acid residue at EU position n3, and the first CL region further comprises a fourth amino acid residue at EU position n4, wherein n3:n4 position pair is selected from the group consisting of 183:176, 141:116, 126:121, and 218:122; wherein the third amino acid residue and the fourth amino acid residue form a non-covalent bond.

2. The polypeptide complex of claim 1, wherein the third amino acid residue at EU position n3 and the fourth amino acid residue at EU position n4 are oppositely charged.

3. The polypeptide complex of claim 1 or 2, further comprising a second target-binding domain comprising a second target-binding moiety operably linked to a second constant moiety, wherein the second constant moiety comprises a second CH1 region associated with a second CL region, wherein the first CH1 region does not substantially bind to the second CL region, and the second CH1 region does not substantially bind to the first CL region.

4. The polypeptide complex of claim 3, wherein the first or second target-binding domain comprises or is an antigen-binding domain, and/or, the first or second target-binding moiety comprises or is an antigen-binding moiety.

5. The polypeptide complex of claim 3 or 4, wherein the second CH1 region comprises a first corresponding amino acid residue at EU position n1′, and the second CL region comprises a second corresponding amino acid residue at EU position n2′, wherein n1′:n2′ position pair is identical to the n1:n2 position pair, and wherein the first corresponding amino acid residue at EU position n1′ does not form a covalent bond with the second amino acid residue at EU position n2, and/or the second corresponding amino acid residue at EU position n2′ does not form a covalent bond with the first amino acid residue at EU position n1.

6. The polypeptide complex of claim 5, wherein the first corresponding amino acid residue at EU position n1′ and the second corresponding amino acid residue at EU position n2′ do not form a covalent bond.

7. The polypeptide complex of claim 5 or 6, wherein the second CH1 region further comprises a third corresponding amino acid residue at EU position n3′, and the second CL region further comprises a fourth corresponding amino acid residue at EU position n4′, wherein the n3′:n4′ position pair is identical to the n3:n4 position pair, and wherein:
(a) the fourth corresponding amino acid residue at EU position n4′ and the third amino acid residue at EU position n3 are not oppositely charged or are like-charged, and/or
(b) the third corresponding amino acid residue at EU position n3′ and the fourth amino acid residue at EU position n4 are not oppositely charged or are like-charged.

8. The polypeptide complex of claim 7, wherein the third corresponding amino acid residue at EU position n3′ and/or the fourth corresponding amino acid residue at EU position n4′ are not charged.

9. The polypeptide complex of any of claims 3-8, wherein the second CH1 region further comprises a fifth corresponding amino acid residue at EU position n5′, and the second CL region further comprises a sixth corresponding amino acid residue at EU position n6′, and wherein the fifth corresponding amino acid residue and the sixth corresponding amino acid residue form a covalent bond, wherein n5′:n6′ position pair is different from the n1:n2 position pair.

10. The polypeptide complex of claim 9, wherein the n5′:n6′ position pair is selected from the group consisting of 220:214 (for IgG1) or 131:214 (for IgG2 and IgG4), 128:118 and 173:160.

11. The polypeptide complex of claim 10, wherein the n5′:n6′ position pair is 220:214 (for IgG1) or 131:214 (for IgG2 and IgG4).

12. The polypeptide complex of claim 10, wherein the n5′:n6′ position pair is 128:118, and the n1:n2 position pair is 173:160; or the n5′:n6′ position pair is 173:160, and the n1:n2 position pair is 128:118.

13. The polypeptide complex of claim 12, wherein:
(a) at least one of the first CH1 region and the second CH1 region has an amino acid residue other than cysteine at EU position 220 (for IgG1) or 131 (for IgG2 and IgG4), and/or at least one of the first CL region and the second CL region has an amino acid residue other than cysteine at EU position 214; or
(b) neither the first CH1 region nor the second CH1 region has a cysteine residue at EU position 220 (for IgG1) or 131 (for IgG2 and IgG4), and/or neither the first CL region nor the second CL region has a cysteine residue at EU position 214.

14. The polypeptide complex of claim 13, wherein the first CH1 region has an amino acid residue other than cysteine at EU position 220 (for IgG1) or 131 (for IgG2 and IgG4) and the first CL region has an amino acid residue other than cysteine at EU position 214.

15. The polypeptide complex of claim 13, wherein the second CH1 region has an amino acid residue other than cysteine at EU position 220 (for IgG1) or 131 (for IgG2 and IgG4) and the second CL region has an amino acid residue other than cysteine at EU position 214.

16. The polypeptide complex of any of claims 9-15, wherein the first CH1 region further comprises a fifth amino acid residue at EU position n5, and the first CL region further comprises a sixth amino acid residue at EU position n6, and wherein n5:n6 position pair is identical to the n5′:n6′ position pair, and wherein the fifth corresponding amino acid residue at EU position n5′ does not form a covalent bond with the sixth amino acid residue at EU position n6, and/or the sixth corresponding amino acid residue at EU position n6′ does not form a covalent bond with the fifth amino acid residue at EU position n5.

17. The polypeptide complex of claim 16, wherein the fifth amino acid residue at EU position n5 and the sixth amino acid residue at EU position n6 do not form a covalent bond.

18. The polypeptide complex of any one of claims 3-17, wherein the second CH1 region further comprises a seventh corresponding amino acid residue at EU position n7′ and the second CL region further comprises an eighth corresponding amino acid residue at EU position n8′, wherein n7′:n8′ position pair is selected from the group consisting of 183:176, 141:116, 126:121, and 218:122; wherein the seventh corresponding amino acid residue and the eighth corresponding amino acid residue are oppositely charged, and wherein the n7′:n8′ position pair is different from the n3:n4 position pair.

19. The polypeptide complex of claim 18, wherein:
(a) the n7′:n8′ position pair is 183:176, and the n3:n4 position pair is selected from the group consisting of 141:116, 126:121, and 218:122;
(b) the n7′:n8′ position pair is 141:116, and the n3:n4 position pair is selected from the group consisting of 183:176, 126:121, and 218:122;
(c) the n7′:n8′ position pair is 126:121, and the n3:n4 position pair is selected from the group consisting of 183:176, 141:116, and 218:122; or
(d) the n7′:n8′ position pair is 218:122, and the n3:n4 position pair is selected from the group consisting of 183:176, 141:116, and 126:121.

20. The polypeptide complex of claim 18, wherein the first CH1 region further comprises a seventh amino acid residue at EU position n7, and the second CL region further comprises an eighth amino acid residue at EU position n8, wherein the n7:n8 position pair is identical to the n7′:n8′ position pair, and wherein the seventh corresponding amino acid residue at EU position n7′ and the eighth amino acid residue at EU position n8 are not oppositely charged or are like-charged, and/or the eighth corresponding amino acid residue at EU position n8′ and the seventh amino acid residue at EU position n7 are not oppositely charged or are like-charged.

21. The polypeptide complex of claim 20, wherein the seventh amino acid residue at EU position n7 and/or the eighth amino acid residue at EU position n8 are not charged.

22. The polypeptide complex of any of the preceding claims, wherein covalent bond is a disulfide bond.

23. The polypeptide complex of claim 22, wherein the disulfide bond is formed between two cysteine residues.

24. The polypeptide complex of claim 23, wherein the first amino acid residue at EU position n1 and the second amino acid residue at EU position n2 are both cysteine residues, and/or the fifth corresponding amino acid residue at EU position n5′ and the sixth corresponding amino acid residue at EU position n6′ are both cysteine residues.

25. The polypeptide complex of claim 24, wherein the first CH1 region comprises a substitution of L128C (EU position n1) and the first CL region comprises a substation of F118C (EU position n2).

26. The polypeptide complex of claim 24, wherein the second CH1 region comprises a substitution of V173C (EU position n5′) and the second CL region comprises a substation of Q160C (EU position n6′) for a kappa light chain or E160C (EU position n6′) for a lambda light chain.

27. The polypeptide complex of any of preceding claims, wherein:
(a) the third amino acid residue at EU position n3 is a positive-charged amino acid residue, and the fourth amino acid residue at EU position n4 is a negative-charged amino acid residue; or
(b) the third amino acid residue at EU position n3 is a negative-charged amino acid residue, and the fourth amino acid residue at EU position n4 is a positive-charged amino acid residue.

28. The polypeptide complex of any of claims 18-27, wherein:
(c) the seventh corresponding amino acid residue at EU position n7′ is a positive-charged amino acid residue, and the eighth corresponding amino acid residue at EU position n8′ is a negative-charged amino acid residue; or
(d) the seventh corresponding amino acid residue at EU position n7′ is a negative-charged amino acid residue, and the eighth corresponding amino acid residue at EU position n8′ is a positive-charged amino acid residue.

29. The polypeptide complex of claim 27 or 28, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

30. The polypeptide complex of any one of the preceding claims, wherein at least one, two, three, or four of the first amino acid residue at EU position n1, the second amino acid residue at EU position n2, the third amino acid residue at EU position n3, and fourth amino acid residue at EU position n4 are introduced by substitution.

31. The polypeptide complex of claim 30, wherein the third amino acid residue and the fourth amino acid residue at the n3:n4 position pair are substitutions selected from the group consisting of: S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R.

32. The polypeptide complex of any one of the preceding claims 16-31, wherein at least one, two, three, or four of the fifth corresponding amino acid residue at EU position n5′, the sixth corresponding amino acid residue at EU position n6′, the seventh corresponding amino acid residue at EU position n7′, and the eighth corresponding amino acid residue at EU position n8′ are introduced by substitution.

33. The polypeptide complex of claim 32, wherein the seventh corresponding amino acid residue and the eighth corresponding amino acid residue at the n7′:n8′ position pair are substitutions selected from the group consisting of: S183K:S176D, S183K:S176E, S183R:S176D, S183R:S176E, S183H:S176D, S183H:S176E, S183D:S176K, S183D:S176R, S183D:S176H, S183E:S176K, S183E:S176R, S183E:S176H, A141K:F116D, A141K:F116E, A141R:F116D, A141R:F116E, A141H:F116D, A141H:F116E, A141D:F116K, A141D:F116R, A141D:F116H, A141E:F116K, A141E:F116R, A141E:F116H, F126K:S121D, F126K:S121E, F126R:S121D, F126R:S121E, F126H:S121D, F126H:S121E, F126D:S121K, F126D:S121R, F126D:S121H, F126E:S121K, F126E:S121R, F126E:S121H, K218D:D122K, K218D:D122H, K218D:D122R, K218E:D122K, K218E:D122H, and K218E:D122R, and wherein the n7′:n8′ position pair is different from the n3:n4 position pair.

34. The polypeptide complex of any of the preceding claims, wherein the first target-binding domain comprises a first combination of substitutions at (n1+n2):(n3+n4) positions, and/or the second target-binding domain comprises a second combination of substitutions at (n5′+n6′):(n7′+n8′) positions, and wherein the first combination of substitutions and/or the second combination of substitutions are selected from the group consisting of: (L128C+S183K):(F118C+S176D), (L128C+S183K):(F118C+S176E), (L128C+S183R):(F118C+S176D), (L128C+S183R):(F118C+S176E), (L128C+S183H):(F118C+S176D), (L128C+S183H):(F118C+S176E), (L128C+S183D):(F118C+S176K), (L128C+S183D):(F118C+S176R), (L128C+S183D):(F118C+S176H), (L128C+S183E):(F118C+S176K), (L128C+S183E):(F118C+S176R), (L128C+S183E):(F118C+S176H), (V173C+A141K):(Q160C (or E160C)+F116D), (V173C+A141K):(Q160C (or E160C)+F116E), (V173C+A141R):(Q160C (or E160C)+F116D), (V173C+A141R):(Q160C (or E160C)+F116E), (V173C+A141H):(Q160C (or E160C)+F116D), (V173C+A141H):(Q160C (or E160C)+F116E), (V173C+A141D):(Q160C (or E160C)+F116K), (V173C+A141D):(Q160C (or E160C)+F116R), (V173C+A141D):(Q160C (or E160C)+F116H), (V173C+A141E):(Q160C (or E160C)+F116K), (V173C+A141E):(Q160C (or E160C)+F116R), (V173C+A141E):(Q160C (or E160C)+F116H), (V173C+S183K):(Q160C (or E160C)+S176D), (V173C+S183K):(Q160C (or E160C)+S176E), (V173C+S183R):(Q160C (or E160C)+S176D), (V173C+S183R):(Q160C (or E160C)+S176E), (V173C+S183H):(Q160C (or E160C)+S176D), (V173C+S183H):(Q160C (or E160C)+S176E), (V173C+S183D):(Q160C (or E160C)+S176K), (V173C+S183D):(Q160C (or E160C)+S176R), (V173C+S183D):(Q160C (or E160C)+S176H), (V173C+S183E):(Q160C (or E160C)+S176K), (V173C+S183E):(Q160C (or E160C)+S176R), (V173C+S183E):(Q160C (or E160C)+S176H), (L128C+F126K):(F118C+S121D), (L128C+F126K):(F118C+S121E), (L128C+F126R):(F118C+S121D), (L128C+F126R):(F118C+S121E), (L128C+F126H):(F118C+S121D), (L128C+F126H):(F118C+S121E), (L128C+F126D):(F118C+S121K), (L128C+F126D):(F118C+S121R), (L128C+F126D):(F118C+S121H), (L128C+F126E):(F118C+S121K), (L128C+F126E):(F118C+S121R), (L128C+F126E):(F118C+S121H), (V173C+F126K):(Q160C (or E160C)+S121D), (V173C+F126K):(Q160C (or E160C)+S121E), (V173C+F126R):(Q160C (or E160C)+S121D), (V173C+F126R):(Q160C (or E160C)+S121E), (V173C+F126H):(Q160C (or E160C)+S121D), (V173C+F126H):(Q160C (or E160C)+S121E), (V173C+F126D):(Q160C (or E160C)+S121K), (V173C+F126D):(Q160C (or E160C)+S121R), (V173C+F126D):(Q160C (or E160C)+S121H), (V173C+F126E):(Q160C (or E160C)+S121K), (V173C+F126E):(Q160C (or E160C)+S121R), (V173C+F126E):(Q160C (or E160C)+S121H), (L128C+K218D):(F118C+D122K), (L128C+K218D):(F118C+D122H), (L128C+K218D):(F118C+D122R), (L128C+K218E):(F118C+D122K), (L128C+K218E):(F118C+D122H), (L128C+K218E):(F118C+D122R), (V173C+K218D):(Q160C (or E160C)+D122K), (V173C+K218D):(Q160C (or E160C)+D122H), (V173C+K218D):(Q160C (or E160C)+D122R), (V173C+K218E):(Q160C (or E160C)+D122K), (V173C+K218E):(Q160C (or E160C)+D122H), and (V173C+K218E):(Q160C (or E160C)+D122R),
provided that, when both the first combination of substitutions and the second combination of substitutions are selected, the n5′:n6′ position pair is different from the n1:n2 position pair, and the n7′:n8′ position pair is different from the n3:n4 position pair.

35. The polypeptide complex of any of the preceding claims, wherein the first target-binding moiety comprises a first polypeptide fragment operably linked to the first CL region, and/or the second target-binding moiety comprises a second polypeptide fragment operably linked to the second CL region, wherein the first polypeptide fragment has a different amino acid sequence from the second polypeptide fragment, or either the first polypeptide fragment or the second polypeptide fragment is absent from the polypeptide complex.

36. The polypeptide complex of claim 35, wherein the first target-binding moiety further comprises a third polypeptide fragment operably linked to the first CH1 region, and/or the second target-binding moiety comprises a fourth polypeptide fragment operably linked to the second CH1 region.

37. The polypeptide complex of claim 36, wherein the third polypeptide fragment has a different amino acid sequence from the fourth polypeptide fragment; or either the third polypeptide fragment or the fourth polypeptide fragment is absent from the polypeptide complex.

38. The polypeptide complex of any of claims 35-37, wherein the first polypeptide fragment and the third polypeptide fragment each contains a first target-binding site or associate with each other to form a first target-binding site; and/or the second polypeptide fragment and the fourth polypeptide fragment each contains a second target-binding site or associate with each other to form a second target-binding site.

39. The polypeptide complex of any of claims 35-37, wherein the first target-binding site and the second target-binding site can bind to the same target molecule, or different parts on the same target molecule, or different target molecules.

40. The polypeptide complex of any of claims 4-39, wherein the first antigen-binding domain and/or the second antigen-binding domain is contained within an antibody, optionally a bispecific antibody or a multispecific antibody.

41. The polypeptide complex of any of claims 4-40, wherein the second antigen-binding domain and the first antigen-binding domain bind to different antigens or bind to different epitopes on the same antigen.

42. The polypeptide complex of claim 41, wherein the antigen can be a tumor-associated antigen, immune related target, or an infectious agent related target.

43. The polypeptide complex of any one of claims 4-42, wherein the first and/or the second antigen-binding domain is chimeric, humanized, or fully human.

44. The polypeptide complex of any one of claims 4-43, wherein the first and/or the second antigen-binding moiety is selected from the group consisting of a nanobody, an Fv fragment, a scFv, a disulfide stabilized Fv fragment, a (dsFv)₂, a bispecific dsFv, and a diabody.

45. The polypeptide complex of any one of claims 4-44, wherein the first and/or the second antigen-binding domain is selected from the group consisting of a Fab domain, a Fab′, and a F(ab′)₂.

46. The polypeptide complex of claim 45, wherein the first antigen-binding domain and/or the second antigen-binding domain comprises one or more CDRs operably linked to a CH1 region and a CL region.

47. The polypeptide complex of any of claims 4-46, wherein the first antigen-binding domain is a first Fab domain, and/or the second antigen-binding domain is a second Fab domain.

48. The polypeptide complex of claim 47, wherein the second Fab domain comprises:
(a) one or more light chain CDRs and/or light chain framework regions different from that of the first Fab domain; and optionally,
(b) one or more heavy chain CDRs and/or heavy chain framework regions different from that of the first Fab domain.

49. The polypeptide complex of any of the preceding claims, further comprising an Fc region operably linked to the first target-binding domain and the second target-binding domain.

50. The polypeptide complex of claim 49, wherein the Fc region is derived from IgG1, IgG2, IgG3 or IgG4.

51. The polypeptide complex of claim 49 or 50, wherein the Fe region is heterodimeric.

52. The polypeptide complex of claim 49, wherein the heterodimeric Fc region comprises one or more mutations that facilitate heterodimerization.

53. The polypeptide complex of claim 52, wherein the heterodimeric Fc region comprises a first Fc polypeptide comprising a first Fc mutation, and/or a second Fe polypeptide comprising a second Fc mutation, wherein:
a) the first Fe mutations comprises T366W or S354C, and the second Fe mutation comprises Y349C, T366S, L368A, or Y407V;
b) the first Fc mutation comprises D399K or E356K, and the second Fc mutation comprises K392D, or K409D;
c) the first Fe mutation comprises E356K, E357K, or D399K, and the second Fe mutation comprises K370E, K409D, or K439E;
d) the first Fc mutation comprises S364H, or F405A, and the second Fc mutation comprises Y349T, or T394F;
e) the first Fc mutation comprises S364H, or T394F, and the second Fe mutation comprises Y394T, or F405A;
f) the first Fc mutation comprises K370D, or K409D, and the second Fc mutation comprises E357K, or D399K; or
g) the first Fc mutation comprises L351D, or L368E, and the second Fc mutation comprises L351K, or T366K,
wherein numbering is according to the EU index.

54. A nucleic acid comprising a nucleotide sequence encoding the polypeptide complex of any one of claims 1-53 or a part thereof.

55. A vector comprising the nucleic acid of claim 54.

56. A host cell comprising the nucleic acid of claim 54 or the vector of claim 55.

57. A pharmaceutical composition comprising the polypeptide complex of any one of claims 1-53 and a pharmaceutically acceptable carrier.

58. A conjugate comprising the polypeptide complex of any one of claims 1-53 and a payload conjugated thereto, wherein the payload is selected from the group consisting of a radioactive label, a fluorescent label, an enzyme-substrate label, an affinity purification tag, a tracer molecule, an anticancer drug, and a cytotoxic molecule.

59. A composition comprising the polypeptide complex of any one of claims 1-53, or the conjugate of claim 58, and a pharmaceutically acceptable carrier.

60. A method of treating or preventing from a disease, condition, or symptom comprising administering to a subject in need thereof a therapeutically effective amount of the polypeptide complex of any one of claims 1-53, the pharmaceutical composition of claim 51, the conjugate of claim 58, or the composition of claim 59.

61. The method of claim 60, wherein the disease is selected from the group consisting of a cancer, an inflammatory disease, an infectious or parasitic disease, a cardiovascular disease, neuropathy, a neuropsychiatric condition, an injury, an autoimmune disease, a metabolic disease, a neurodegenerative disease, or a coagulation disorder.

62. A method of detecting presence or level of an antigen, comprising contacting a sample suspected of containing the antigen with the polypeptide complex of any one of claims 1-53, and determining the formation of a complex between the antigen and the polypeptide complex.
